(19) United States
(12) Patent Application Publication
(10) Pub. No.: US 2020/0194117 A1
(43) Pub. Date: Jun. 18, 2020

(54) SYSTEMS, METHODS, AND MEDIA FOR REMOTE TRAUMA ASSESSMENT

(71) Applicants: University of Maryland, College Park, College Park, MD (US); University of Maryland, Baltimore, Baltimore, MD (US)

(72) Inventors: Axel Krieger, Alexandria, VA (US); Thorsten Roger Fleiter, Owings Mills, MD (US); Hamed Saeidi, College Park, MD (US); Bharat Mathur, Hyattsville, MD (US); Saul Schaffer, New Orleans, LA (US); Anirudh Topiwala, College Park, MD (US); Michael Kam, Greenbelt, MD (US); Lidia Al-Zogbi, College Park, MD (US)

(21) Appl. No.: 16/714,134

(22) Filed: Dec. 13, 2019

Related U.S. Application Data
(60) Provisional application No. 62/779,306, filed on Dec. 13, 2018.

(51) Int. Cl. G16H 40/67; G16H 30/20; G06N 3/08; G16H 50/20

(52) U.S. Cl. CPC G16H 40/67; G16H 50/20; G06N 3/08; G16H 30/20

(57) ABSTRACT

In some embodiments, systems and methods for remote trauma assessment are provided, a system comprising, a robot arm; an ultrasound probe and a depth sensor coupled to the robot arm; and a processor programmed to: cause the depth sensor to acquire depth data indicative of a three dimensional shape of at least a portion of a patient; generate a 3D model of the patient; automatically identify scan positions using the 3D model; cause the robot arm to move the ultrasound probe to a first identified scan position; receive movement information indicative of input provided via a remotely operated haptic device; cause the robot arm to move the ultrasound probe from the first scan position to a second position based on the movement information; cause the ultrasound probe to acquire ultrasound data at the second position; and transmit an ultrasound image based on the ultrasound data to the remote computing device.

### CROSS-REFERENCE TO RELATED APPLICATIONS

This application is based on, claims the benefit of, and claims priority to U.S. Provisional Application No. 62/779,306, filed Dec. 13, 2018, which is hereby incorporated by reference herein in its entirety for all purposes.

### STATEMENT REGARDING FEDERALLY SPONSORED RESEARCH

N/A

### BACKGROUND

Unintentional injury and trauma continues to be a widespread issue throughout the United States. For example, estimates for the year 2016 indicate that unintentional injury or trauma was the third leading cause of death in the United States. This estimate does not favor a particular subset, and rather spans across all age groups (1-44 years old) and populations. Of the unintentional injuries and traumas, unintentional motor vehicle and traffic accidents, unintentional falls and injuries, and firearm injuries were the groups with the highest likelihood for morbidity and mortality.

Typically, the probability for survival rapidly diminishes based on the severity of the trauma incident, and the time elapsed since the trauma incident. For example, highly severe trauma incidents (e.g., hemorrhage), require more prompt attention when compared to less severe incidents, as the patient's condition can rapidly deteriorate. As another example, according to the Golden Hour concept (e.g., as described by R Adams Cowley of the University of Maryland) the longer the elapsed time since a trauma incident, the lower the probability of survival. Thus, it is imperative to minimize the time between the start of the traumatic incident and the initiation of appropriate medical care.

Accordingly improved systems, methods, and media for remote trauma assessment are desirable.

### SUMMARY

In accordance with some embodiments of the disclosed subject matter, systems, methods, and media for remote trauma assessment are provided.

In accordance with some embodiments of the disclosed subject matter, a system for remote trauma assessment is provided, the system comprising: a robot arm; an ultrasound probe coupled to the robot arm a depth sensor; a wireless communication system; and a processor that is programmed to: cause the depth sensor to acquire depth data indicative of a three dimensional shape of at least a portion of a patient; generate a 3D model of the patient based on the depth data; automatically identify, without user input, a plurality of scan positions using the 3D model; cause the robot arm to move the ultrasound probe to a first scan position of the plurality of scan positions; receive, from a remote computing device via the wireless communication system, movement information indicative of input to the remote computing device provided via a remotely operated haptic device; cause the robot arm to move the ultrasound probe from the first scan position to a second position based on the movement information; cause the ultrasound probe to acquire ultrasound signals at the second position; and transmit ultrasound data based on the acquired ultrasound signals to the remote computing device.

In some embodiments, the system further comprises a force sensor coupled to the robot arm, the force sensor configured to sense a force applied to the ultrasound probe and in communication with the processor.

In some embodiments, the processor is further programmed to: inhibit remote control of the robot arm while the force applied to the ultrasound probe is below a threshold; determine that the force applied to the ultrasound probe at the first position exceeds the threshold based on a force value received from the force sensor; and in response to determining that the force applied to the ultrasound probe at the first position exceeds the threshold, accept movement information from the remote computing device.

In some embodiments, the system further comprises a depth camera comprising the depth sensor, and the 3D model is a 3D point cloud, and wherein the processor is further programmed to: cause the robot arm to move the depth camera to a plurality of positions around a patient; cause the depth camera to acquire the depth data and corresponding image data at each of the plurality of positions; generate the 3D point cloud based on the depth data and the image data; determine a location of the patient's umbilicus using image data depicting the patient; determine at least one dimension of the patient using the 3D point cloud; identify the plurality of scan positions based on the location of the patient's umbilicus, the at least one dimension, and a labeled atlas.

In some embodiments, the processor is further programmed to: provide an image of the patient to a trained machine learning model, wherein the trained machine learning model is a detection network (e.g., a Faster R-CNN) that was trained to identify a region of an image corresponding to an umbilicus using labeled training images depicting umbilici; receive, from the trained machine learning model, an output indicating a location of the patient's umbilicus within the image; and map the location of the patient's umbilicus within the image to a location on the 3D model.

In some embodiments, the processor is further programmed to: provide an image of the patient to a trained machine learning model, wherein the trained machine learning model is a Faster R-CNN that was trained to identify a region of an image corresponding to a wound using labeled training images depicting wounds; receive, from the trained machine learning model, an output indicating a location of a wound within the image; map the location of the wound within the image to a location on the 3D model; and cause the robot arm to avoid moving the ultrasound probe within a threshold distance of the wound.

In some embodiments, the processor is further programmed to: generate an artificial potential field emerging from the location of the wound and having a field strength that decreases with distance from the wound; determine, based on a position of the ultrasound probe, a force exerted on the ultrasound probe by the artificial potential field; transmit force information indicative of the force exerted by on the ultrasound probe by the artificial potential field to the remote computing device thereby causing the force exerted on the ultrasound probe by the artificial potential field to be provided as haptic feedback by the haptic device.

In some embodiments, the processor is further programmed to: provide an image of the patient to a trained machine learning model, wherein the trained machine learning model was trained to identify regions of an image corresponding to objects to be avoided during an ultrasound procedure using labeled training images depicting objects to be avoided during an ultrasound procedure; receive, from the trained machine learning model, an output indicating one or more locations corresponding to objects to avoid within the image; map the one or more locations within the image to a location on the 3D model; and cause the robot arm to avoid moving the ultrasound probe within a threshold distance of the one or more locations.

In some embodiments, the processor is further programmed to: receive a force value from the force sensor indicative of the force applied to the ultrasound probe; and transmit force information indicative of the force value to the remote computing device such that the remote computing device displays information indicative of force being applied by the ultrasound probe.

In some embodiments, the system further comprises a camera, and the processor is further programmed to: receive an image of the patient from the camera; format the image of the patient for input to an automated skin segmentation model to generate a formatted image; provide the formatted image to the automated skin segmentation model, wherein the automated skin segmentation model is a segmentation network (e.g., a U-Net-based model) trained using a manually segmented dataset of images that includes a plurality of images that each depict an exposed human abdominal region; receive, from the automated skin segmentation model, a mask indicating which portions of the image correspond to skin; and label at least a portion of the 3D model as corresponding to skin based on the mask.

In accordance with some embodiments of the disclosed subject matter, a method for remote trauma assessment is provided, the method comprising: causing a depth sensor to acquire depth data indicative of a three dimensional shape of at least a portion of a patient; generating a 3D model of the patient based on the depth data; automatically identifying, without user input, a plurality of scan positions using the 3D model; causing the robot arm to move an ultrasound probe mechanically coupled to a distal end of the robot arm to a first scan position of the plurality of scan positions; receiving, from a remote computing device via a wireless communication system, movement information indicative of input to the remote computing device provided via a remotely operated haptic device; causing the robot arm to move the ultrasound probe from the first scan position to a second position based on the movement information; causing the ultrasound probe to acquire ultrasound signals at the second position; and transmitting ultrasound data based on the acquired ultrasound signals to the remote computing device.

In accordance with some embodiments of the disclosed subject matter, a system for remote trauma assessment is provided, the system comprising: a haptic device having at least five degrees of freedom; a user interface; a display; and a processor that is programmed to: cause a graphical user interface comprising a plurality of user interface elements to be presented by the display, the plurality of user interface elements including a switch; receive, from a remote mobile platform over a communication network, an instruction to enable actuation of the switch; receive, via the user interface, input indicative of actuation of the switch; receive, from the haptic device, input indicative of at least one of a position and orientation of the haptic device; and in response to receiving the input indicative of actuation of the switch, transmit movement information based on the input indicative of at least one of the position and orientation of the haptic device to the mobile platform.

In some embodiments, the haptic device includes an actuatable switch, the actuatable switch having a first position, and a second position, and the processor is further programmed to: in response to the actuatable switch being in the first position, cause a robot arm associated with the mobile platform to inhibit translational movements of an ultrasound probe coupled to a distal end of the robot arm along a first axis, a second axis, and a third axis; and in response to the actuatable switch being in the second position, cause the robot arm to accept translational movement commands that cause the ultrasound probe to translate along at least one of the first axis, the second axis, and the third axis.

In some embodiments, the plurality of user interface elements includes a selectable first user interface element corresponding to a first location, and wherein the processor is further programmed to: receive, via the user interface, input indicative of selection of the first user interface element; and in response to receiving the input indicative of selection of the first user interface element, cause a robot arm associated with the mobile platform to autonomously move an ultrasound probe coupled to a distal end of the robot arm to a first position associated with the first user interface element.

In some embodiments, the processor is further programmed to: receive force information indicative of a force value generate by a force sensor associated with the mobile device and the robot arm, wherein the force value is indicative of a normal force acting on the ultrasound probe; and cause the display to present information indicative of the force value based on the information indicative of the force value.

In some embodiments, the processor is further programmed to: determine that the force value does not exceed a threshold, and in response to determining that the force value does not exceed a threshold, cause the display to present information indicating that the ultrasound probe is not in contact with a patient to be scanned.

In some embodiments, the processor is further programmed to: receive, from the mobile platform, image data acquired by a camera associated with the mobile platform, wherein the image data depicts at least a portion of the ultrasound probe; receive, from the mobile platform, ultrasound data acquired by the ultrasound probe; and cause the display to simultaneously present an image based on the image data and an ultrasound image based on the ultrasound image.

### BRIEF DESCRIPTIONS OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

The patent or application file contains at least one drawing executed in color. Copies of this patent or patent application publication with color drawing(s) will be provided by the Office upon request and payment of the necessary fee.

FIG. 1 shows an example of a trauma assessment system in accordance with some embodiments of the disclosed subject matter.

FIG. 2 shows an example of hardware that can be used to implement a computing device and a mobile platform shown in FIG. 1 in accordance with some embodiments of the disclosed subject matter.

FIG. 3 shows a schematic illustration of a remote trauma assessment flow in accordance with some embodiments of the disclosed subject matter.

FIG. 4A shows an example of another schematic illustration of a remote trauma assessment flow in accordance with some embodiments of the disclosed subject matter.

FIG. 4B shows an example of yet another schematic illustration of a remote trauma assessment flow in accordance with some embodiments of the disclosed subject matter.

FIG. 5 shows an example of a flowchart of a process for remote trauma assessment using a robotic system in accordance with some embodiments of the disclosed subject matter.

FIG. 6 shows an example of a flowchart of a process for automatically determining FAST scan locations for a robotic system in accordance with some embodiments of the disclosed subject matter.

FIG. 7 shows an example of FAST scan locations plotted on a 3D point cloud using an atlas, in accordance with some embodiments of the disclosed subject matter.

FIG. 8 shows an example of a flowchart of a process for training and utilizing a machine learning model in accordance with some embodiments of the disclosed subject matter.

FIG. 9 shows an example of a flow for training and utilizing a machine learning model in accordance with some embodiments of the disclosed subject matter.

FIG. 10 shows examples of images that have been used to train a machine learning model to classify images and extract candidate regions in accordance with some embodiments of the disclosed subject matter, and images classified using a machine learning model trained in accordance with some embodiments of the disclosed subject matter.

FIG. 11 shows an example of a process for tele-manipulation of a robotic system in accordance with some embodiments of the disclosed subject matter.

FIG. 12A shows an example of a portion of a graphical user interface presented by a display in accordance with some embodiments of the disclosed subject matter.

FIG. 12B shows an example of another portion of a graphical user interface presented by a display in accordance with some embodiments of the disclosed subject matter.

FIG. 13A shows an example of a remote portion of a trauma assessment system implemented in accordance with some embodiments of the disclosed subject matter.

FIG. 13B shows an example of a remote portion of a trauma assessment system, as shown in FIG. 13A, with labeled components and joints, in accordance with some embodiments of the disclosed subject matter.

FIG. 14A shows an example of a haptic device with a labeled coordinate system, in accordance with some embodiments of the disclosed subject matter.

FIG. 14B shows an example of the haptic device of FIG. 14A, with joints J4, J5, J6 and corresponding rotational terms, in accordance with some embodiments of the disclosed subject matter.

FIG. 15 shows examples of coordinate systems for a haptic device, for a mobile camera, and for a robot arm in accordance with some embodiments of the disclosed subject matter.

FIG. 16A shows a graph of mean average precision vs. intersection over union (“IoU”) for images of bandages, umbilici, and wounds in accordance with some embodiments of the disclosed subject matter.

FIG. 16B shows a graph of precision vs. recall with IoU held constant at 0.5 in accordance with some embodiments of the disclosed subject matter.

FIG. 17 shows a graph of forces recorded during a FAST scan at position three, during an example in accordance with some embodiments of the disclosed subject matter.

FIG. 18 shows comparisons of labeled ultrasound images, where ultrasound images on the left were acquired using mechanisms for remote trauma assessment implemented in accordance with some embodiments of the disclosed subject matter, and images on the right were acquired from a hand scan.

FIG. 19 shows a series of graded ultrasound scans with images in the top row corresponding to ultrasound scans acquired with a remote trauma assessment system implemented in accordance with some embodiments of the disclosed subject matter and ultrasound images in the bottom row corresponding to ultrasound scans acquired manually by an expert radiologist.

FIG. 20 shows an example of a positioning test procedure used to assess a remote trauma assessment system implemented in accordance with some embodiments of the disclosed subject matter.

FIG. 21 shows an example of a graphical user interface (“GUI”) implemented in accordance with some embodiments of the disclosed subject matter.

FIG. 22 shows an example of a graph of pitch angles with respect to time for one test while using a virtual fixture in accordance with some embodiments of the disclosed subject matter.

FIG. 23 shows examples of heat maps of forces and trajectories during tele-manipulation of a robotic system implemented in accordance with some embodiments of the disclosed subject matter using artificial potential fields.

FIG. 24 shows an example of a process for automatically labeling portions of an image corresponding to human skin in accordance with some embodiment of the disclosed subject matter.

FIG. 25A shows an example of a robot arm with an ultrasound probe and a camera positioned to acquire images of a simulated human torso in accordance with some embodiments of the disclosed subject matter.

FIG. 25B shows an example of an image of the simulated human torso depicted in FIG. 25A.

FIG. 25C shows a mask corresponding to the image of FIG. 25B generated using techniques described herein for automatically labeling portions of an image corresponding to human skin in accordance with some embodiments of the disclosed subject matter.

FIG. 26 shows examples of images depicting human abdomens that can be used to train an automated skin segmentation model in accordance with some embodiments of the disclosed subject matter.

FIG. 27A shows an example of an RGB histogram for pixels labeled as being non-skin in a dataset of human abdominal images that can be used to train an automated skin segmentation model in accordance with some embodiments of the disclosed subject matter.

FIG. 27B shows an example of an RGB histogram for pixels labeled as being skin in a dataset of human abdominal images that can be used to train an automated skin segmentation model in accordance with some embodiments of the disclosed subject matter.

FIG. 28 shows a table of a distribution of images from various datasets used to train and evaluate various automated skin segmentation models implemented in accordance with some embodiments of the disclosed subject matter.

FIG. 29 shows a boxplot depicting the accuracy achieved by various different automated skin segmentation models trained and evaluated using images from the datasets described in connection with FIG. 28, in accordance with some embodiments of the disclosed subject matter.

FIG. 30 shows a table of evaluation results for various different automated skin segmentation models trained and evaluated using images from the different combinations of datasets described in connection with FIG. 28, and comparisons with other reported skin segmentation accuracies, in accordance with some embodiments of the disclosed subject matter.

FIG. 31A shows an example of Receiver Operating Characteristic (“ROC”) curves for automated skin segmentation models trained with and without images from the abdominal dataset described in connection with FIG. 28, in accordance with some embodiments of the disclosed subject matter.

FIG. 31B shows an example of Precision-Recall curves for automated skin segmentation models trained with and without images from the abdominal dataset described in connection with FIG. 28, in accordance with some embodiments of the disclosed subject matter.

FIG. 32 shows an example of accuracy achieved during training of various versions of an automated skin segmentation model in a 10-fold cross validations experiment across 200 iterations of training, in accordance with some embodiments of the disclosed subject matter.

FIG. 33 shows an example of a loss value calculated during training of various versions of an automated skin segmentation model in a 10-fold cross validations experiment across 200 iterations of training, in accordance with some embodiments of the disclosed subject matter.

FIG. 34 shows examples of images of human abdomens and masks generated using various techniques for automatically labeling portions of an image corresponding to human skin in accordance with some embodiments of the disclosed subject matter.

FIG. 35 shows examples of images of human abdomens, manually labeled ground truth masks, and automatically segmented masks generated using an automated skin segmentation model trained in accordance with some embodiments of the disclosed subject matter.

FIG. 36 shows examples of images of human abdomens, manually labeled ground truth masks, and automatically segmented masks generated using an automated skin segmentation model trained in accordance with some embodiments of the disclosed subject matter with and without images from the abdominal dataset described in connection with FIG. 28.

FIG. 37 shows examples of frames of video and corresponding automatically segmented masks generated in real-time using an automated skin segmentation model trained in accordance with some embodiments of the disclosed subject matter.

### DETAILED DESCRIPTION

As described above, the likelihood of surviving a severe trauma incident typically decreases as the time elapsed increases (e.g., from the start of the incident to when medical care is administered). Thus, timing, although not entirely controllable in many situations, is critical. The implementation of statewide emergency medical services has vastly improved patient outcomes by attempting to optimize initial care and providing fast transport to dedicated trauma centers. Ultimately, emergency medical services significantly reduce prehospital time for trauma victims, which thereby decreases the total elapsed time from the start of the incident to when medical care is administered. However, although emergency medical services have improved patient outcomes, emergency medical services cannot mitigate all problems. For example, even the fastest and best organized emergency medical services cannot transport a patient to a trauma center fast enough to prevent all patient deaths. Of all pre-hospital deaths, 29% are estimated to be classified as potentially survivable, being attributed to uncontrolled hemorrhages that are not readily unidentified and/or treated by emergency medical technicians.

Additionally, in some cases, upon arriving at the hospital, the trauma patient must undergo diagnostic imaging procedures to understand the extent of the trauma patient's injuries, and to determine a diagnosis for an appropriate treatment. At times, the trauma patient must wait for various steps during the diagnostic process. For example, a trauma patient may have to wait for an imaging system to become available. As another example, a trauma patient may have to wait during the imaging procedure. As yet another example, a trauma patient may have to wait following the imaging procedure while the results is being analyzed (e.g., to determine a diagnosis).

Various diagnostic imaging procedures have been proposed to effectively diagnose trauma patients. For example focused assessment with ultrasound for trauma (sometimes referred to as “FAST” or “eFAST”) is a relatively well established, and accepted diagnostic procedure. The FAST procedure is desirable for both its simplicity and speed. Additionally, the FAST procedure can be used to identify collapsed lungs (pneumothorax), and can identify significant hemorrhage in the chest, abdomen, pelvis, and pericardium.

In general, the FAST technique focuses on imaging four major scan locations on a patient, which can include (1) an area of the abdomen below the umbilicus, (2) an area of the chest above the umbilicus, (3) an area on a right side of the back ribcage, and (4) an area on a left side of the back ribcage. Although, compact and mobile ultrasound systems could be used at the initial point of care, first responders tasked with utilizing the ultrasound systems would require extensive teaching to use such systems. Accordingly, even though first responders could theoretically implement the FAST technique, the first responders lack the experience required to produce ultrasound images that can be used for diagnostic purposes, and regardless of the image quality, to accurately interpret the resulting image. Aside from imaging difficulties and diagnostic difficulties, requiring first responders to implement a FAST technique redirects attention away from tasks typically implemented by first responders that are known to improve outcomes for patients that would not benefit from a FAST scan (e.g., patients without hidden hemorrhages). For example, first responders typically stabilize the patient, provide compression for hemorrhage control, and initiate cardiopulmonary resuscitation. Considering that typical tasks completed by first responders can be far more important than a theoretical FAST scan, in many cases forcing first responders to implement FAST scans would not be advantageous, and could potentially cause harm.

Delays in generating appropriate diagnostic information for first responders, and other medical practitioners, can often be problematic, when the diagnosis is not visually apparent. For example, without sufficient diagnostic information, first responders cannot initiate the necessary initial treatment for the underlying diagnosis, and thus cannot effectively address the injuries causing a potential hemorrhage, or loss of blood volume. Additionally as described above the trauma patient must undergo diagnostic imaging, potentially requiring wait times before and/or after arriving at the hospital, before obtaining a diagnosis. This can be especially problematic for specific trauma injuries, such as occult thoracic or abdominal injuries (e.g., caused by high energy mechanisms, such as automotive crashes), penetrating injuries (e.g., gun shot and stab wounds), and blunt trauma injuries, all of which can result in significant hemorrhage. Without a proper diagnosis for these injuries, potentially life-saving invasive techniques such as placement of a resuscitative endovascular balloon occlusion of the aorta (“REBOA”), or self-expanding foam for the treatment of severe intra-abdominal hemorrhage, may be delayed or cannot be administered in time (e.g., the trauma patient's condition has deteriorated beyond a particular level).

In some embodiments, systems, methods, and media for remote trauma assessment can be provided. For example, in some embodiments, a robotic imaging system can perform FAST scanning at the initial point of care (e.g., within an emergency medical service vehicle), with remote control of the robotic imaging system by a trained user (e.g., an ultrasound technician, a radiologist, etc.). In a more particular example, the robotic imaging system can be controlled remotely by an experienced practitioner (e.g., an ultrasound technician, a radiologist, etc.). In such an example, the experienced practitioner can manipulate a haptic device (e.g., another robot) to a desired orientation. Movements and/or positions of the haptic device can be relayed to the robotic imaging system, and the robotic imaging system can initiates the relayed movements. In some embodiments, the tactile proficiency of an experienced practitioner can facilitate utilization of mechanisms described herein for remote trauma assessment at the initial point of care, and thus the robotic imaging system can be used to produce diagnostically sufficient images. Additionally, a diagnosis can be made (e.g., by a radiologist) well before the trauma patient arrives at the hospital. This can allow first responders the ability to initiate appropriate care while transporting the patient to the hospital, and allowing the hospital sufficient time to prepare for potentially life-saving procedures while the trauma patient is traveling (e.g., REBOA, self-expanding foam, etc.), by providing diagnostic information while the patient is still in transit.

In some embodiments, a robotic imaging system can have a force sensor, which can be used to provide force information to a remote user. For example, generally, if too much or too little force is applied while depressing an ultrasound probe on a patient, the quality of the ultrasound images can be negatively affected. Thus, in some embodiments, a computing device can present the force data to the user as feedback and/or limit the amount of force that can be applied to address negative effects that excessive force can have on the quality of the acquired ultrasound images. In some embodiments, the robotic imaging system can have a camera, which can image the trauma patient. Images from such a camera can be analyzed to determine locations to avoid while acquiring ultrasound images of the patient. For example, the robotic imaging system, or other suitable computing device, can extract and label regions within the images acquired by the camera that correspond to regions that are not suitable for ultrasound imaging. In some embodiments, the robotic imaging system can use labelling information to avoid contacting these regions (and/or or contacting the region with a force value above a threshold) during ultrasound imaging. For example, such regions can correspond to wounds, bandages, etc., and contacting such regions may exacerbate an underlying problem (e.g., may cause further bleeding).

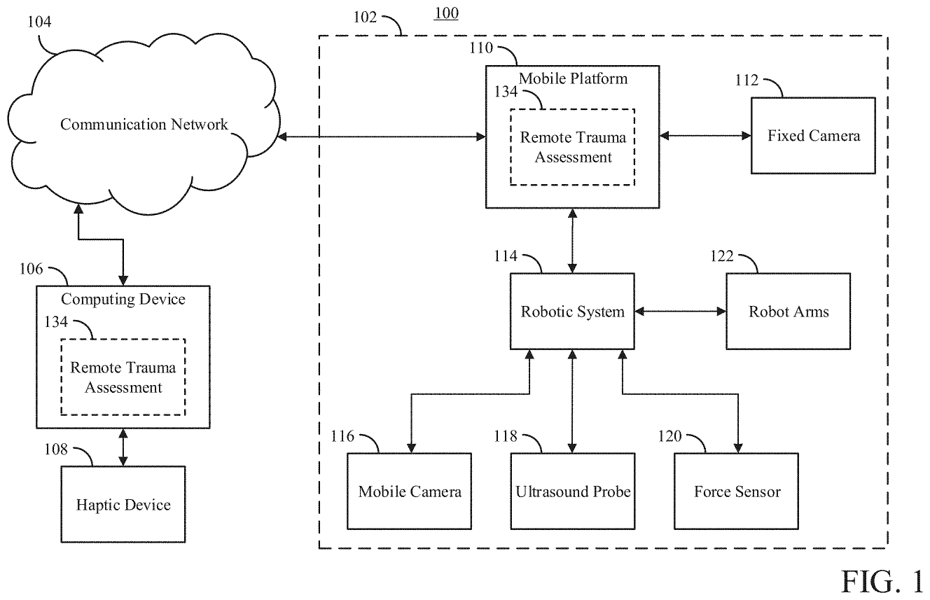

FIG. 1 shows an example 100 of a trauma assessment system in accordance with some embodiments of the disclosed subject matter. In some embodiments, the trauma assessment system 100 can include a robotic imaging system 102, a communication network 104, a computing device 106, and a haptic device 108. The robotic imaging system 102 can include a mobile platform 110, a fixed camera 112, a robotic system 114, a mobile camera 116, an ultrasound probe 118, a force sensor 120, and one or more robot arms 122. As shown in FIG. 1, the mobile platform 110 can be in communication (e.g., wired communication, wireless communication) with components of the robotic imaging system 102. In a more particular example, the mobile platform 110 can implement portions of a remote trauma assessment application 134, which can involve the mobile platform 110 transmitting and/or receiving instructions, data, commands, sensor values, etc., from one or more other devices (e.g., such as the computing device 106). For example, mobile platform 110 can cause the cameras 112, 116 to acquire images, cause the ultrasound probe 118 to acquire one or more ultrasound images, receive force sensor values from the force sensor 120, cause the robot arm 122 to move, and sensing a position of the robot arm 122. Although not shown in FIG. 1, the mobile platform 110 and the other components of the robotic imaging system 102 may reside within an emergency vehicle (e.g., an ambulance), and thus the mobile platform 110 may communicate with one or more components of the emergency vehicle as appropriate.

In some embodiments, the fixed camera 112 can be mounted to a structure above the imaging scene (e.g., where a trauma patient is positioned) of the robotic imaging system 102 and away from the robotic system 114 (e.g., above the robotic system 114). For example, the fixed camera 112 can be mounted to an interior surface of an emergency vehicle. As another example, the camera 112 can be mounted to a fixed structure, such that the fixed structure can allow the camera 112 to be positioned above the robotic imaging system 102. In such examples, the robotic system 114 does not interfere with the acquisition of an image with the fixed camera 112 (e.g., by entirely blocking a field of view of fixed camera 112). In some embodiments, the fixed camera 112 can be implemented using any suitable camera technology to acquire two-dimensional image data. For example, the fixed camera 112 can be a two-dimensional (“2D”) color camera. As another example, the fixed camera 112 can acquire images using various wavelengths of light (e.g., infrared light, visible light, etc.). In a more specific example, the fixed camera 112 can be implemented using a Chameleon CMLN-13S2C color camera (available from Sony Corporation, Tokyo, Japan). Additionally or alternatively, in some embodiments, the fixed camera 112 can be implemented using any suitable camera technology to acquire three-dimensional image data using a stereoscopic camera, a monocular camera, etc., and can detect one or more wavelengths of light (e.g., infrared light, visible light, etc.). For example, the fixed camera 112 can be implemented using a stereoscopic camera that includes stereoscopically positioned image sensors, to acquire 3D imaging data (e.g., by using triangulation on corresponding images acquired from the stereoscopically positioned image sensors). As another example, the fixed camera 112 can be implemented using a depth camera that can acquire 3D imaging data (e.g., using continuous time-of-flight imaging depth sensing techniques, using structured light depth sensing techniques, using discrete time of flight depth sensing techniques, etc.). In a more particular example, the fixed camera 112 can implemented using a RealSense D415 RGB-D camera (available from Intel Corporation, Santa Clara, Calif.).

As shown in FIG. 1, the mobile camera 116, the ultrasound probe 118, and the force sensor 120 can be mechanically and/or electrically coupled to the robotic system 114. The robotic system 114 can generally include a single robot arm 122, although in some configurations, the robotic system 114 can include multiple robot arms. In some embodiments, the robot arm 122 can include any suitable number of robotic segments (e.g., six, seven, etc.), where each robotic segment can control at least one degree of freedom of the given robot arm 122. For example, the robotic system 114 can include a single robot arm 122 implemented with seven segments, and seven corresponding degrees of freedom. In a more example, the robot arm 122 can be implemented using a seven degree of freedom (“DOF”) medical light weight robot (“LWR”) (e.g., a 7 DOF robot arm available from KUKA AG, Augsburg Germany). In some embodiments, the robotic system 114 interfaces with the mobile platform 110, such that the mobile platform 110 can receive information from, and send commands to, the robotic system 114, the mobile camera 116, the ultrasound probe 118, the force sensor 120, and/or the robot arm 122. In some embodiments the robot arm 122 can be implemented as a parallel manipulator (e.g., a delta robot) in place of a serial manipulator.

In some embodiments, the mobile camera 116 can be coupled (and/or mounted) to the robot arm 122. For example, the mobile camera 116 can be mounted to a specific segment of the robot arm 122 that also can include and/or implement the end effector (“EE”) of the robotic system 114 (e.g., the end effector can be mounted to the same segment). In some embodiments, mobile camera 116 can be any suitable camera that can be used to acquire three-dimensional (“3D”) imaging data of the trauma patient and corresponding visual (e.g., color) image data of the trauma patient, using any suitable technique or combinations of techniques. For example, the mobile camera 116 can be implemented using a stereoscopic camera, a monocular camera, etc., and can detect one or more wavelengths of light (e.g., infrared light, visible light, etc.). In a more particular example, the mobile camera 116 can be implemented using a stereoscopic camera that includes stereoscopically positioned image sensors, to acquire 3D imaging data (e.g., by using triangulation on corresponding images acquired from the stereoscopically positioned image sensors). As another example, the mobile camera 116 can be implemented using a depth camera that can acquire 3D imaging data (e.g., using continuous time-of-flight imaging depth sensing techniques, using structured light depth sensing techniques, using discrete time of flight depth sensing techniques, etc.). In a more particular example, the mobile camera 116 can implemented using a RealSense D415 RGB-D camera (available from Intel Corporation, Santa Clara, Calif.). In some embodiments, in lieu of or in addition to depth information from the fixed camera 112 and/or the mobile camera 116, the mobile platform 110 can be associated with one or more depth sensors (not shown) that can be used to generate depth information indicative of a shape of a patient (e.g., a patient located in a particular location with respect to the robot arm 122). For example, such depth sensors can include one or more sonar sensors, one or more ultrasonic detectors, one or more LiDar-based detectors, etc. In such embodiments, depth information form depth sensors can be used in connection with images from the fixed camera 112 and/or the mobile camera 116 to generate a 3D model of a patient.

In some embodiments, the ultrasound probe 118 can be coupled (and/or mounted) to a particular segment of the robot arm 122. In some embodiments, the ultrasound probe 118 can be implemented as the end effector of the robotic system 114 (e.g., the ultrasound probe 118 can be mounted to the robotic segment most distal from the origin or base of the robot arm 122). In some embodiments, the ultrasound probe 118 can include a processor, piezoelectric transducers, etc., that cause the ultrasound probe to emit an ultrasound signal and/or receive an ultrasound signal (e.g., after interacting with the patient's anatomy), to generate ultrasound imaging data, etc. In a particular example, the ultrasound probe 118 can be implemented using a CMS600P2 Portable Ultrasound Scanner (available at Contec Medical Systems Co. Ltd, Hebei, China), having a 3.5 MHz convex probe for ultrasound imaging. In some embodiments, the ultrasound probe 118 can be mounted to the last joint of the robot (e.g., coaxial with the last joint), and the mobile camera 116 can be mounted to the last joint (or segment) of the robot arm 122. For example, the mobile camera 116 can be mounted proximal to (with respect to the base of the robot arm 122) the ultrasound probe 118 on the same segment of the robot arm 122, such that the mobile camera 116 can acquire images of the ultrasound probe 118, and a scene surrounding at least a portion of the ultrasound probe 118 (e.g., images of the ultrasound probe in context).

In some embodiments, the force sensor 120 can be coupled (and/or mounted) to a particular segment of the robot arm 122, within the robotic system 114. For example, the force sensor 120 can be positioned and mounted to the last joint (or robotic segment) of the robot arm 122, of the robotic system 114. In a more particular example, the force sensor 120 can be mounted to a proximal end of (and coaxially with) the ultrasound probe 118, such that contact between the ultrasound probe 118 and another object (e.g., the trauma patient) transmits force to the force sensor 120. In some embodiments, the force sensor 120 can be implemented using any suitable technique or combination of techniques. Additionally or alternatively, in some embodiments, the force sensor 120 can be implemented as a pressure sensor. For example, the force sensor 120 (or pressure sensor) can be resistive, capacitive, piezoelectric, etc., to sense a compressive (or tensile) force applied to the force sensor 120. In a more particular example, the force sensor 120 can be implemented using an SI-65-5 six-axis F/T Gamma transducer (available from ATI Industrial Automation, Apex, N.C.). In other embodiments, the end effector forces can be calculated using joint torques of the robot arm (e.g., deriving joint torques via measuring a current provided to at least one joint of a robot arm).

As shown in FIG. 1, the communication network 104 can facilitate communication between the mobile platform 110 of the robotic imaging system 102 and the computing device 106. In some embodiments, communication network 104 can be any suitable communication network or combination of communication networks. For example, communication network 104 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, etc. In some embodiments, communication network 104 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, etc. In some embodiments, the mobile platform 110 can implement portions of the remote trauma assessment application 134.

As shown in FIG. 1, the computing device 106 can be in communication with the haptic device 108, the communication network 104, and the mobile platform 110. Additionally, the computing device 106 can implement portions of the remote trauma assessment application 134. In general, the computing device 106 can receive positional movements from the haptic device 108, can transmit instructions to the robotic imaging system 102 (e.g., movement parameters for the robot arm 122), and can receive and present information from the mobile platform 110 (e.g., force information, ultrasound or camera images, etc.) to provide visual and/or haptic feedback to a user (e.g., a radiologist). This can allow the user to manipulate the haptic device 108 based on the feedback to control movements of the robot arm 122.

In some embodiments, the haptic device 108 can behave (or move) similarly to the robotic system 114. For example, the haptic device 108 can include a stylus (e.g., a tip-like structure that acts as the end effector of the haptic device 108), and can include segments, having a total number of degrees of freedom. In some embodiments, the stylus can have a first end and a second end, where the first end defines the tip of the stylus, which can be defined as the origin of the haptic device 108 (e.g., for the purposes of defining movements of the haptic device from an origin). Additionally, the stylus can be configured to be easily manipulatable by a user (e.g., similar to a writing utensil). In some embodiments, a size of the haptic device 108 can be a fraction (e.g., 0.5, 0.25, 0.1, etc.) of the size of the robot arm 122. In such embodiments, the haptic system 108 can implement the same or a similar number of degrees of freedom of the robot arm 122, but with sizing of the segments (and joints) of the haptic device 108 reduced by half (or a different scaling factor) compared to that of the size of the robot arm 122. Note that, the size, shape, and number of segments of the haptic device 108 can be, and often is, different than the size, shape, and number of segments of the robot arm 122. However, in some embodiments, haptic device 108 can be implemented as a scaled version of robot arm 122, with the same number of segments and same relative dimensions, but with a smaller relative size. In some embodiments, configuring the haptic device 108 to use the same coordinate system as the robotic system 114 and/or the robot arm 122 can lead to requiring that less data be acquired and/or sent, and may facilitate more accurate movements of the robot arm 122. In a particular example, the haptic device 108 can be implemented using a Geomagic Touch haptic device (available at 3D Systems, Rock Hill, S.C.). Regardless of the structure of the haptic device 108, when the haptic device 108 is manipulated, movements and/or positions of the haptic device 108 can be received by the computing device 106. In some embodiments, computing device 106 can transmit movement information and/or position information received from haptic device 108 and/or commands based on such movement information and/or position information to the mobile platform 110 to cause the robot arm 122 of the robotic system 114 to move to a specific location within the coordinate system of the robot arm 122.

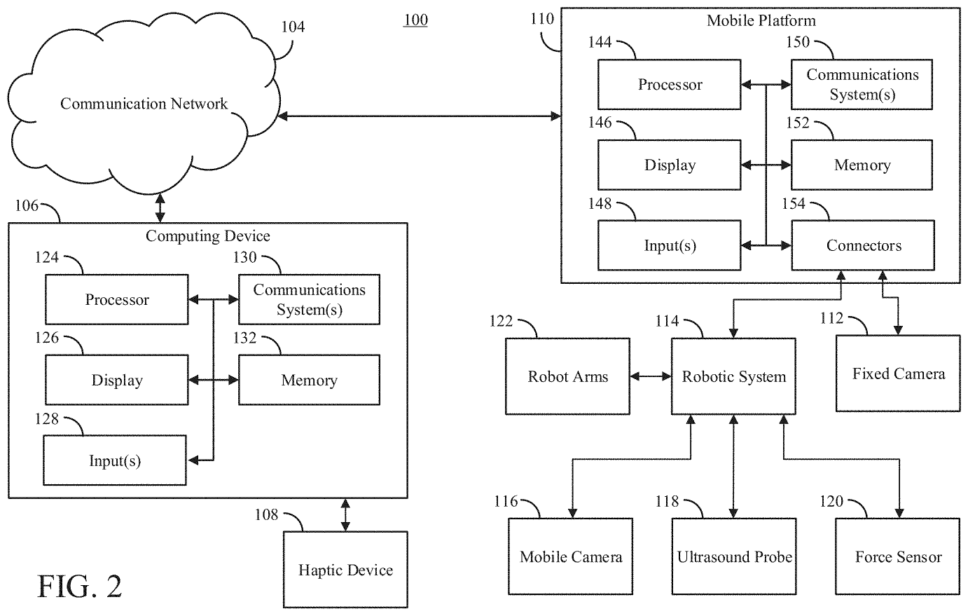

FIG. 2 shows an example of hardware that can be used to implement a computing device 106 and a mobile platform 110 shown in FIG. 1 in accordance with some embodiments of the disclosed subject matter. As shown in FIG. 2, the computing device 106 can include a processor 124, a display 126, an input 128, a communication system 130, and memory 132. The processor 124 can implement at least a portion of the remote trauma assessment application 134, which can, for example be executed from a program (e.g., saved and retrieved from memory 132). The processor 124 can be any suitable hardware processor or combination of processors, such as a central processing unit (“CPU”), a graphics processing unit (“GPU”), etc., which can execute a program, which can include the processes described below.

In some embodiments, the display 126 can present a graphical user interface. In some embodiments, the display 126 can be implemented using any suitable display devices, such as a computer monitor, a touchscreen, a television, etc. In some embodiments, the inputs 128 of the computing device 106 can include indicators, sensors, actuatable buttons, a keyboard, a mouse, a graphical user interface, a touch-screen display, etc. In some embodiments, the inputs 128 can allow a user (e.g., a medical practitioner, such as a radiologist) to interact with the computing device 106, and thereby to interact with the mobile platform 110 (e.g., via the communication network 104).

In some embodiments, the communication system 130 can include any suitable hardware, firmware, and/or software for communicating with the other systems, over any suitable communication networks. For example, the communication system 130 can include one or more transceivers, one or more communication chips and/or chip sets, etc. In a more particular example, communication system 130 can include hardware, firmware, and/or software that can be used to establish a coaxial connection, a fiber optic connection, an Ethernet connection, a USB connection, a Wi-Fi connection, a Bluetooth connection, a cellular connection, etc. In some embodiments, the communication system 130 allows the computing device 106 to communicate with the mobile platform 110 (e.g., directly, or indirectly such as via the communication network 104).

In some embodiments, the memory 132 can include any suitable storage device or devices that can be used to store instructions, values, etc., that can be used, for example, by processor 124 to present content using display 126, to communicate with the mobile platform 110 via communications system(s) 130, etc. Memory 132 can include any suitable volatile memory, non-volatile memory, storage, or any suitable combination thereof. For example, memory 132 can include RAM, ROM, EEPROM, one or more flash drives, one or more hard disks, one or more solid state drives, one or more optical drives, etc. In some embodiments, memory 132 can have encoded thereon a computer program for controlling operation of computing device 106 (or mobile platform 110). In such embodiments, processor 124 can execute at least a portion of the computer program to present content (e.g., user interfaces, images, graphics, tables, reports, etc.), receive content from the mobile platform 110, transmit information to the mobile platform 110, etc.

As shown in FIG. 2, the mobile platform 110 can include a processor 144, a display 146, an input 148, a communication system 150, memory 152, and connectors 154. In some embodiments, the processor 144 can implement at least a portion of the remote trauma assessment application 134, which can, for example be executed from a program (e.g., saved and retrieved from memory 152). The processor 144 can be any suitable hardware processor or combination of processors, such as a central processing unit (“CPU”), a graphics processing unit (“GPU”), etc., which can execute a program, which can include the processes described below.

In some embodiments, the display 146 can present a graphical user interface. In some embodiments, the display 146 can include any suitable display devices, such as a computer monitor, a touchscreen, a television, etc. In some embodiments, the inputs 148 of the mobile platform 110 can include indicators, sensors, actuatable buttons, a keyboard, a mouse, a graphical user interface, a touch-screen display, and the like. In some embodiments, the inputs 148 allow a user (e.g., a first responder) to interact with the mobile platform 110, and thereby to interact with the computing device 106 (e.g., via the communication network 104).

As shown in FIG. 2, the mobile platform 110 can include the communication system 150. The communication system 150 can include any suitable hardware, firmware, and/or software for communicating with the other systems, over any suitable communication networks. For example, the communication system 150 can include one or more transceivers, one or more communication chips and/or chip sets, etc. In a more particular example, communication system 150 can include hardware, firmware, and/or software that can be used to establish a coaxial connection, a fiber optic connection, an Ethernet connection, a USB connection, a Wi-Fi connection, a Bluetooth connection, a cellular connection, etc. In some embodiments, the communication system 150 allows the mobile platform 110 to communicate with the computing device 106 (e.g., directly, or indirectly such as via the communication network 104).

In some embodiments, the memory 152 can include any suitable storage device or devices that can be used to store instructions, values, etc., that can be used, for example, by processor 144 to present content using display 146, to communicate with the computing device 106 via communications system(s) 150, etc. Memory 152 can include any suitable volatile memory, non-volatile memory, storage, or any suitable combination thereof. For example, memory 152 can include RAM, ROM, EEPROM, one or more flash drives, one or more hard disks, one or more solid state drives, one or more optical drives, etc. In some embodiments, memory 152 can have encoded thereon a computer program for controlling operation of the mobile platform 110 (or computing device 106). In such embodiments, processor 144 can execute at least a portion of the computer program to present content (e.g., user interfaces, graphics, tables, reports, etc.), receive content from the computing device 106, transmit information to the computing device 106, etc.

In some embodiments, the connectors 154 can be wired connections, such that the fixed camera 112 and the robotic system 114 (e.g., including the mobile camera 116, the ultrasound probe 118, and the force sensor 120) can communicate with the mobile platform 110, and thus can communicate with the computing device 106 (e.g., via the communication system 150 and being directly, or indirectly, such as via the communication network 104). Additionally or alternatively, the fixed camera 112 and/or the robotic system 114 can send information to and/or receive information from the mobile platform 110 (e.g., using the connectors 154, and/or the communication systems 150).

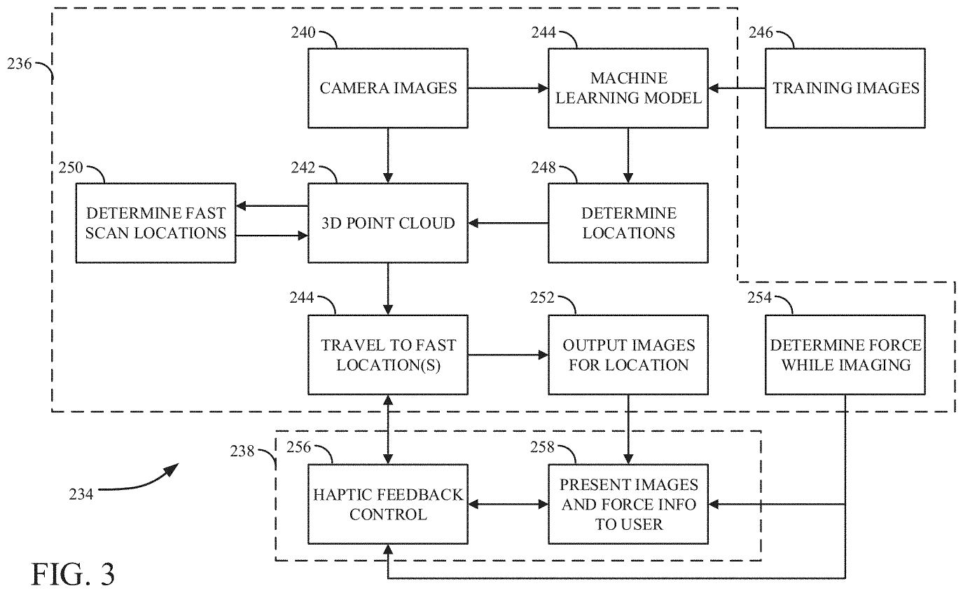

FIG. 3 shows a schematic illustration of a remote trauma assessment flow 234 in accordance with some embodiments of the disclosed subject matter. The remote trauma assessment flow 234 can be used to implement at least a portion of the remote trauma assessment application 134 that can be implemented using the trauma assessment system 100. In some embodiments, the trauma assessment flow 234 can include an autonomous portion 236 and a non-autonomous portion 238. The autonomous portion 236 of the trauma assessment flow 234 can begin at, and include, acquiring camera images 240. Acquiring camera images 240 can include autonomously acquiring one or more 3D images of the patient (e.g., via the mobile camera 116). In some embodiments, the robotic system 114 can perform sweeping motions over the patient to record several 3D images (and/or other depth data), which can be combined to generate point cloud data. For example, the robotic system 114 can perform a 3D scan by acquiring 3D images with the mobile camera 116 (e.g., a RGB-D camera) in 21 pre-programmed positions around the patient. In some embodiments, for each sweeping motion, the robotic system 114 can perform a semicircular motion around the patient at a distance of 30 centimeters (“cm”) with the mobile camera 116 facing toward the patient. Additionally in some embodiments, using the fixed camera 112, one or more two-dimensional (“2D”) color images (e.g., red, green, and blue (“RGB”) images) can be acquired during this process for detection and localization of the umbilicus, wounds, and/or bandages.

After acquiring camera images 240, the flow 234 can include generating a 3D point cloud 242. The previously acquired images from the mobile camera 116 (e.g., camera images 240) can be used to generate the 3D point cloud 242. For example, the mobile platform 110 (or the computing device 106) can perform a point cloud registration using the 3D images (depicting multiple scenes) to construct a 3D model using any suitable technique or combinations of techniques. For example, the mobile platform 110 can use the Iterative Closest Point (“ICP”) algorithm. In such an example, the ICP algorithm can be implemented using the point cloud library (“PCL”), and pointmatcher, and can include prior noise removal with a PCL passthrough filter. In some embodiments, a point cloud color segmentation can be applied to extract a point cloud corresponding to the patient from the reconstructed scene, for example by removing background items such as a table and a supporting frame.

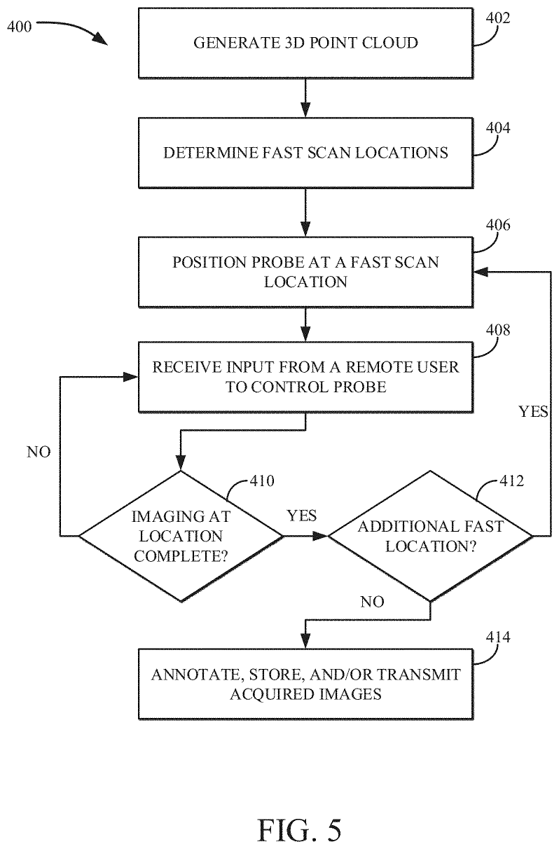

After acquiring camera images 240 and/or generating 3D point cloud 242, the flow 234 can include inputting images into the machine learning model 244. The machine learning model 244 can classify one or more portions of the inputted image(s) into various categories, such as umbilici, bandages, wounds, mammary papillae (sometimes referred to as a nipple), skin, etc. Additionally or alternatively, in some embodiments, the machine learning model 244 can identify, and/or output a segmented image of a correctly identified location or region of interest within the image (e.g., an umbilicus). In some embodiments, the identified location(s) 248 (or region of interest) for each image can be mapped to the 3D point cloud 242 (or model) (e.g., which can later be used to avoid the locations during ultrasound imaging). In some embodiments, any suitable type of location can be identified, such as, an umbilicus, a bandage, a wound, and a mammary papillae. In some embodiments, such features can be identified as reference points and/or as points that should not be contacted during ultrasound imaging (e.g., wounds and bandages). In some embodiments, identification and segmentation of the umbilicus can be used to provide an anatomical landmark for finding the FAST scan locations, as described below (e.g., in connection with FIG. 6).

In some embodiments, the machine learning model 244 can be trained using training images 246. The training images 246 can include examples of correctly identified images that depict the classes described above (e.g., umbilicus, a wound, a bandage, a mammary papilla, etc.). In some embodiments, the machine learning model 244 can be trained to classify images as including an example of a particular class and/or can output a mask or other information segmenting the image to identify where in the image the example of the class was identified. In some embodiments, locations of objects identified by the machine learning model 244 can be mapped to the 3D point cloud 242 at determine locations 248 of flow 234. In some embodiments, prior to inputting a particular image into the machine learning model 244, the location of the image can be registered to the 3D point cloud 242 such that the location within the image at which an object (e.g., a wound, an umbilicus, a bandage, etc.) is identified can be mapped to the 3D point cloud. In some embodiments, flow 234 can use a region identified within an image (e.g., the umbilicus region) to map the region of the image to the 3D point cloud 242 at determine locations 248.

In some embodiments, at determine FAST scan locations 250 of flow 234, the FAST scan locations can be determined relative to the 3D point cloud of the patient. In some embodiments, the mobile platform 110 (and/or the computing device 106) can use an anatomical landmark to determine the dimensions of the 3D point cloud 242. For example, the location of umbilicus on the subject can be determined and used as the anatomical landmark (e.g., being previously calculated at determine locations 248) to calculate the FAST scan positions. In some embodiments, the FAST scan positions of the subject can be derived by scaling an atlas patient for which FAST scan locations have already been determined using the ratio of the dimensions of atlas and the patient. For example, the atlas can be generated using a 3D model of a CT scan of an example patient having similar proportions to the patient with hand segmented (or identified) FAST locations provided by an expert radiologist (e.g., the atlas being annotated with FAST locations). In some embodiments, scaling of the atlas can be carried out by first determining the width and height of the patient based on the 3D point cloud 242. For example, the width of the patient can be derived by projecting the 3D point cloud 242 into the x-y plane and finding the difference between the maximum y and the minimum y (e.g., the distance between). As another example, the height of the patient can be derived by projecting the 3D point cloud 242 into the x-z plane and finding the difference between the maximum z and the minimum z (e.g., the distance between). As yet another example, a length variable associated with the patient can be derived by measuring the distance from the umbilicus to the sternum of the atlas. In some embodiments, the mobile platform 110 (or computing device 106) can use the known length, width, and/or height, and FAST scan positions of the atlas to calculate the FAST scan position on the subject using the following relationship:

$$[ \begin{matrix}{X}_{p} \\ {Y}_{p} \\ {Z}_{p}\end{matrix} ] = [ \begin{matrix}\frac{{l}_{p}}{{l}_{a}} & 0 & 0 \\ 0 & \frac{{w}_{p}}{{w}_{a}} & 0 \\ 0 & 0 & \frac{{h}_{p}}{{h}_{a}}\end{matrix} ] [ \begin{matrix}{X}_{a} \\ {Y}_{a} \\ {Z}_{a}\end{matrix} ] \qquad ( 1 )$$

where, Xp, Yp, and Zp are the coordinates of a FAST scan position (e.g., FAST scan position 1) on the subject, Xa, Ya, Za are the coordinates of a FAST scan position on the atlas (which are known), lp, wp, and hp are the length, width, and height associated with the patient's torso, and la, wa, and ha are the length, width, and height associated with the atlas. In some embodiments, the length between the umbilicus and sternum can be estimated using EQ. (1). Note that in some embodiments, an approximation of the ratio of lp to la can be derived from the other variables. In some embodiments, each of the FAST scan locations can be determined (e.g., at 242 using EQ. (1) for each remaining location), and each FAST scan location can be mapped onto the 3D point cloud 242.

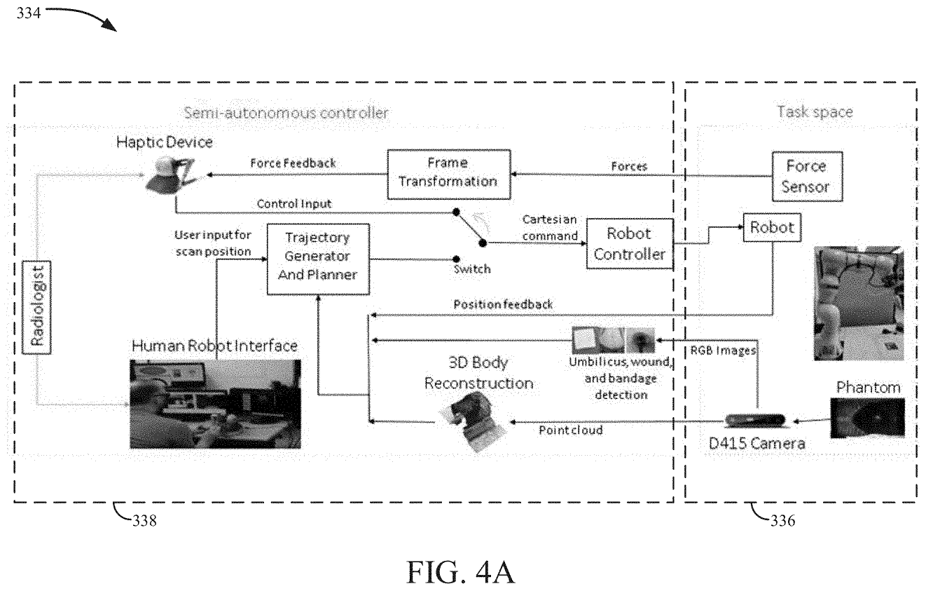

In general, the location of the xiphoid process can be difficult to determine visually from image data acquired by cameras 112 and/or 116. In some embodiments, the umbilicus can be used as visual landmark in combination with the measured dimensions of the patient's torso in the 3D point cloud to estimate the FAST locations. In some embodiments, CT scan images of one or more anonymized atlas patients can be used to identify a ratio (e.g., R₁ in EQS. (2) and (3) below) between the length of a person's torso to the distance between the umbilicus and the 4th FAST position (e.g., the person's xiphoid). A ratio (R₂ in EQS. (2) and (3) below) between C (e.g., the entire length of the torso) and the distance of 3^{rd} FAST scan region (e.g., the abdomen) from the umbilicus can be calculated. The distance D can represent the distance from the 4th FAST position to the 1^{st} and 2^{nd} FAST positions along the y-axis. Another relation R₃ (in EQS. (2) and (4)) between C and D can be calculated. Distance A can be the distance from the umbilicus to the fourth fast scan location along the y-axis (see FIG. 4B, panel (c)). Distance B can be the distance from the umbilicus to the third fast scan location along the y-axis (see FIG. 4B, panel (c)). The width, length, and height of the subject, along the x, y, and z axes respectively, can be estimated from the reconstructed 3D model (e.g., using the procedure above and EQ. (1)). The FAST scan coordinates for the subject can be calculated using the following relationships below in EQS. (2)-(5):

$$\begin{matrix}[ \begin{matrix}{R}_{1} \\ {R}_{2} \\ {R}_{3}\end{matrix} ] = 1 \text{/} C [ \begin{matrix}A \\ B \\ A - D\end{matrix} ] & ( 2 ) \\ [ \begin{matrix}{X}_{1} \\ {X}_{2} \\ {X}_{3} \\ {X}_{4}\end{matrix} ] = [ \begin{matrix}{X}_{u} + E \\ {X}_{u} - E \\ {X}_{u} \\ {X}_{u}\end{matrix} ] & ( 3 ) \\ [ \begin{matrix}{Y}_{1} \\ {Y}_{2} \\ {Y}_{3} \\ {Y}_{4}\end{matrix} ] = {Y}_{u} + [ \begin{matrix}{R}_{3} C \\ {R}_{3} C \\ - {R}_{2} C \\ {R}_{1}\end{matrix} ] & ( 4 ) \\ [ \begin{matrix}{Z}_{1} \\ {Z}_{2} \\ {Z}_{3} \\ {Z}_{4}\end{matrix} ] = H [ \begin{matrix}0.5 \\ 0.5 \\ 1 \\ 1\end{matrix} ] & ( 5 )\end{matrix}$$

In EQS. (3), (4), and (5) Xᵢ, Yᵢ, Zᵢ for iϵ1, 2, 3, 4 can be the coordinates of the respective FAST scan positions while Xᵤ, Yᵤ, Zᵤ can be the coordinates of the detected umbilicus in world frame, E can be the width of the subject (e.g., wp in EQ. (1)), and H can be the height of the patient (e.g, hp in EQ. (1)). In some embodiments, mean values for the atlases for R1, R2, and R3 can be 0.29, 0.22, and 0.20, respectively.

In some embodiments, the robotic system 114 can move the ultrasound probe 118 to a specific location corresponding to a FAST location, using the FAST scan locations been determined at 250. In some embodiments, the mobile platform 110 can receive a user input (e.g., from the computing device 106) that instructs the robotic system 114 to travel to a particular FAST scan location (e.g., the first FAST scan location). In some embodiments, the coordinate of the fast scan location (e.g., Xp1, Yp2, Zp3) can be utilized by the robotic system 114 as an instruction to travel to the first coordinate location. Additionally or alternatively, the FAST scan coordinate location can define a region surrounding the determined fast scan location (e.g., a threshold defined by each of the coordinates, such as a percentage), so as to allow for preforming a FAST scan near a location (e.g., where the specific coordinate is impeded by a bandage or wound). For example, if the first coordinate location is impeded (e.g., with a bandage), the robotic system 114 can utilize a location within a threshold distance of that coordinate, to perform a FAST scan near that region on the patient.

In some embodiments, after moving the ultrasound probe 118 to the FAST scan location (specified by the instructed coordinate or region), the robotic system 114 can determine whether or not contact between the ultrasound probe 118 and the subject can be sufficiently established for generating an ultrasound image (e.g., of a certain quality). For example, the mobile platform 110 can receive a force (or pressure) value from the force sensor 120 (e.g., at determine force while imaging 254). In such an example, the mobile platform 110 can determine whether or not the ultrasound probe 118 is in contact with the patient based on the force value. In a more particular example, if the force reading is less than (and/or based on the force reading being less than) a threshold value (e.g., 1 Newton (“N”)), the robotic system 114 can move the ultrasound probe axially (e.g., relative to the ultrasound probe surface) toward the patient's body until the force reading reaches (and/or exceeds) a threshold value (e.g., 3 N). In some embodiments, after the robotic system 114 places the ultrasound probe 118 in contact with the patient with sufficient force, the mobile platform 110 can begin generating and/or transmitting ultrasound data/images. In some embodiments, the force sensor value from the force sensor 120 can be calibrated prior to movement to a FAST location.

In some embodiments, after the robotic system 114 travels to the particular FAST scan location, the robotic system 114 can cease movement operations (e.g., bypassing the axial movement and corresponding force reading) until user input is received at the mobile platform 110 (e.g., sent by the computing device 106) to initiate the haptic feedback controller 256. Additionally, in some embodiments, in response to the user input being received by the mobile platform 110, the mobile platform 110 can provide (e.g., by transmitting) ultrasound images for the specific location 252 to the computing device 106, and/or can provide the force value from the force sensor 120 to the computing device 106. In such embodiments, the computing device 106 can present images and force to user 258 (e.g., digital images, ultrasound images, and feedback indicative of a force with which the ultrasound probe 118 is contacting the patient). In some embodiments, the ultrasound images and the force values (e.g., time based force values), can be assessed by a user (e.g., a radiologist), and the user can adjust the orientation of the haptic device 108, thereby manipulating the orientation and position of the ultrasound probe 118 (e.g., via the robotic system 114). For example, when the user initiates the haptic feedback control 256, movements of the stylus (e.g., the end effector) of the haptic device 108 can be translated into commands for the robotic system 114.

In some embodiments, the mobile platform 110 can receive force information from the force sensor 120, and can transmit the force information to the computing device 106 and/or the haptic device 108. In some embodiments, the force information can be a decomposition of the normal force into magnitude and directional components along each dimensional axis (e.g., x, y, and z directions), based on the position and orientation of the robot arm 122. Alternatively, the normal force value can be transmitted and decomposed by the computing device 106 into magnitude and directional components along each three dimensional axis. In some embodiments, the haptic device 108 can use the force values (e.g., along each axis) to resist movement (e.g., of one or more particular joints and/or segments of the haptic device 108), based on the magnitude and direction of the force (e.g., from the force sensor 120). In some embodiments, the magnitude of the forces can be proportional to the force value provided by the force sensor 120 (e.g., scaled down, or up, appropriately).

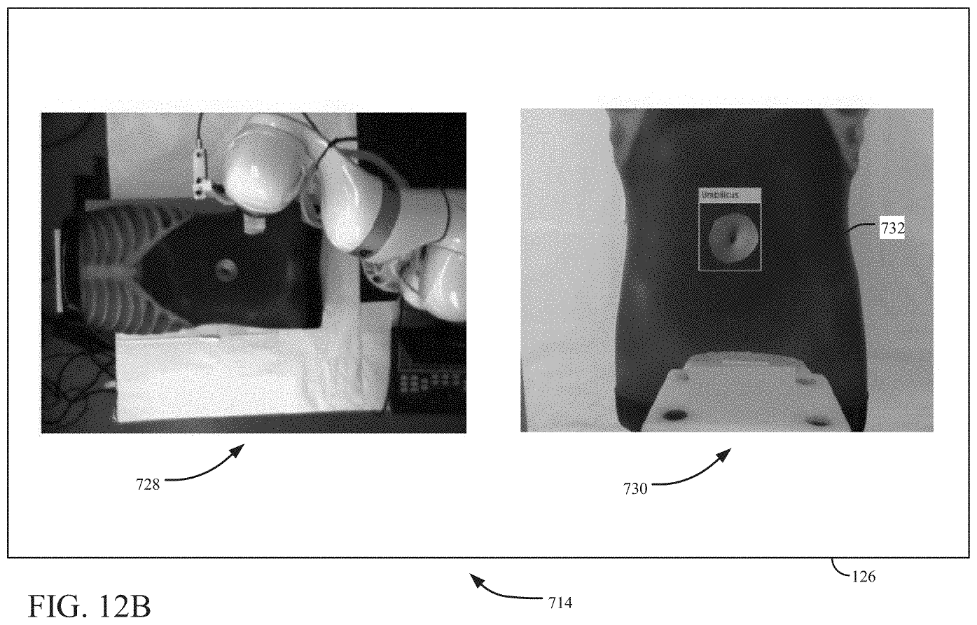

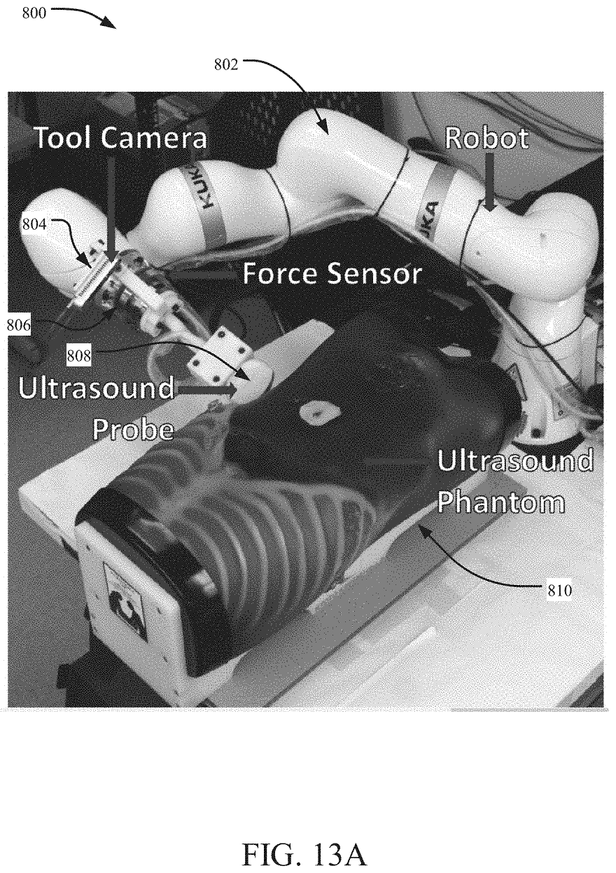

In some embodiments, the forces acting along the normal axis of the EE of the robot arm 122 (e.g., the zR axis described below in connection with FIG. 15) can be received from the force sensor 120 by the mobile platform 110. The force value can be received by the computing device 106, and the computing device 106 can reflect the force value along the normal axis through the tip of the stylus of the haptic device 108 (e.g., the yH axis described below in connection with FIGS. 14A and 15) to cause the haptic device 108 to resist movement along the yH axis. In some embodiments, the haptic device 108 can resist movement along the normal axis of the stylus of the haptic device 108 by applying appropriate torques to the joints of the haptic device 108.

In some embodiments, position guiding, rate guiding, or combinations thereof, can be used to control the EE of the robot arm 122 (e.g., the ultrasound probe 118). For example, in some embodiments, computing device 106 and/or mobile platform 110 can receive movement information from the haptic device 108, and using the movement information from the haptic device 108, can generate and provide movement commands for the robot arm 122 as increments to the robot arm 122. The movement commands can be defined from the initial Cartesian pose of the robot arm 122 (e.g., the pose prior to the incremental movement, prior to a first incremental movement, etc.), such as in the coordinate system shown in FIG. 15, to another position. In some embodiments, the initial Cartesian pose can be defined as the pose of the robot arm 122 after autonomously driving to a FAST scan location.

In some embodiments, forward kinematics for translations of the haptic device 108 can be calculated with respect to the origin shown in FIG. 14A using any suitable technique or combination of techniques. For example, Phantom Omni driver available from 3D Systems can be used to determine forward kinematics for the haptic device 108 using the tip of the stylus of the haptic device 108 as the origin and mean positions of each joint of the haptic device 108. In some embodiments, movements in the coordinate system of the haptic device 108 (e.g., defined by axes x_{H}, y_{H}, z_{H}, where the axes intersect at the tip of the stylus) can be transformed and/or scaled (e.g., by computing device 106 and/or mobile platform 110) into a coordinate system of the robotic imaging system 114 and/or the robot arm 122 (e.g., defined by axes x_{R}, z_{R}, y_{R} axes, where the axes intersect at the end of the ultrasound probe 118). The transformed coordinates (or movement instructions) can be transmitted to the mobile platform 110 and/or the robot arm 122 to instruct the robot arm 122 to move to a new location based on input provided to the haptic device 108. In some embodiments, the mobile platform 110 can incrementally transmit movement instructions to move the robot arm 122, from an original location (e.g., the initial Cartesian pose) to a final location. For example, a series of stepwise movement instructions beginning from the original location and incrementally spanning to the final location can be sent to the robot arm 122.

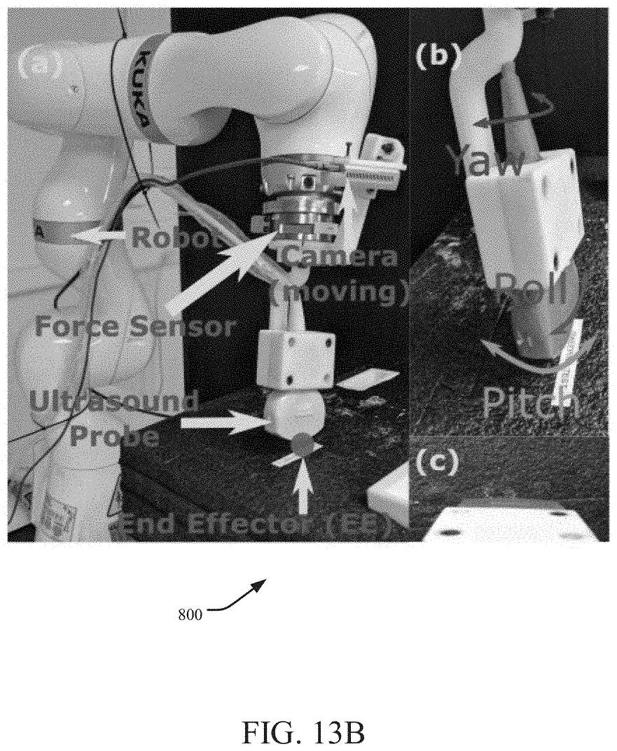

In some embodiments, the mobile camera 116 can be rigidly mounted on the robot arm 122, such that there is a fixed transform between the camera and the EE of the robot arm 122. For example, as described below in connection with FIG. 16, x_{C}, y_{C}, and z_{C} on the upper left corner of the image from the mobile camera 116 can correspond to x_{R}, y_{R}, and z_{R}, respectively in the EE frame of reference of the robot arm 122.

In some embodiments, computing device 106 and/or mobile platform 110 can transform the initial position of the robot arm 122 for each FAST scan location into the instantaneous EE frame. Additionally, in some embodiments, computing device 106 and/or mobile platform 110 can transform the FAST scan location into the world frame of the robotic system 114 (e.g., based on the origin of the robot arm 122, which can be the base of the most proximal segment of the robot arm 122). In some embodiments, robot arm 122 can inhibit the position and orientation of the EE from being changed simultaneously. Thus, in some embodiments, commands for changing the position and orientation of the EE of the robot arm 122 can be decoupled such that the position and orientation are limited to being changed independently (e.g., one at a time), which may eliminate any unintentional coupling effects. In some embodiments, orientation commands from the haptic device 108 can be determined using only the displacement of joints 4, 5, and 6 for roll, pitch, and yaw respectively (e.g., from their center positions) of the haptic device 108. For example, joints 4, 5, and 6 can be the most distal joints of the haptic device 108 as described below in connection with FIGS. 14A and 14B.

In some embodiments, an actuatable button on the stylus of the haptic device 108 can be used to control whether position commands or orientation commands are received by the computing device 106, and subsequently relayed to the mobile device 110 to instruct the robot arm 122 to move. For example, the actuatable button can be normally in a first position (e.g., open, un-depressed, etc.), which causes the computing device 106 to only receive translation commands from the haptic device 108 (e.g., while keeping the orientation of the EE of the robot arm 122 constant). In such an example, when the actuatable button is in a second position (e.g., closed, depressed, etc.), the computing device 106 can receive only orientation commands from the haptic device 108 (e.g., while keeping the position of the EE of the robot arm 122 constant).

In some embodiments, the mobile device 110 can automatically perform an indexing operation, allowing the user to re-center the haptic device 108 for translations or orientations by keeping one of them constant and changing the other, while refraining from sending any commands to the robot arm 122. The initial Cartesian pose of the robot arm 122 (e.g., using the EE coordinate system of the robot arm 122) can be updated with its current Cartesian pose every time the state of the actuatable button changes. In some embodiments, the operator can have control over the yaw (e.g., joint 7), of the robot arm 122, using joint 6 of the haptic device 108, for the slave (e.g., the robot arm 122), such that the most distal joint of the device 108 controls the most distal joint of the robot arm 122. In some embodiments, the position control scheme can be mathematically described using the following relationship:

$${X}_{{R}_{EE}} = {H}_{w}^{T} {X}_{{R}_{{EE}_{0}}} + {K}_{{P}_{1}} {X}_{H} \qquad ( 6 )$$

where X_{R}_{_{EE}}=[x y z 1]^{T} can include the new x-y-z coordinates of the EE of the robot arm 122 in the current EE frame, H_{w}^{T}∈ [symbol] ^{4×4} can be the homogenous transformation matrix from the robot world frame to the current EE frame,

$${X}_{{R}_{{EE}_{0}}} = {[ \begin{matrix}{x}_{0} & {y}_{0} & {z}_{0} & 1\end{matrix} ]}^{T}$$

can include the initial positions of the EE in the world frame, K_{P}_{₁}=diag[kₚ₁, kₚ₂, Kₚ₃, 1]>0 can be the controller gains, and X_{H}=[x_{H} y_{H} z_{H} 1]^{T} can be the displacements of the haptic device 108 relative to the origin of the haptic device 108 (e.g., defined at the tip of the stylus of the haptic device 108, with all of the joints of the haptic device 108 at their initial starting positions).

In some embodiments, a rate control scheme can be mathematically described using the following relationship:

$${θ}_{{R}_{EE}} = {R}_{w}^{T} {θ}_{{R}_{{EE}_{0}}} + {K}_{{P}_{2}} {θ}_{H} \qquad ( 7 )$$

where, θ_{R}_{_{EE}}∈ [symbol] ³ can be the new roll, pitch, and yaw angles of the EE of the robot arm 122 in the current EE frame, R_{w}^{T}∈ [symbol] ^{3×3} can be the rotation matrix from the robot world frame to the EE frame,

$${θ}_{{R}_{{EE}_{0}}}$$

∈ [symbol] ³ can be the initial roll, pitch, and yaw angles of the EE in the world frame, K_{P}_{₂}=diag[kₚ₄, kₚ₅, kₚ₆]>0 can be the controller gains, θ_{H}∈ [symbol] ³ can be the displacements of joints J4, J5, and J6 of the haptic device 108 from their respective mean positions.

In some embodiments, computing device 106 and/or mobile platform 110 can transform the Cartesian pose formed from x_{R}_{_{EE}} and θ_{R}_{_{EE}} into the world frame of the robot arm 122, then convert into the joint space of the robot arm 122 using inverse kinematics, before instructing the robot arm 122 to move.

In some embodiments, the EE position feedback from the robot (e.g., the ultrasound probe 118) can be used, in addition to the position commands from the haptic device 108 multiplied by a scaling factor (e.g., 1.5), to determine the EE reference positions of the robot in the task space. This can be done by adding new reference positions read from the displacement of the haptic device 108 to command the final EE position of the robot in a Cartesian task-space as shown in EQ. (8) below.

$$[ \begin{matrix}X \\ Y \\ Z\end{matrix} ] = {R}_{w}^{T} [ \begin{matrix}{Init}_{x} \\ {Init}_{y} \\ {Init}_{z}\end{matrix} ] + [ \begin{matrix}{H}_{x} \\ {H}_{y} \\ {H}_{z}\end{matrix} ] \qquad ( 8 )$$

The forward kinematics for positions of the haptic device 108 can be calculated with respect to the origin (e.g., the starting position of the tip of the stylus) through the phantom omni driver. The X, Y, and Z coordinates can be the new coordinates of the EE. Initₓ, Init_{y}, Init_{z} can be the initial positions of the EE (e.g., the ultrasound probe 118) for that scan position. Hₓ, H_{y}, H_{z} can be the positions of the tip of the stylus of the haptic device 108 relative to an origin of the haptic device 108 (e.g., the tip of the stylus at a point in time, such as after the ultrasound probe 118 moves to the particular fast scan location).

In some embodiments, the stylus of haptic device 108 can be oriented using, the roll, pitch, and yaw (e.g., see FIGS. 14A and 14B). The stylus of the haptic device 108 can be controlled using three individual wrist joints of the haptic device 108, which each control a degree of freedom of the stylus of the haptic device 108. Additionally, the rate of change of each orientation can be directly proportional to the deviation of each axis (see, e.g., J4, J5, J6 as shown in FIG. 14B) from its mean position. A dead-zone of −35 degrees to +35 degrees in roll and yaw, and −20 degrees to +20 degrees in pitch axis can created to avoid accidental change in EE orientation (e.g., the dead zone preventing movement within a particular dead zone). The rate control scheme can be mathematically represented below in EQ. (9).

$$[ \begin{matrix}{\dot{θ}}_{r} \\ {\dot{θ}}_{p} \\ {\dot{θ}}_{y}\end{matrix} ] = [ \begin{matrix}{K}_{1} \\ {K}_{2} \\ {K}_{3}\end{matrix} ] [ \begin{matrix}{H}_{o 4} \\ {H}_{o 5} \\ {H}_{o 6}\end{matrix} ] \qquad ( 9 )$$

In EQ. (9), θ^{⋅}ᵣ, θ^{⋅}ₚ, and θ^{⋅}_{y} are the roll, pith, and yaw rates of the EE (of the robot arm) in the world frame. K1, K2, and K3 can be controller gains. Ho4, Ho5, Ho6 can be wrist joint angles of the haptic device 108 (see, e.g., FIG. 14B). The rate control scheme can decouple the orientations from each other, which can cause the user to move one axis at a time, and ensure that the orientation of the haptic device is calibrated with what the user sees in the tool camera as soon as roll, pitch, and yaw angles J4, J5, and J6 of the haptic device can be brought inside their respective dead zones.

In some embodiments, a hybrid control scheme can be used to control the robot arm 122. For example, the hybrid control scheme can use a combination of position and rate guiding modes. In some embodiments, the translations can be controlled using the components and techniques described above (e.g., using EQS. (6) or (8)). In some embodiments, the rate control can be used as a guiding scheme during the manipulation of the EE of the robot arm 122 while the position is maintained (e.g., during orientation commands). The roll, pitch, and yaw rate of the EE of the robot arm 122 (e.g., the three most distal joints of the robot arm 122) can be controlled using individual wrist joints (e.g., J4, J5, and J6) of the haptic device 108. The rate of change of each orientation of the haptic device 108 can be directly proportional to the deviation of each axis of the respective joint (e.g., the angles of J4, J5, J6) relative to their corresponding mean position. In some embodiments, a dead-zone of −35 degrees to +35 degrees can be used for the roll and yaw of the haptic device 108, a dead zone of −20 degrees to +20 degrees can be used for the pitch of the haptic device 108. The respective dead zones can avoid accidental change in EE orientation of the robot arm 122.

In some embodiments, a rate control scheme without the dead zones can be mathematically represented using the following relationship:

\dot{θ}R_{EE}=K_{θ}θ_{H} (10)

where, \dot{θ}_{R}_{_{EE}}∈ [symbol] ³ can be the roll, pitch, and yaw rates of the EE in the world frame, K_{θ}=diag[k_{θ1}, k_{θ2}, k_{θ3}]>0 can be the controller gains, θ_{H}∈ [symbol] ³ can be the displacements of joints J4, J5, and J6 of the haptic device 108 from their respective mean positions, \dot{θ}_{R}_{_{EE}} can be integrated at a constant loop rate of 1 kHz to obtain θ_{R}_{_{EE}}. In some embodiments, computing device 106 and/or mobile platform 110 can transform the Cartesian pose formed from x_{R}_{_{EE}} and θ_{R}_{_{EE}} can first be into the world frame of the robot arm 122, then into the joint space of the robot arm 122 using inverse kinematics, before instructing the robot arm 122 to move.

In some embodiments, the rate control scheme described above in connection with EQS. (7), (9), and (10) can decouple orientations from each other, which may implicitly make the user move one orientation axis at a time and ensure that the orientation of the haptic device 108 is calibrated with what the user sees in the tool camera (e.g., the mobile camera 116) as roll, pitch, and yaw angles J4, J5, and J6 of the haptic device 108 are brought inside their respective dead zones.

In some embodiments, as the user (e.g., a radiologist) manipulates the haptic device 108, the user can be presented with ultrasound images and force values at 258. The ultrasound images and the force values can be transmitted to the computing device 106 (e.g., via the communication network 104) in real-time. In some embodiments, real-time feedback provided at 258 can assist a user in determining how to adjust the position (and/or orientation) of the stylus of the haptic device 108 to thereby move the ultrasound probe 118 of the robotic system 114. Generally, the force values from the force sensor 120 can be forces acting normal to the scanning surface of the ultrasound probe 118 (e.g., because the quality of the ultrasound image is only affected by normal forces, and not lateral forces applied by the ultrasound probe 118). In some embodiments, the force value from the force sensor 120 can be calibrated to adjust for gravity.

In some embodiments, a soft virtual fixture (“VF”) can be implemented (e.g., as a feature on a graphical user interface presented to the user) to lock the position of the EE of the robotic system 114, while implementing (only) orientation under certain conditions (e.g., as soon as a normal force greater than a threshold of 7N was received). For example, the user can make sweeping scanning motions while the robotic system 114 maintains the ultrasound probe 118 in stable and sufficient contact with the patient. For example, when the virtual fixture is initiated, the virtual fixture can prevent any translation except in the +z_{R} axis (e.g., the axis normal to the ultrasound probe 118 and away from the patient). In some embodiments, to continue scanning and deactivate the virtual fixture, the user can be required to move the ultrasound probe 118 away until the ultrasound probe 118 is no longer in contact with the patient (e.g., via the haptic device 108). Additionally, in some embodiments, a hard virtual fixture can be used to cut the system off (e.g., cease operation of the robotic system 114) if (and/or based on) the magnitude of forces acting on the patient exceeds 12N (or any other suitable force sensor values).

In some embodiments, when the soft VF is initiated, the soft VF can “lock” the EEs of the robot arm 122 to inhibit translation motion, while still allowing orientation control, when a normal force value received by a suitable computing device is greater than a threshold value. In some embodiments, the soft VF can help limit the forces applied to the patient while keeping the probe stable during sweeping scanning motions. In some embodiments, the force threshold for the soft VF can be set to about 3N (e.g., which can be determined experimentally based on ultrasound image quality).

In some embodiments, force feedback models can be used prior to contact with the patient. For example, computing device 106 and/or mobile platform 110 can generate an artificial potential field(s) (“APF”), which can be defined as emerging from the center of a wound and applying a force in a direction away from the wound with a strength that diminishes with distance from the wound (e.g., the field can decrease inversely to the square of the distance). For example, the APF can be used to calculate a force to apply along a direction from the wound to the EE based on the current distance between the EE and the wound. In some embodiments, APFs can be are generated in the shape of a hemisphere with a radius 2 cm or greater than the radius of the identified wound. For example, the radius of the APF shown in FIG. 4B is set to be at least 2 cm greater than the region identified as a gunshot wound in panel (f). In some embodiments, computing device 106 and/or mobile platform 110 can cause a force to be exerted on the origin of the haptic device 108 and/or the EE of the robot arm 122 in proportion to the strength of the APF to help guide the probe away from the wounds. In some embodiments, the mobile platform 110 and/or robotic system 114 can calculate a virtual force to be provided as feedback via the haptic device 108 based on the location of the EE and the field strength of the APF at that location. In such embodiments, the mobile platform 110 can communicate the virtual force to the computing device 106, which can provide a force (e.g., based on the virtual force and/or a force value output by the force sensor 120) to be used by the haptic device 108 during manipulation of the haptic device 108 by a user. Additionally or alternatively, in some embodiments, the mobile platform 110 and/or the robotic system 114 can provide a virtual fixture at a fixed distance from a contour of the wound (e.g., any suitable distance from 0.1 cm to 0.5 cm) that inhibits movement of the EE into the space defined by the virtual fixture.

In some embodiments, a user can provide input to instruct the robotic system 114, via a user input on the computing device 106, to travel to a next FAST location (e.g., when the user deems that ultrasound images gathered at a particular FAST location can be sufficient for diagnostic purposes (e.g., above an image quality level)). At the next FAST location, flow 234 can repeat the actions of presenting images and force to the user at 258, and using haptic feedback control at 256. After all of the desired FAST locations have been imaged with the ultrasound probe 118, the radiologist (or other user, or computing device 106) can analyze the ultrasound images/data for a diagnostic determination. In some embodiments, the radiologist can communicate with the first responders (e.g., via the communication device 106 communicating with the mobile platform 110) to convey information about the diagnosis to the first responders. Examples of instructions can be a graphical image on the display (e.g., the display 146), audible instructions through the inputs 148 (e.g., a microphone), etc. This way, the first responders can implement life-saving techniques while in transit to the trauma center (e.g., hospital).

FIG. 4 shows an example of another schematic illustration of a remote trauma assessment flow 334, in accordance with some embodiments of the disclosed subject matter. In some embodiments, the remote trauma assessment flow 334 can be a specific implementation of the remote trauma assessment flow 234, which can be implemented using the trauma assessment system 100. The trauma assessment flow 334 can be similar to the trauma assessment flow 234, thus what pertains to the trauma assessment flow 234 also pertains to the trauma assessment 334. Within the trauma assessment flow 334, the task space 336 can be the portion where data is generated, while the portion 338 can be the portion where commands are determined. For example, the task space 336 can include the mobile platform 110 capturing images (3D imaging data, and 2D images of the subject), moving the robot arm, sensing the force from the force sensor, and generating ultrasound images. As another example, the computing device 106 can detect a region of interest (e.g., the umbilicus, a wound, a bandage, etc.) within an image, construct of a 3D model of the subject, display of forces, and display of ultrasound images. Additionally, the movement of the haptic device (which controls the robot) can be manipulated by a user, and the initiation of the user selecting what location for the robot to move to can be transmitted to the robotic system via a user input (e.g., indicated by the switch in the lower position of FIG. 4A). Another user input can initiate movement of the haptic device to the robotic system (e.g., indicated by the switch in the upper position of FIG. 4A).

FIG. 4B shows an example of yet another schematic illustration of a remote trauma assessment flow 335 in accordance with some embodiments of the disclosed subject matter. As shown in FIG. 4B panel (a) shows 2D and 3D scanning with the trauma assessment system, panel (b) shows region of interest identifications, panel (c) shows distances A, B, C, D, and E (e.g., which can be used to determine the FAST scan locations), panel (d) shows FAST scan locations, panel (e) shows a remote user manipulating a haptic device, panel (f) shows an image of a wound and corresponding artificial potential fields (“AFPs”), and panel (g) shows a GUI presented (or displayed) to a user.

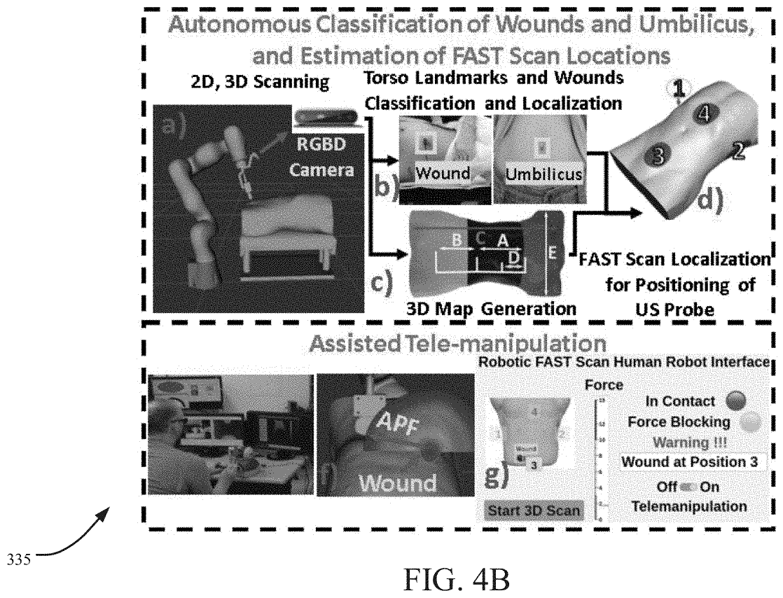

FIG. 5 shows an example of a flowchart of a process 400 for remote trauma assessment using a robotic system 100 in accordance with some embodiments of the disclosed subject matter. In some embodiments, at 402 process 400 can include generating the 3D point cloud, which can be implemented using the discussion above (e.g., at 3D point cloud 242 of FIG. 3), and on a suitable device (e.g., the computing device 106, or mobile device 110). For example, the 3D point cloud can be generated using at least two images taken from different imaging planes. At 404, process 400 can determining FAST scan locations at 404. In some embodiments, the 3D point cloud can be projected into different planes to determine different dimensions of the 3D point cloud (and thus the patient). The calculated dimensions can be compared to an atlas (e.g., a CT imaging scan of the patient), and an anatomical reference location (e.g., the umbilicus) to determine the specific coordinates of the FAST scan locations. Additionally or alternatively, in some embodiments, the FAST scan locations can be identified or confirmed by the user (radiologist) selecting points (or a group of points) on the 3D point cloud, and/or a representation of the 3D point cloud. In a more particular example, the user can select (e.g., via a mouse, the haptic device, etc.), and the computing device 106, and/or using the inputs 148 and the mobile platform 110, to determine the FAST scan locations, and subsequently store them within the computing device 106, and/or the mobile platform 110.

At 406, process 400 can include positioning the ultrasound probe 118 to a specific FAST scan location. For example, the user can select (using the inputs 128 and the computing device 106) one of the FAST scan locations that have been previously identified (e.g., at 404). As detailed above, the FAST scan location can correspond to a coordinate (or coordinate range) that when instructed to the robotic system 114, causes the ultrasound probe 118 to move to that particular location (or a location range). In some embodiments, the ultrasound probe 118 can be placed at the particular location, the user can select (using the inputs 128 and the computing device 106) or enable usage of the haptic device 108 to transmit movement instructions to the robotic system 114. For example, the user (radiologist) can manipulate the stylus of the haptic device 108 until the ultrasound probe 118 is positioned at a desirable location (e.g., at 408). In some embodiments, or while moving the haptic device 108, the user can receive ultrasound images (captured by the ultrasound probe and transmitted to the computing device 106, via the mobile device 110). Similarly, while moving the haptic device 108 (or while the user selection can be enabled) the user can view presented images (e.g., from the ultrasound probe 118, and cameras 112, 116) and the (normal) force values (from the force sensor 120), where the images and force sensor values can be transmitted to the computing device 106, via the mobile device 110 and displayed accordingly. In some embodiments, the information received by the computing device 106 can be displayed (e.g., via the display 126) to the user (radiologist). In some embodiments, the displayed information may allow the user to effectively adjust the orientation (or position) of the haptic device 108 to move the ultrasound probe 118. This way, the tactile proficiency of the user can be telecommunicated to the mobile device 110, while the user can be remote to the subject (e.g., the trauma patient).

At 410, process 400 can determine whether the user has finished acquiring ultrasound images at the specific FAST scan location. For example, if process 400 determines that no user input has been received (within a time period) to move to a new FAST scan location, the process can return to 408 and continue receiving input from a remote user to control the ultrasound probe. Alternatively, if the user has finished imaging at 410, the process 400 can proceed to determining whether additional FAST locations can be desired to be imaged at 412. For example, the user, via the inputs 128, can select a user input, and the computing device 106 can proceed to 412 after receiving the user input. Alternatively, if at 410 the process 400 determines that imaging has not been completed (e.g., such as by a lack of a received user input) the process 400 can proceed back to receiving input from a remote user at 408. In some embodiments, if at 412, the process 400 determines that additional FAST scan locations are desired to be imaged, such as by receiving a user input for the additional FAST scan location, the process 400 can proceed back to position the probe at a FAST scan location at 406. Alternatively, if at 410 additional FAST scan locations are not desired (e.g., such as with a user input or lack thereof during a time period, such as after 410) the process 400 can proceed to 414 to annotate, store, and/or transmit acquired images. Upon receiving sufficient ultrasound images at specific FAST locations, the user (radiologist) can store the images (e.g., within the computing device 110), can annotate images, such as, highlighting portions of the image indicative of a disease state (e.g., hemorrhage), and can transmit ultrasound images (including highlighted images) using the mobile platform 110.

FIG. 6 shows an example of a flowchart of a process 500 for automatically determining FAST scan locations for a robotic system in accordance with some embodiments of the disclosed subject matter. In some embodiments, process 500 can be implemented using the computing device 106, the mobile platform 110, or combinations thereof, in accordance with the present disclosure. At 502 the process 500 can include acquiring 3D imaging data, such as at 240 of flow 234. At 504, process 500 can include generating a 3D point cloud from the 3D imaging data, such as at 242 of flow 234. At 506, process 500 can include determining the width, height, and length of the subject from the 3D point cloud. For example, in some embodiments, the 3D point cloud can be projected onto different planes to determine the length, width, and height of the 3D point cloud (e.g., the subject), such as with reference to 250 of process 234. At 508 process 500 can include determining reference locations using the 2D images and the atlas. For example, the 2D images, acquired from the fixed camera 112, can be inputted into a machine learning model, which can label, and extract regions of interest within the inputted image. In some embodiments, the regions of interest can be identified by category, and spatial location within the image. In some embodiments, the regions of interest the image can be registered to the 3D point cloud to determine the coordinates and category (e.g., a bandage) of the region of interest on the 3D point cloud. At 510 process 500 can include determining FAST scan locations, such as described above at 248 of flow 234. For example, the atlas (which can be a CT 3D model having FAST scan locations) can be used along with the extracted location to determine the fast scan locations at 510. At 512, process 500 includes mapping the FAST scan locations to the 3D point cloud.

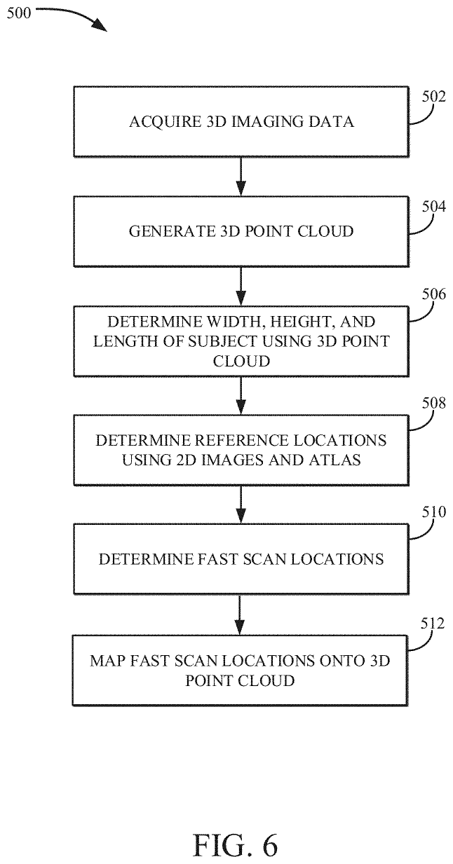

FIG. 7 shows an example of FAST scan locations plotted on a 3D point cloud 516 using an atlas 514, in accordance with some embodiments of the disclosed subject matter. The atlas 514 is shown having known atlas FAST scan locations, illustrated as 1, 2, 3, and 4. Additionally, the atlas 514 has a known length, width, and height. As described above, the 3D coordinate for one of the FAST scan coordinates on the atlas 514 (e.g., 1) can be related to the ratio of the length of the atlas to the lp (length of the subject), ratio of the width of the atlas to the wp (width of the subject), and ratio of the length of the atlas to the lp (length of the subject) to determine the coordinates of the FAST scan location relative to the 3D point cloud 516.

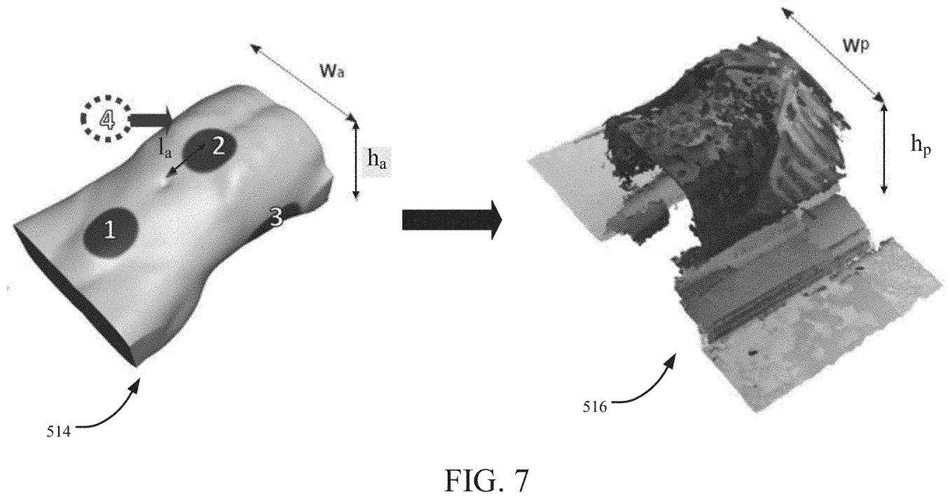

FIG. 8 shows an example of a flowchart of a process 600 for training and utilizing a machine learning model in accordance with some embodiments of the disclosed subject matter. In some embodiments, process 600 can be implemented using the computing device 106, the mobile platform 110, or combinations thereof, in accordance with the present disclosure. The process 600 can begin at 602 with training the machine learning model to classify features (e.g., wounds, umbilicus, bandages, mammary papillae, etc.). For example, in some embodiments, process 600 can further train a pre-trained general image classification CNN such as AlexNet trained on ImageNet (e.g., as described in Krizhevsky et al., “ImageNet Classification with Deep Convolution Neural Networks,” Advances in Neural Information Processing Systems 25, 2012, which is hereby incorporated by reference herein in its entirety) to classify images of wounds, bandages, and umbilici using one or more transfer learning techniques as described below in connection with Table 2. In some embodiments, process 600 can use any suitable training images of the various classes, and/or negative examples that do not correspond to any of the classes. In some embodiments, training images can be generated for various classes, such as wounds, bandages, and umbilici. The training images can also be annotated and/or segmented, and in some embodiments cross verified by a user (e.g., a radiologist) to ensure correct labeling. In some embodiments, to limit the amount of variation in the input data, wounds can be restricted to a specific wound (e.g., gunshot wounds), while bandages can be restricted to specific bandages (e.g., white bandages in abdominal region).

In some embodiments, the machine learning model trained at 602 can be a faster R-CNN. More information regarding the architecture and training of a faster R-CNN model can be found in Ren et al., “Faster R-CNN: Towards Real-Time Object Detection with Region Proposal Networks,” which is hereby incorporated by reference herein in its entirety.

At 604, process 600 can train a machine learning model to identify one or more candidate regions in an image that are likely to correspond to a class for which the machine learning model was trained at 602. For example, the machine learning model can use a trained image classification CNN (e.g., the image classification CNN trained at 602) as a feature extractor to identify features corresponding to wound, umbilicus, and/or bandage image data (e.g., among other features), which can be used in training of a region proposal network and a detection network for the identification of candidate regions, and/or regions of interest within a corresponding image.

For example, process 600 can use images depicting the classes on which the classification network was trained at 602 to train a machine learning model to identify candidate regions in the images. In such an example, the training images can be cropped such that the wound, umbilicus, and bandage (or other feature) occupies over 70 percent of the image, which can facilitate learning of the differences between the three classes.

In some embodiments, additional training images can be generated by augmented the cropped images make the classification and detection more robust. Examples of augmentation operations include random pixel translation, random pixel rotation, random hue changes, random saturation changes, image mirroring, etc.

At 606, process 600 can use the machine learning model by providing an image to the machine learning model (e.g., from fixed camera 112 and/or mobile camera 116). In some embodiments, images from fixed camera 112 and/or mobile camera 116 can be formatted to have similar characteristics to the images used for training at 602 and/or 604. For example, similar aspect ratio, similar size (in pixels), similar color scheme (e.g., RGB), etc.

In some embodiments, at 608, process 600 can receive an output from the machine learning model indicating which class(es) is present in the input image provided at 606. In some embodiments, the output can be in any suitable format. For example, the output can be received as a set of likelihood values associated with each class indicating a likelihood that each class (e.g., wound, bandage, umbilicus) is present within the image. As another example, the output can be received as a label indicating that a particular class is present with at least a threshold confidence (e.g., at least 50% confidence, at least 70% confidence, at least 95% confidence, etc.).

At 610, process 600 can receive an output from the machine learning model that is indicative of a region(s) within the input image that correspond to a particular class(es). For example, a region can be defined by a bounding box labeled as corresponding to a particular class.

In some embodiments, process 600 can carry out 608 and 610 in parallel (e.g., substantially simultaneously) or serially in any suitable order. In some embodiments, multiple trained machine learning models can be used in which the output of one machine learning model (e.g., an image classification model) can be used as input to another machine learning model (e.g., as a feature input to a region identification model).

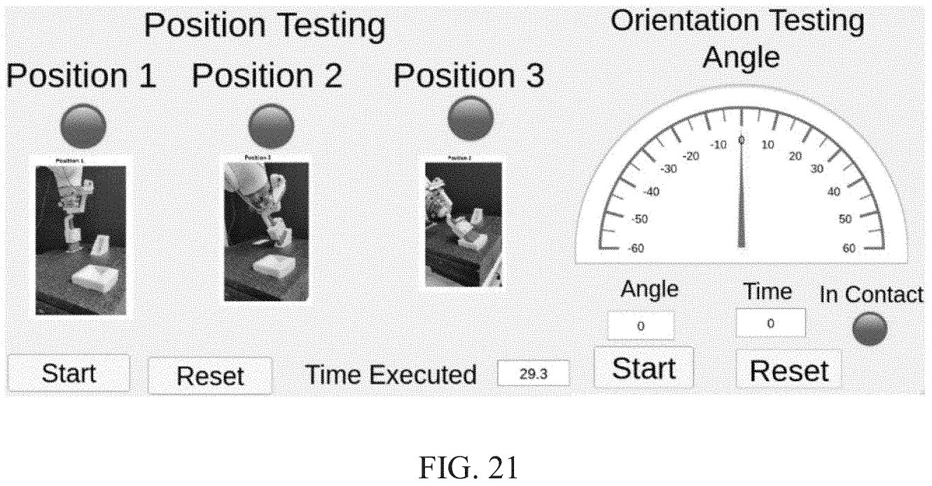

At 612, process 600 can map one or more regions of interest received at 610 to the 3D point cloud. For example, the outputted image can be registered to the 3D point cloud, and based on the output of the classification model received at 608, can be used to map a location(s) to avoid (e.g., bandages, wounds), and/or landmarks to use for other calculations (or determinations), such as the umbilicus. Note that although bandages and wounds are described as examples of objects for which the location can be mapped such that the object can be avoided, this is merely an example, and process 600 can be used in connection with any suitable object to be avoided during an ultrasound procedure, such as an object protruding from the skin of a patient (e.g., a piercing, an object which has become embedded during a traumatic injury, etc.), clothing, medical equipment (e.g., an ostomy pouch, a chronic dialysis catheter, etc.), etc. In some embodiments, at 602, the process 600 can be trained to identify such objects to be avoided. Additionally or alternatively, in some embodiments, process 600 can map regions to be avoided by positively identifying regions to that are permissible to scan, such as regions corresponding to skin. For example, process 600 can be modified (e.g., based on skin segmentation techniques described below in connection with FIG. 24) to map areas that do not correspond to skin as areas to be avoided.

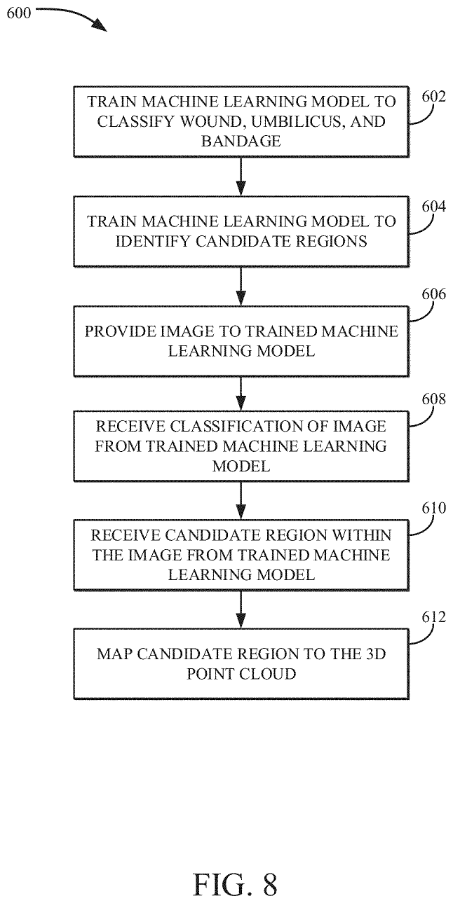

FIG. 9 shows an example of a flow for training and utilizing a machine learning model in accordance with some embodiments of the disclosed subject matter. As shown in FIG. 9, training images in panel (a) that were cropped and labeled can be provided as training input into a pre-trained general image classification CNN to act as a feature extractor for the class(es) of interest (e.g., umbilicus), and a separate group of labeled training images with bounding boxes placed around the region of interest in panel (b) can be used to train a faster R CNN, which can provide an output that has a labeled bounding box on an input image as shown in panel (c).

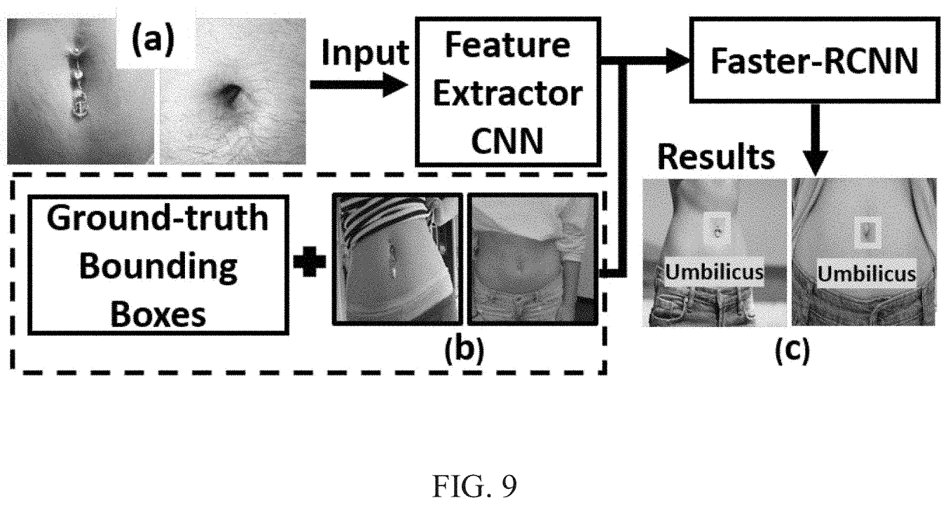

FIG. 10 shows examples of images that have been used to train a machine learning model to classify images and extract candidate regions in accordance with some embodiments of the disclosed subject matter, and images classified using a machine learning model trained in accordance with some embodiments of the disclosed subject matter. The uppermost image in the first column is a training image. The central image in the first column is the classified image, with a label 616 of a candidate region of a gunshot wound. The lowermost image in the first column is a zoomed-in image of the candidate region. The second column of images pertains to an umbilicus. The uppermost image in the second column is the training image. The central image in the second column is the classified image, with a label 618 of a candidate region of an umbilicus. The lowermost image in the second column is a zoomed-in image of the candidate region. The third column of images pertains to a bandage. The uppermost image in the third column is the training image. The central image in the third column is the classified image, with a label 620 of a candidate region of a bandage. The lowermost image in the third column is the zoomed-in image of the candidate region.

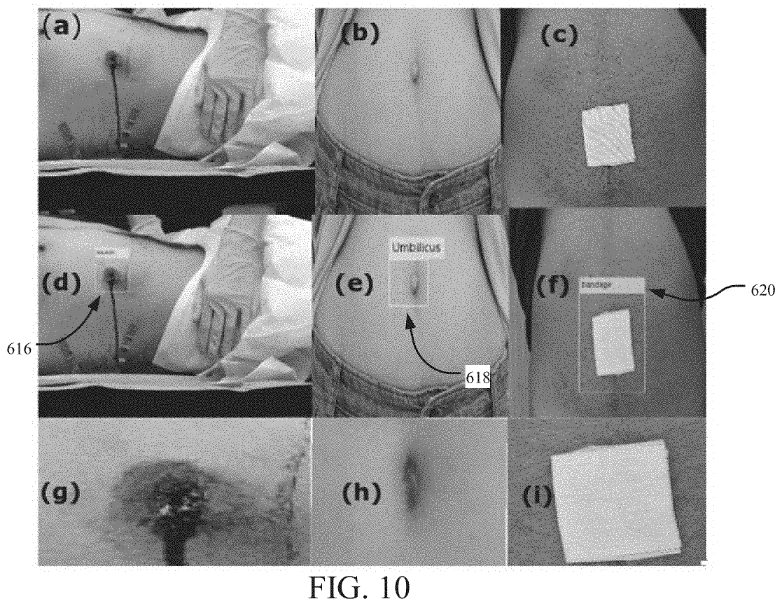

FIG. 11 shows an example of a flowchart of a process 700 for tele-manipulation of a robotic system in accordance with some embodiments of the disclosed subject matter. The process 700 can be completed using the computing device 106, the mobile platform 110, or combinations thereof, in accordance with the present disclosure.

At 702, process 700 can begin remote feedback control when a robot arm (e.g., robot arm 122) moves an ultrasound probe (e.g., ultrasound probe 118) to a first predetermined location (e.g., first FAST scan location). In some embodiments, in response to the ultrasound probe (e.g., the ultrasound probe 118) moving to a particular location, process 700 can transmit an instruction (e.g., to the computing device 106) to enable toggling of a graphical user interface switch (e.g., on a display 126). In some embodiments, prior to enabling toggling of a GUI switch, the GUI switch can be disabled (e.g., greyed out). In some embodiments, the computing device 106 can receive a toggling of the user interface switch from the user, which can cause the computing device 106 to request at least partial control of the robotic system 114.

At 704, process 700 can receive user input (e.g., via the computing device 106 and/or haptic device 108) and can include receiving movement information corresponding to movements of the haptic device 108. In some embodiments, process 700 can transmit the movement information directly (e.g., directly to the mobile platform 110, robot system 114, and/or robot arm 122). Additionally or alternatively, in some embodiments, process 700 can transform the movement information (e.g., by calculating for movements of the robotic system 114 and/or robot arm 122, and/or appropriately scaling) prior to transmission (e.g., to the mobile platform 110, robot system 114, and/or robot arm 122).

At 706, process 700 can determine and/or transmitting movement information and/or commands for the robotic system 114 and/or robot arm 122, which are based on the movements of the haptic device 108. For example, as described above in connection with FIG. 3, the movement information can be transmitted as incremental movements based on movement of the haptic, and can be limited to translations or orientation changes, in some embodiments.

At 708, process 700 can present an ultrasound image, one or more images of the patient (e.g., from the fixed camera 112 and/or the mobile camera 116), and force information (e.g., based on a force value from force sensor 120). For example, process 700 can receive ultrasound images, RGB images, and/or force information from mobile platform 110, and can use the information to populate a graphical user interface displayed by display 126. In some embodiments, the displayed information may allow the user (e.g., a radiologist) to remotely adjust the position and/or orientation of the ultrasound probe 118 via the haptic device 108 based on information gathered from the patient by the mobile platform 110.

At 710, the process 700 can receive a user input for a next FAST scan location. For example, a user input may be received, via an actuatable user interface element provided via a graphical user interface. In some embodiments, at 710 if an input has not been received to move to a new location, the process 700 can proceed back to 704 to continue to receive haptic device movement information as feedback information is presented at 708.

Otherwise, if an input has been received to move to a new location, the process 700 can proceed to 712, at which process 700 can inhibit the remote feedback control, and transmitting control to the robot system 114 to move the robot arm to the next selected location. For example, if the system 100 is implemented using a toggle for activation of feedback control, the computing device 110 can initiate the toggle (or deactivate the toggle) to prevent the user from controlling the robot system 114 while the robot arm 122 is being positioned at a next FAST scan location.

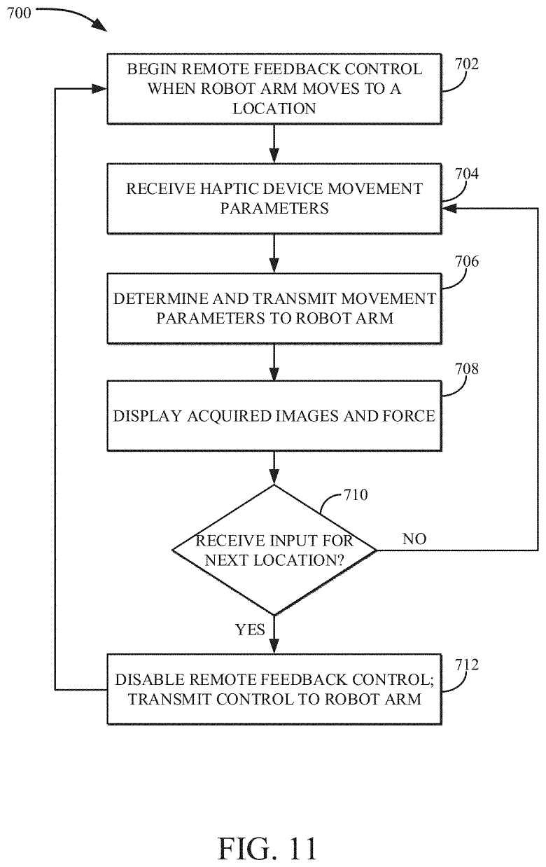

FIG. 12A shows an example of a portion of graphical user interface 714 presented by a display (e.g., the display 126) in accordance with some embodiments of the disclosed subject matter. The graphical user interface 714 can include a graph 716, a meter 718, a FAST location representation image 720, a 3D scan initiation button 722, a tele-manipulation toggle switch 724, and an ultrasound image 726. The graph 716 can be a chart which can plot the force from the force sensor 120. In some embodiments, the graph 716 displays “tool not in contact,” if the force value (e.g., from the force sensor 120) is below a threshold value (e.g., three N). In some embodiments, the force value can be above the threshold value, the force graph begins displaying values. The meter 718 can display the force value as a magnitude of the normal force component (e.g., similarly to a speedometer). The FAST location representation image 720 can illustrate four actuatable locations (e.g., via a selection of the specific location). When a user actuates one of the four locations, the computing device 110 can transmit an instruction to the mobile platform 110, which can instruct the robotic system 114 to move the ultrasound probe 118 to the selected location (e.g., one of the four locations), as described above, for example, in connection with process 500 of FIG. 6. Although the four locations can be superimposed over an image of a torso, in other embodiments, in some embodiments, the torso image can be omitted. Alternatively, the four locations can be superimposed over an image and/or 3D model of the patient (e.g., based on an image acquired by fixed camera 112 and/or mobile camera 116).

The 3D scan initiation button 722, when actuated by a user, causes the computing device 106 to transmit an instruction to the mobile platform 110, which can cause the robotic system 114 to acquire 3D imaging data (e.g., to initiate 502 of process 500). The tele-manipulation toggle switch 724, when actuated, causes the mobile platform 110 to transmit movements received by the haptic device 108 into movements of the ultrasound probe 118 (e.g., initiating remote control of the robotic system 114). When the toggle switch 724 is deactivated, the robotic system 114 does not instruct (or receive) the movements transmitted by the mobile platform 110. The ultrasound image 726 can be a real-time ultrasound image, acquired by the ultrasound probe 118, transmitted to the computing device 106 from the mobile platform 110. In some embodiments, when the ultrasound imaging data (or image) is received by the computing device 106, the computing device 106 displays the ultrasound image 726 on the display 126.

FIG. 12B shows an example of another portion of a graphical user interface 714 presented that can be presented by a display (e.g., 126) in accordance with some embodiments of the disclosed subject matter. In some embodiments, the graphical user interface 714 can include an image of the field of view 728, and an image outputted from a machine learning model 730 (e.g., the machine learning model 244). The image of the field of view 728 can be acquired from the fixed camera 112. The image outputted from the machine learning model 730 can be an image that was inputted into a machine learning model to classify the image, and extract (and/or identify) candidate regions. As shown in FIG. 12B, the machine learning model 730 has identified the candidate region 732 and has classified the portion of image within the candidate region 732 as including an umbilicus. Although FIGS. 12A and 12B have suggested that the graphical user interface 714 be displayed on a single display 126, in some embodiments, portions of the graphical user interface 714 can be displayed on multiple displays.

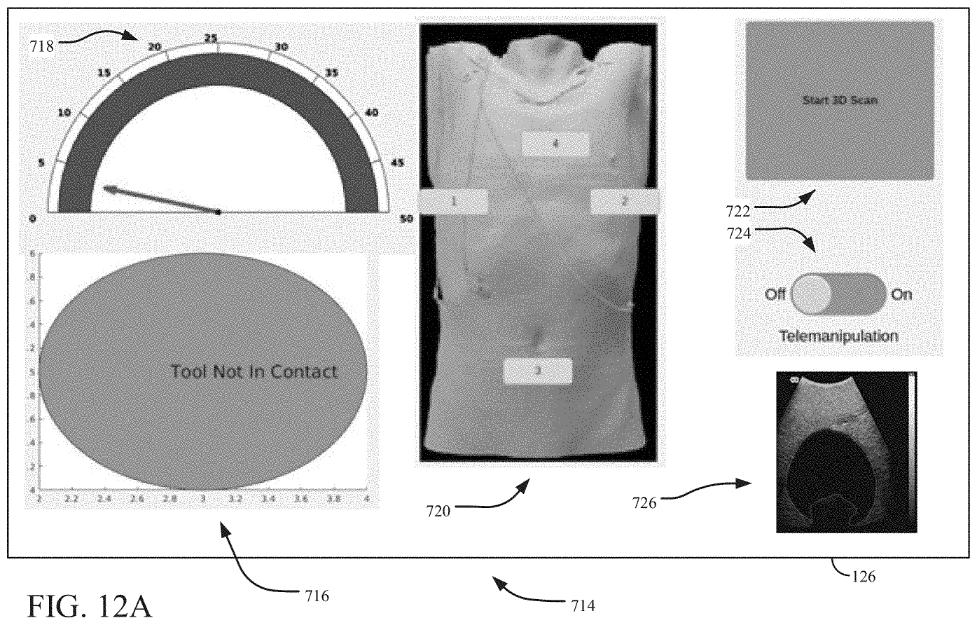

FIG. 13A shows an example of a remote portion of a trauma assessment system 800 implemented in accordance with some embodiments of the disclosed subject matter. The trauma assessment system 800 can include a robot 802, a tool camera 804 (similar to the mobile camera 116), a force sensor 806, an ultrasound probe 808, and a fixed camera (an RGB camera not shown). FIG. 13A also shows an ultrasound phantom 810, which is the FAST/Acute Abdomen Phantom from Kyoto Kagaku (Kyoto Kagaku Co. Ltd, Kyoto, Japan). The phantom 810 has realistic features, such as internal hemorrhages at all four FAST locations including at the pericardium and bilateral chambers as well as intra-abdominal hemorrhages around the liver, the spleen, and the urinary bladder.

FIG. 13B shows an example of the remote portion of a trauma assessment system 800, as shown in FIG. 13A, with labeled components and joints, in accordance with some embodiments of the disclosed subject matter. As shown in FIG. 13B, panel (a) shows the robot and the relative placement of a force sensor, mobile camera, and ultrasound probe with relation to the robot arm, panel (b) shows the three most distal joints of the robot arm which respectively control the yaw, pitch, and roll of the EE, and panel (c) shows an image acquired by the mobile camera.

FIG. 14A shows an example of a haptic device with a labeled coordinate system, in accordance with some embodiments of the disclosed subject matter. As shown in FIG. 14A, the origin can be defined at the tip of the stylus of the haptic device, with axes x_{H}, y_{H}, and z_{H} emanating from the origin. In some embodiments, the origin can be further defined by the mean (or zeroed) locations of each of the joints of the haptic device. As also shown in FIG. 14A, the joint which couples the haptic device to the stylus can be defined as the reference point for translation movement.

FIG. 14B shows an example of the haptic device of FIG. 14A, with joints J4, J5, J6 and corresponding rotational terms, in accordance with some embodiments of the disclosed subject matter. As shown in FIG. 14A, joints J4, J5, and J6 are the most distal joints of the haptic device, and respectively control the roll, yaw, and pitch of the haptic device.

FIG. 15 shows an example of coordinate systems for the haptic device, for the mobile camera, and for the robot arm, in accordance with some embodiments of the disclosed subject matter. As shown in FIG. 15, the panel (a) shows a coordinate system of a haptic device, panel (b) shows a coordinate system of a mobile camera superimposed on an image acquired by the mobile camera, and panel (c) shows the end effector coordinate system of the robot. Note that the coordinate systems shown in FIG. 15 are congruent with each other such that a translation of the stylus of the haptic device in the +zH direction causes a corresponding movement of the robot EE in the +zR direction, which can be observed in the tool view as a decrease in size of objects in the field of view as the mobile camera moves away from the scene axially in the +zC direction (e.g., out of FIG. 15 toward the viewer).

The robotic software architecture (and graphical user interface) was developed to control the remote trauma assessment system, which included a control system having planning algorithms, robot controllers, computer vision, and the control allocation strategies being integrated via Robot Operating System (“ROS”). More information regarding the ROS system can be found in Quigly, et al., “ROS: an open-source robot operating system,” which is hereby incorporated by reference herein in its entirety. Smooth time-based trajectories were produced between the waypoints using Reflexxes Motion Libraries. More information regarding the Motion libraries can be found at, “Reflexxes motion libraries for online trajectory generation,” (available at reflexes.ws), which is hereby incorporated herein in its entirety. A Kinematics and Dynamics Library (“KDL”) in Open Robot Control Systems (“OROCOS”) was used to transform the task-space trajectories of the robot to the joint space trajectories, which is the final output of the high-level autonomous control. More information regarding the library can be found at Smits, “KDL: Kinematics and Dynamics Library,” (e.g., available at orocos(dot)org/kdl), which is hereby incorporated herein in its entirety. Finally, IIWA stack helps to apply the low-level controllers of the robot to follow the desired joint-space trajectories. More information regarding the IIWA stack can be found in Hennersperger, et al., “Towards MM-based autonomous US acquisitions: A first feasibility study,” which is hereby incorporated by reference herein in its entirety. A Graphical User Interface, such as graphical user interface 714, was developed in MATLAB and linked to ROS, for the user to command the robot, display instantaneous forces on the probe, initiate 3D scan, move robot to each initial scan location, and switch tele-manipulation. More information regarding the ROS system can be found at, “Robot operating system (ros) support from robotics system toolbox—matlab,” (available at www(dot)mathworks(dot)com/hardware-support/robot-operating-system), which is hereby incorporated by reference herein in its entirety. In this example, two other screens showed live feeds from world camera, tool camera, and the US system.

In some embodiments, the reference frames between tool (ultrasound probe), camera, and haptic device can be determined. The robot is tele-operated in the tool frame which is defined at the tip of the ultrasound probe. This allowed the operator to reorient the probe without any change in the position of the probe tip. This enabled the sweeping scanning motions necessary for FAST, while the probe tip maintained the position in contact with the phantom. Even though the operator perceived the scene in the camera frame through the GUI, it was observed that the fixed relative transformation between the camera and tool frames does not prevent the user from operating intuitively. The operator uses visual feedback from the RGB cameras to command the robot using a Geomagic Touch haptic device. In this example, the haptic device has 6 DOFs, one less than the manipulator, and a smaller workspace. For tele-manipulation, the robot arm is autonomously driven to each initial FAST location.

The machine learning model (e.g., the machine learning model 244) was implemented as a Faster R-CNN. The Faster R-CNN model was trained from a pre-trained network using transfer learning techniques. AlexNet trained on ImageNet was imported using Matlab's Machine Learning Toolbox and the fully connected layer of AlexNet was modified to account for 3 classes (wounds, umbilicus and bandage). After training the classifications, the Faster R-CNN model was trained on the cropped augmented images. Using stochastic gradient descent (“SGDM”) and an initial learning rate of 1e-4 the network converged in 10 epochs and 2450 iterations. For the faster R-CNN model, the model was trained on 858 wound images, 2982 umbilicus images, and 840 bandage images. Some of the wound images were taken from Medetec Wound Database (e.g., available at www(dot)medetec(dot)co(dot)uk/files/medetec-image-databases) and the rest of the images for wound, umbilicus and bandage were obtained from Google Images.

The CNN trained previously was used as a feature extractor to simultaneously train the region proposal network and the detection network. The positive overlap range was set to [0.6 1], such that if the intersection over union (“IOU”) of the bounding box is greater than 0.6, it will consider it as a positive training sample. Similarly the negative overlap range was [0 0.3]. Using SGDM, the network finished training the region proposed network and the detection network in 1,232,000 iterations. Before calculating the final detection result some optimization techniques on the bounding box (“BB”) were also applied. This included removing any BB with aspect ratio less than 0.7, and the width and height of BB were constrained to 300 pixels. These threshold values were experimentally determined. Additionally, any BB out of the skin is removed. The skin detection was carried out by color thresholding in RGB, YCrCb and HSV color spaces.

Generally, to train the feature extractor cropped image data was augmented to increase training data and make classification and detection more robust. A total of 604 original wound images, 350 umbilicus images, and 200 bandage images were used, which after the data augmentation amounted to 3539, 2100, and 1380 images, respectively. In some embodiments, the data was split randomly into 70:30 percentage of training data and validation data. A separate data set for testing was also created, which consisted of 143 wound, 382 umbilicus and 140 bandage images.

For some uses, faster R-CNN has advantages over other machine learning models. For example, the Faster R-CNN for object detection can simultaneously train the region proposal network and the classification network, and the detection time can also be significantly faster than other region based convolution neural networks. The simultaneous nature of the Faster R-CNN can improve the region proposal quality and estimation as opposed to using fixed region proposals, such as in selective search.

The phantom (e.g., the phantom 810) was scanned with the robotic system and four FAST scan positions were estimated using atlas based scaling as described previously. Table I (below) shows estimated positions and actual positions of the four FAST scan. Actual positions can be the positions where the FAST scan locations can be on the phantom, manually selected by an expert radiologist. The average position accuracy of the system was 10.63 cm±3.2 cm.

TABLE 1
estimated and actual FAST scan positions

| | Estimated | Actual | Accuracy |
|---|---|---|---|
| | positions (m) | positions (m) | (m) |
| Pt 1 | (0.572, 0.143, 0.333) | (0.603, 0.160, 0.239) | 0.100 |
| Pt 2 | (0.579, −0.220, 0.333) | (0.547, −0.130, 0.236) | 0.136 |
| Pt 3 | (0.367, −0.037, 0.333) | (0.363, 0.017, 0.299) | 0.063 |
| Pt 4 | (0.580, −0.040, 0.333) | (0.699, −0.010, 0.310) | 0.124 |

The feature extractor CNN used sensitivity (recall or true positive rate) and specificity (True Negative rate) as metrics, as shown in Table 2 (below). The sensitivity and specificity for all the three classes can be inferred to be above 94 percent and therefore can be used as a base network for the Faster R-CNN model. In one example, the overall accuracy for the classifier is 97.9 percent. The increased sensitivity and specificity for the umbilicus class can be explained by the significant higher amount of training data as compared to the other classes. In some embodiments the Faster R-CNN model was trained, the Mean Average Precision (“mAP”) was calculated on the test data (e.g., Table 2) at an Intersection Over Union (“IOU”) of 0.5. This metric was followed as per the guidelines in PASCAL VOC 2007. The mAP for the three classes at an IOU of 0.5 was 0.51, 0.55, and 0.66 respectively for umbilicus, bandage, and wounds as shown in FIG. 16A, while the precision vs. recall values for the three classes can be seen in FIG. 16B.

FIG. 16A shows a graph of mean average precision vs. intersection over union (“IoU”) for images of bandages, umbilici, and wounds in accordance with some embodiments of the disclosed subject matter. The variation in the mAP with respect to IOU is shown in FIG. 16A.

FIG. 16B shows a graph of precision vs. recall with IoU held constant at 0.5 in accordance with some embodiments of the disclosed subject matter. FIG. 16B supports that a recall or sensitivity value of 0.7 or when 70 percent of the detection can be correctly made, the wound class is correctly classified 76 percent of the time, the umbilicus class 68 percent of the time, and bandage class 78 percent of the time.

TABLE 2
evaluation of results for Feature Extractor CNN

| | Sensitivity | | |
|---|---|---|---|
| Class Name | (Recall) | Specificity | Accuracy |
| Wounds | 96.08% | 94.78% | 97.9% |
| Umbilicus | 98.3% | 98.14% | |
| Bandage | 96.1% | 94.23% | |

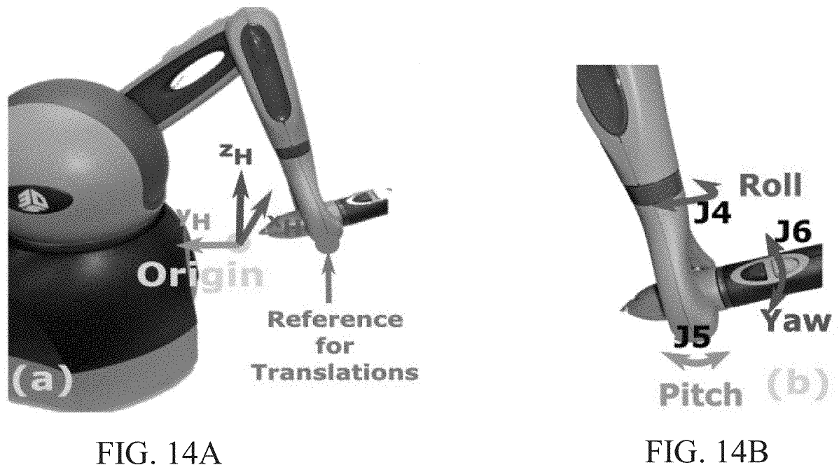

FIG. 17 shows a graph of forces recorded during a FAST scan at position three, during an example in accordance with some embodiments of the disclosed subject matter. As shown in FIG. 17, it was found that the magnitude of force acting on the scanning of the probe may increase even when the virtual fixture was active due to orientation changes of the probe. The maximum normal force recorded during the complete scan was 6.3 N at FAST scan location 2. The average normal force during scanning when the probe was in contact with the phantom was found to be 5.4 N. The hard virtual fixture (e.g., 12 N) was never activated during the actual scan as the forces never breached the threshold. The force threshold for the soft virtual fixture (e.g., as described in above with regard to FIG. 3) was set to 3N based on quality of images by trial and error.

A 34 minute long training about the robotic system, which also encompassed practice for tele-manipulation was given to the expert radiologist. Following the training, a complete FAST scan of the phantom was conducted. For the scanning procedure using the robotic system, the position of the umbilicus along with the 3D image were used to autonomously estimate the FAST scan locations and position the robot autonomously just above consecutive FAST scan locations, as described above. Following this autonomous initialization, the remotely-located radiologist commanded the robot to move to each scan location one by one and perform a tele-manipulated FAST exam. While the radiologist manipulated the haptic device, tele-manipulation commands were transmitted to the robot. Additionally, the force value from the force sensor was transmitted to the haptic device, to provide feedback for the radiologist. In a more particular example, the force value provided to the haptic device, resisted movement by providing a force normal to the stylus of the haptic device, which was proportional to the forces acting on the ultrasound probe.

The scanning procedure using the robotic system was completed in 16 minutes and 3 seconds as compared to freehand manual scanning, which was completed in 4 minutes and 13 seconds. However, the real-time images obtained by the robot were found to be more stable and having better contrast. This can be attributed to the robot's ability to hold the probe stationary in position.

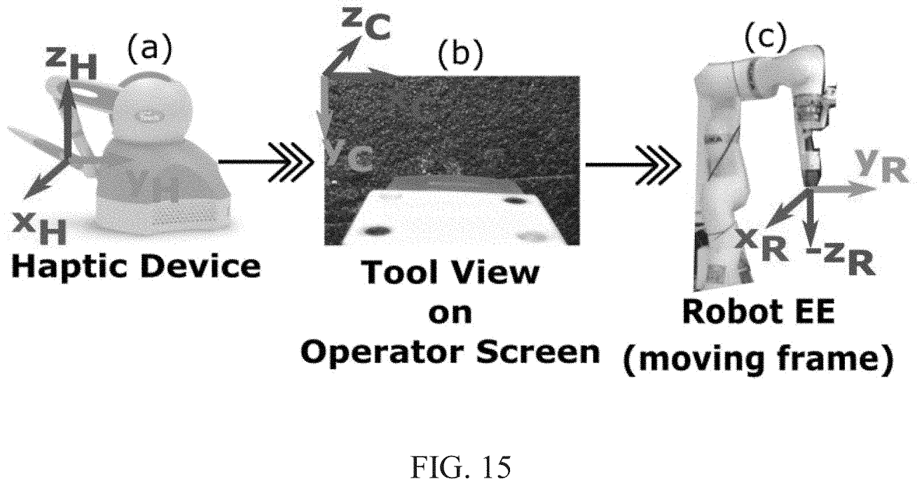

FIG. 18 shows an example of a series of ultrasound scans acquired with a remote trauma assessment system implemented in accordance with some embodiments of the disclosed subject matter and acquired manually by an expert radiologist. In FIG. 18, the pairs of scans on the left were acquired using the remote trauma assessment system, and the pairs of scans on the right were acquired by an expert radiologist using a handheld ultrasound probe.

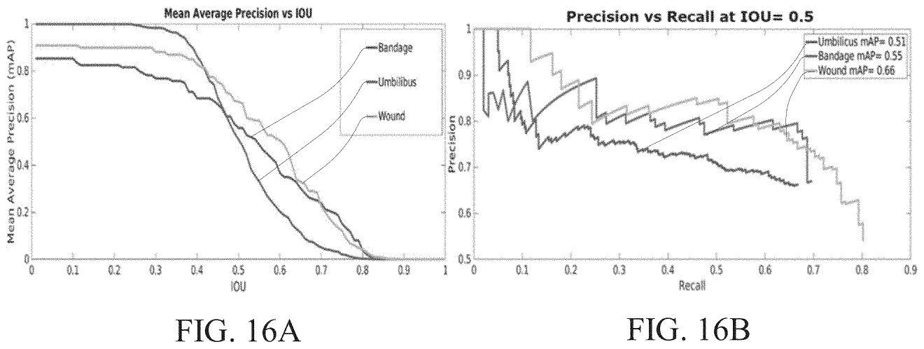

FIG. 19 shows a series of graded ultrasound scans with images in the top row corresponding to ultrasound scans acquired with a remote trauma assessment system implemented in accordance with some embodiments of the disclosed subject matter and ultrasound images in the bottom row corresponding to ultrasound scans acquired manually by an expert radiologist. A blinded radiologist graded the nine ultrasound images acquired using the remote trauma assessment system and acquired by manual scan, including the eight images shown in FIG. 19, which were chosen for best displaying the extent of the hemorrhages in the FAST phantom. The images, which were randomized to avoid any bias, were graded on a Likert scale (1-low to 5-high). The images from the robotic system received a score of 44 of possible 45 as compared to a 41 for the images from the free hand scan. Standard deviations of 0.33 and 0.53 were obtained for the robotic system and hand scan images, respectively. The two-tailed p-value for the scores was found to be 0.1284, which shows that the difference in the scores requires more data to be statistically significant. Nonetheless, the results show that the ultrasound images produced using the remote trauma assessment system are at least on par with those obtained by an expert radiologist manually. Representative images of regions of interest from each of the four FAST points along with scores received (marked in the upper left corner of each image) are shown in FIG. 19.

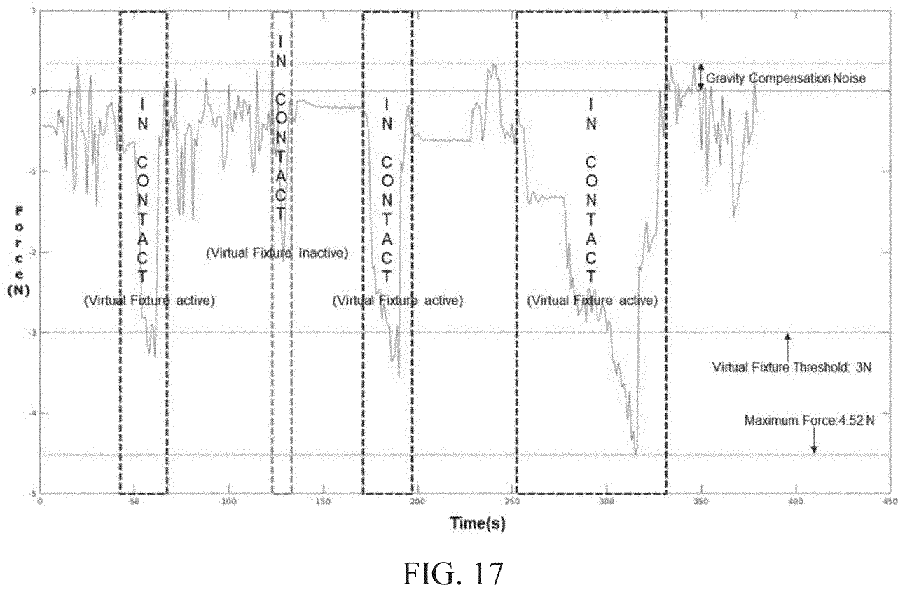

FIG. 20 shows an example of a positioning test procedure used to assess a remote trauma assessment system implemented in accordance with some embodiments of the disclosed subject matter. The procedure of FIG. 20 was used to assess the tele-manipulated FAST scan, which requires semi-accurate positioning of the probe at the scan location, followed by sweeping motions in the probe tip's roll, pitch, and yaw axes during the scan to search for organs, free fluid or other objects of interest in the intraperitoneal or pericardial spaces. In order to analyze each tele-manipulation system's performance, the procedure was divided into two sub-tasks; positioning and sweeping. The system was tested on a variety of metrics described below using human-subject trials involving 4 participants trained to operate the remote trauma assessment system. A prior tutorial was given to each participant along with a hands-on practice session before conducting the final tests. Each participant performed the positioning test followed by the sweeping test. The order of test conditions for each, positioning and sweeping test, was randomized to eliminate the effects of learning or operator fatigue.

The positioning test included a custom test rig with 3 negative 3D printed molds of the probe enlarged by 10% (see FIG. 20 panel (a)). These negative molds are inclined at 45°, 0°, and 40° as shown in FIG. 20 to simulate different FAST scan positions. During the test, the ultrasound probe must be completely inserted into the mold in the correct orientation to press a button (see FIG. 20, panels (b) to (d)) indicate a successful positioning. The test was performed using both a hybrid (Hyb) control strategy and a position (Pos) control strategy in the absence of a VF.

The sweeping test involved rotating the probe about the EE's roll, pitch, and yaw axes, individually from −30 to +30 degrees while being in contact with the foam test rig at a marked location (see FIG. 20). The test was performed using the Hyb and Pos control strategies with and without an active VF. To analyze the VF, the results using the Hyb control strategy were used to compare the forces exerted by the probe in presence and absence of the VF.

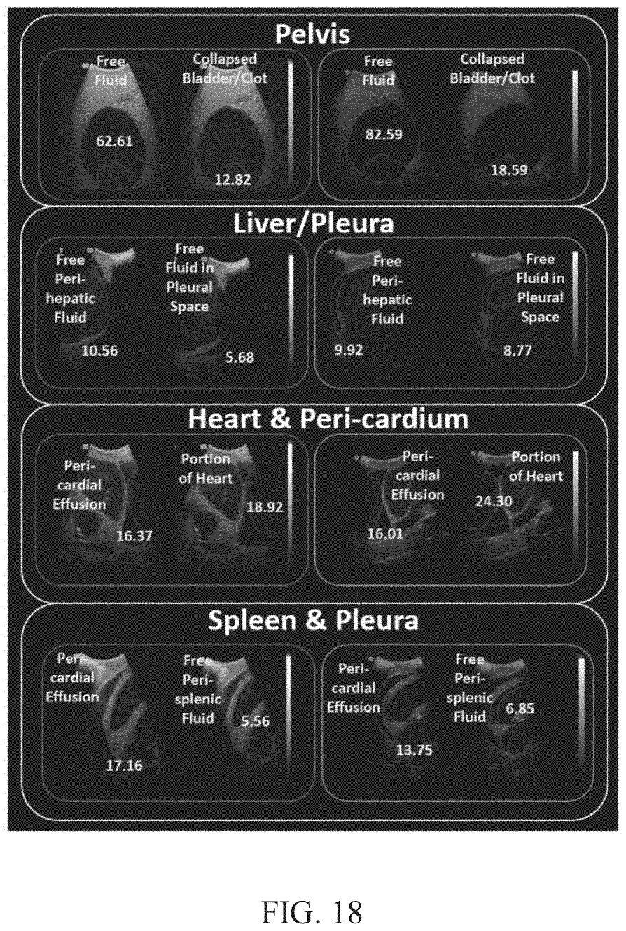

FIG. 21 shows an example of a graphical user interface implemented in accordance with some embodiments of the disclosed subject matter. The real-time status of the operator's progress during the experiments described above in connection with FIG. 20 was displayed using the graphical user interface of FIG. 21. The roll, pitch, and yaw angles for the sweeping test are shown in FIG. 21 using gauges, while the operator's progress in the positioning test is shown using colored lamps and a timer.

Performance of a remote trauma assessment system implemented in accordance with some embodiments of the disclosed subject matter was analyzed based on two main criteria: the ability of the probe to reach the desired positions in the desired orientations (positioning test) and the ability of the probe to sweep at the desired position (sweeping test). The metric for assessing the advantages of the VF was based on the consistency of forces and the maximum force exerted during the scan. The positioning test was analyzed based on the completion time and number of collisions. The former was the time taken for the operator to correctly orient and place the probe into each mold, while the latter is the number of times the probe collided with surfaces on the test-bed before fitting inside the mold. In general, the less time taken and the fewer the number of collisions, the better.

Two metrics were used to assess the quality of the ultrasound scan for the sweeping test: the velocity of sweep in each axis, and the smoothness of the sweep. The velocity was the average velocity during sweep in each axis. The faster the sweep, the better, as this allows for faster maneuverability of the probe. Smoothness, on the other hand, is measured by the standard deviation in angular velocities of each axis during the sweep. The lower the standard deviation, the smoother the sweep.

Two metrics are used to study the benefits of the VF: consistency of forces and maximum force exerted during a scan. Consistency is determined using the standard deviation of the forces along with the percentage of time during which the probe was in contact with the foam during the sweep. The higher the percentage and lower the standard deviation, the better the system's performance. The VF is responsible for maintaining a limit on the maximum forces exerted on the patient. Hence, the closer the maximum force to the VF threshold, the better.

The results for the positioning test can be seen in Table 3 below. Columns 2 and 3 of Table 3 show the total number of collisions for the positions described in FIG. 20 for each user while Columns 4 and 5 show the average time (in seconds) it took each user to complete the task for the three positions using 1) Position (Pos) and 2) Hybrid (Hyb) control strategies. The training to the users was balanced to eliminate an unfair advantage to either of the control strategies. The order in which each subject was trained can be seen in the last column of Table 3 such that “1-2” indicates that the subject performed the test using Position before hybrid control.

TABLE 3
positioning test results

| | Collisions | Time (s) | | | |
|---|---|---|---|---|---|
| Subject | Pos | Hyb | Pos | Hyb | Order |
| 1 | 0 | 1 | 90.33 | 216 | 2-1 |
| 2 | 2 | 2 | 211 | 74.33 | 1-2 |
| 3 | 9 | 2 | 101 | 43.33 | 1-2 |
| 4 | 7 | 4 | 98.67 | 67.67 | 2-1 |

As shown in Table 3, there were a total of 18 collisions using the Position control strategy as compared to 9 collisions using the Hybrid control strategy amongst the 4 participants. The average time of completion for all three positions was 125.25 seconds for Position control and 100.33 seconds for Hybrid control. Even though users perform 25% faster with Hybrid control strategy, participants performed significantly better in the test which they performed second.

For the sweeping test, the Position and Hybrid control strategies were tested using with and without an active VF totaling 4 test combinations. The results of the sweeping tests are shown in Table 4 and Table 5. Columns 2 and 4 in Tables 4 and 5 show the average angular velocities of all test subjects in each axis (in degrees/sec) while Columns 3 and 5 show the standard deviation of the group.

TABLE 4
sweeping test results without VF

| | Position | | Hybrid | |
|---|---|---|---|---|
| | Mean | SD | Mean | SD |
| Axis | (deg/s) | (deg/s) | (deg/s) | (deg/s) |
| Roll | 4.04 | 4.15 | 2.09 | 1.75 |
| Pitch | 4.10 | 3.99 | 3.89 | 2.99 |
| Yaw | 9.58 | 9.18 | 3.82 | 2.65 |
| Mean | 5.91 | 5.77 | 3.27 | 2.46 |

TABLE 5
sweeping test results with VF

| | Position | | Hybrid | |
|---|---|---|---|---|
| | Mean | SD | Mean | SD |
| Axis | (deg/s) | (deg/s) | (deg/s) | (deg/s) |
| Roll | 4.74 | 7.26 | 1.79 | 1.38 |
| Pitch | 5.01 | 4.95 | 3.04 | 2.55 |
| Yaw | 8.78 | 7.90 | 3.80 | 2.23 |
| Mean | 6.18 | 6.71 | 2.88 | 2.05 |

While comparing position and hybrid control strategies without VF in Table 4, the standard deviation of angular velocities is 58% lower for the Hybrid strategy. For the same test with VF, the standard deviation of angular velocities is 70% lower in case of hybrid strategy. Therefore, the Hybrid strategy may allow the operator to perform the ultrasound scan while scanning the probe with much more consistent angular velocities. The Position control strategy however provides 80% faster angular velocities without VF and 114% faster angular velocities with VF. Hence, the position control strategy may allow for faster reorientation of the probe. In some cases, it may be preferable to prioritize the quality of the ultrasound image over speed, as the speed of scan is insignificant as compared to the total time the patient generally spends en-route to the hospital.

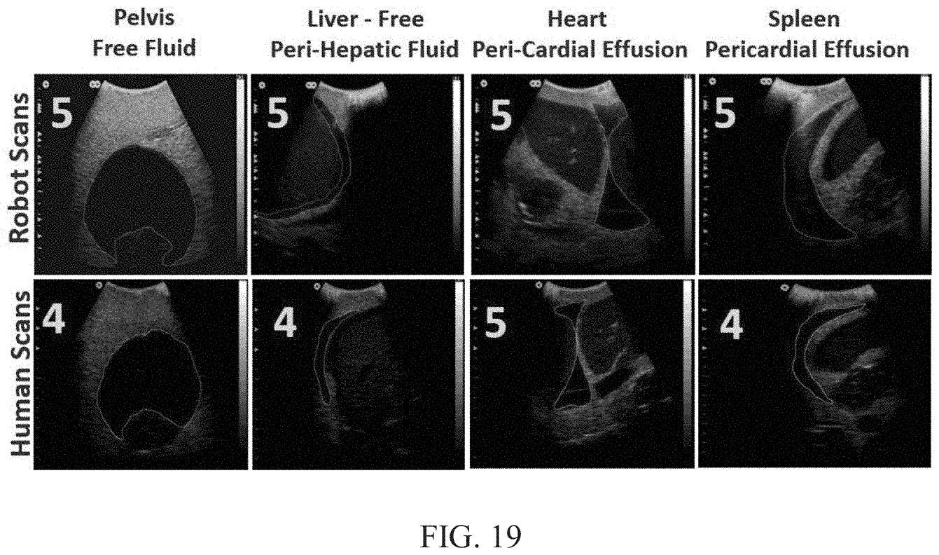

FIG. 22 shows an example of a graph of pitch angles with respect to time for one test while using a VF in a remote trauma assessment system implemented in accordance with some embodiments of the disclosed subject matter. The performance of the virtual fixture was analyzed using the Hybrid control strategy described above. The results of the sweep without and with the VF are shown in Tables 6 and 7, respectively. In Tables 6 and 7, Column 2 shows the average percentage duration of contact for the three axes, Column 3 shows the mean force (N) exerted during the sweep, Column 4 shows the standard deviation of the group, and the last column shows the maximum force (N) exerted by the probe during the scan. A force cut-off threshold of 50 N was used to deactivate the tele-manipulation system for possible safety reasons, but was never breached.

TABLE 6
hybrid control strategy results without the VF

| | No VF | | | |
|---|---|---|---|---|
| | Contact | Mean | SD | Max Force |
| | (%) | (N) | (N) | (N) |
| 1 | 40.2 | 2.3 | 3.1 | 26 |
| 2 | 44.4 | 5.2 | 7.2 | 28 |
| 3 | 44.44 | 7.26 | 11.3 | 43.3 |
| 4 | 31.3 | 6 | 10.1 | 37.3 |
| Mean | 40.085 | 5.19 | 7.925 | 33.65 |

TABLE 7
hybrid control strategy results with the VF

| | No VF | | | |
|---|---|---|---|---|
| | Contact | Mean | SD | Max Force |
| | (%) | (N) | (N) | (N) |
| 1 | 69.2 | 3.8 | 2.6 | 9.366 |
| 2 | 75.4 | 6.47 | 4.7 | 25 |
| 3 | 89 | 5.4 | 2.5 | 13.3 |
| 4 | 91.5 | 5.57 | 2.5 | 11.1 |
| Mean | 81.275 | 5.31 | 3.075 | 14.6915 |

Comparing Tables 7 and 6, it can be seen that the mean percentage duration of contact during the sweep is almost 100% higher with VF. While the average forces exerted in both cases is similar, the standard deviation is approximately 150% lower when the VF is active. The maximum force exerted by the system is also significantly closer to the desired VF threshold of 7 N when the VF is active. Despite the presence of a VF, the forces exceed the threshold because the VF only locks the probe in its current position and should not be confused with impedance control. The cause of this increase in forces is mainly due to the change in interaction forces of the probe with the test-bed.

In this example experiment, the ability of the remote trauma assessment system to accurately classify umbilicus and wounds on the classification test data was evaluated and then calculate the detection accuracy on the detection test data was calculated. The sensitivity (recall or true positive rate) and specificity (True Negative rate) of the feature extractor CNN for both the classes was above 94%. This can therefore be used as a base network to train the Faster R-CNN model. The overall accuracy for the classifier was 97.9%. Once the Faster R-CNN model was trained, the Mean Average Precision (“mAP”) was calculated on the detection test data at an IoU of 0.5. This metric was followed as per the guidelines established in PASCAL VOC 2007. The mAP for the two classes at an IOU of 0.5 is 0.51 and 0.66 for umbilicus and wounds, respectively.

In some embodiments, a remote trauma assessment system implemented in accordance with some embodiments of the disclosed subject matter can generate a warning for a radiologist if a wound was detected near a FAST exam location. Moreover, the FAST exam points are estimated with respect to the umbilicus in some embodiments. Accordingly, it can be important to accurately determine the position of these objects in the robot's frame. In an experiment described in connection with Table 8, readings were taken for objects of each of the three classes placed at 5 random positions and angles on a wound phantom to make a total of 15 tests. The ground truth values for each, umbilicus and wounds, were estimated by touching the actual center of the object using a pointed tool attached to the robot and performing forward kinematics to determine the object locations in the robot world frame.

TABLE 8
localization errors for umbilici and wounds

| Class Name | Mean Error (mm) | Standard Deviation (mm) |
|---|---|---|
| Umbilicus | 8.77 | 1.5 |
| Wounds | 9.30 | 0.79 |

The results for average error (Euclidean distance) and standard deviation for each class is shown in Table 8. The average localization error for both the classes combined was found to be 0.947 cm±0.179 cm. To evaluate the accuracy of the estimated FAST exam points, five localization phantoms were scanned with the robotic system and the four FAST scan positions were estimated using techniques described above. The actual positions are the centroids of the FAST regions on the localization phantoms (e.g., the FAST scan positions 1 and 2 are placed symmetrically on the body). Hence, their accuracies are reported together. Since the final step of the semi-autonomous FAST exam is tele-manipulated, the estimated locations need not be highly accurate and only need to be within the workspace of the haptic device and Field Of View (“FOV”) of the camera. Table 9 shows the mean error (Euclidean distance) and standard deviation between the estimated and actual positions of the four FAST exam points in the robot's base frame. The third column of Table 9 shows whether the estimated point was within this marked region. The average position accuracy of the system was 2.2 cm±1.88 cm. All the estimated points were found to be within the scanning region marked by the expert radiologist for each FAST scan location. The largest error for any location over the five test phantoms was 7.1 cm, which was well within the workspace of the slave robot and FOV of the RGB-D camera. Thus, all the FAST points were within tele-manipulatable distance from the estimated initialization positions.

TABLE 9
estimated and actual FAST scan locations

| | Mean | Standard | Inside | Inside |
|---|---|---|---|---|
| | Error (cm) | Deviation (cm) | Marked Region | Workspace |
| Pt 1, 2 | 1.66 | 1.46 | Yes | Yes |
| Pt 3 | 1.64 | 0.87 | Yes | Yes |
| Pt 4 | 3.34 | 3.31 | Yes | Yes |

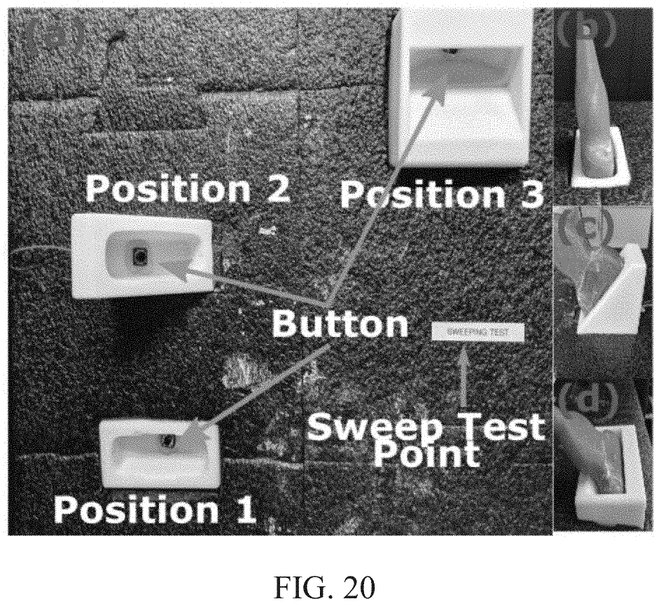

FIG. 23 shows examples of heat maps of forces and trajectories during tele-manipulation of a robotic system implemented in accordance with some embodiments of the disclosed subject matter using artificial potential fields. As shown in FIG. 23, panel (a) shows an example of a heat map of forces and trajectories during tele-manipulation of a robotic system using artificial potential filed, while panel (b) shows an example of a heat map of forces and trajectories during tele-manipulation of a robotic system without using artificial potential fields.

In this experiment, subjects were asked to tele-manipulate the robot from the start to the end point while trying to avoid a wound of 4 cm radius in the path. The resulting trajectories for 4 different subjects from a pilot study involving 8 human subjects are shown in FIG. 23. The heat map defines the amount of resultant force being exerted by the APF. From the 3 colored trajectories (with APF), it can be seen that the APF was able to guide the probe around the hemisphere and successfully avoid the wound. The trajectory in black (without APF) was able to avoid the wound but still allowed the subject to maneuver the probe very close to it. Overall, the subjects maintained an average distance of 5.09 cm in the presence of the APFs with a minimum distance of 4.575 cm to the center of the wound. However, in the absence of the APFs, the subjects maintained an average distance of 3.3 cm with a minimum distance of 1.8 cm to the center of the wound, and caused one collision with the wound. From a t-tail test, the p-value for the two groups was found to be 0.0117 proving that the data is statistically conclusive.

FIG. 24 shows an example of a process 800 for automatically labeling portions of an image corresponding to human skin in accordance with some embodiment of the disclosed subject matter. In accordance with some embodiments, the process 800 can be used in connection with mechanisms described herein for remote trauma assessment. For example, the process 800 can be used to segment skin in one or more images generated by fixed camera 112 and/or mobile camera 116, and skin segmentation can be used in connection with labeling portions of the image. For example, portions of an image not corresponding to skin can be labeled as areas to avoid (e.g., ultrasound images can only be recorded when there is exposed skin, and areas corresponding to wounds, bandages, etc., are unlikely to be labeled as skin by the process 800). However, the process 800 can be used in other contexts where segmenting skin in an image is useful, such as in gesture recognition tasks, face tracking, head pose estimation, dermatology applications, etc.

In some embodiments, at 802, the process 800 can train an automated skin segmentation model to automatically segment portions of an input image that correspond to skin. For example, after training at 802, the automated skin segmentation model can receive an input image, and output a mask corresponding to the input image in which each pixel of the mask corresponds to a portion of the input image (e.g., a pixel of the input image), and each pixel of the mask has a value indicative of whether the corresponding portion of the image is more like skin or not skin.

In some embodiments, one or more sets of labeled training data can be used to train the automated skin segmentation model at 802. The labeled training data can include images in which portions of the images corresponding to skin have been labeled as skin, and portions of the images that correspond to something other than skin (e.g., background, clothing, tattoos, wounds, etc.) have been labeled as non-skin (or not labeled). In some embodiments, the label associated with each training image can be provided in any suitable format. For example, each training image can be associated with a mask in which each pixel of the mask corresponds to a portion of the input image (e.g., a pixel of the input image), and each pixel of the mask has a value indicative of whether the corresponding portion of the image is more like skin or not skin.

Several datasets for evaluating skin segmentation algorithms exist, including HGR (e.g., described in Kawulok, et al., “Self-adaptive algorithm for segmenting skin regions,” EURASIP Journal on Advances in Signal Processing, vol. 2014, no. 1, p. 170, 2014, which is hereby incorporated by reference herein in its entirety), Pratheepan (e.g., as described in Tan, et al. “A fusion approach for efficient human skin detection,” IEEE Transactions on Industrial Informatics, vol. 8, no. 1, pp. 138-147, 2012, which is hereby incorporated by reference herein in its entirety), and ECU (e.g., Casati, et al. “SFA: A human skin image database based on FERET and AR facial images,” in IX workshop de Visao Computational, Rio de Janeiro, 2013, which is hereby incorporated by reference herein in its entirety). HGR includes 1,559 hands images, Pratheepan includes 78 images with the majority of skin pixels from face and hands, and ECU includes 1,118 images with the majority of skin pixels from face and hands. However, abdominal skin pixels were manually segmented from 30 abdominal images, and abdominal skin was shown to have different RGB, HSV, and YCbCr color pixel distributions compared to skin from the HGR and ECU datasets, suggesting that an abdominal dataset encompasses supplementary information on skin features. Due to the gap in existing datasets, adding abdominal skin samples can potentially improve the accuracy of wound, lesion, and/or cancerous region detection, especially if located on an abdomen.

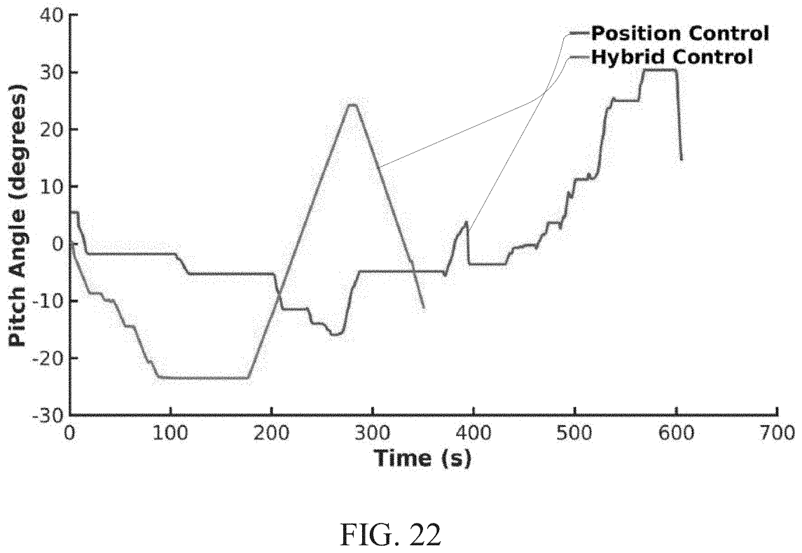

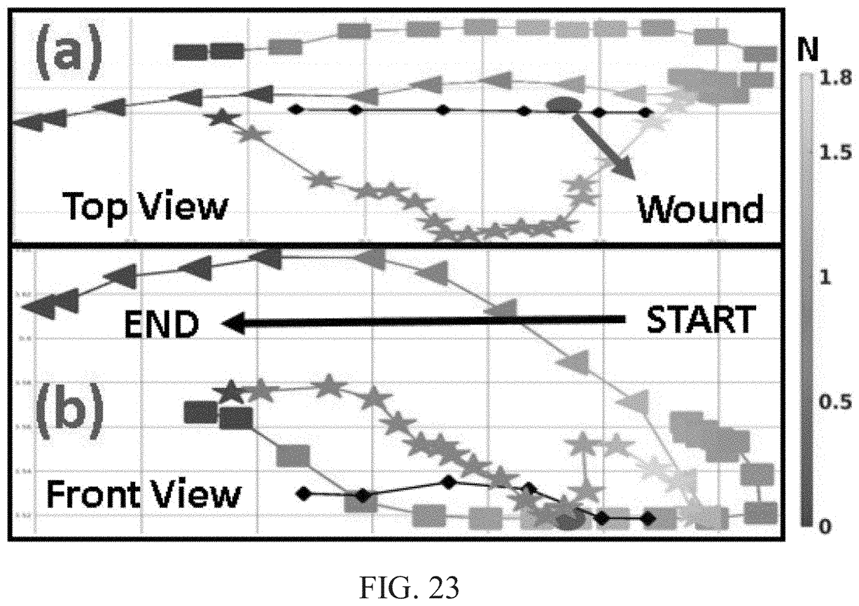

In some embodiments, the process 800 can use images from a dataset of 1,400 abdomen images retrieved from a Google images search, which were subsequently manually segmented. This set of 1,400 images is sometimes referred to herein as the Abdominal skin dataset. The selection and cropping of the images was performed to match the camera's field of observation depicted in FIG. 25B, which is similar to the field of view of the mobile camera 116 described above in connection with, for example, FIGS. 1, 2, 3, and 12B. The images were selected such that the diversity in different ethnic groups is preserved to attempt to prevent indirect racial biases in segmentation techniques. For example, 700 images in the data set represent darker skinned people, which include African, Indian, and Hispanic groups, and 700 images represent lighter skinned people, such as Caucasian and Asian groups. Since the overall complexion of an individual can also provide additional skin features, 400 total images were selected to represent people, with higher body mass indices, split equally between the lighter skinned and darker skinned people categories. Variations between individuals, such as hair and tattoo coverage, in addition to external variations like shadows, were also accounted for in the dataset preparation. Such information can become valuable in hospital and ambulance settings, where lighting and image quality (e.g., degraded quality caused by blurriness due to motion) can vary, hence complicating the skin segmentation task. In addition to the exposed abdominal skin for which the images were selected, other exposed skin regions such as hands, leg parts, and chests were labelled as skin-positive to attempt to prevent the segmentation model from potentially misclassifying skin pixels. In one example, the size of the images was 227×227 pixels. Samples from the data set are shown in, and described below in connection with FIG. 26.

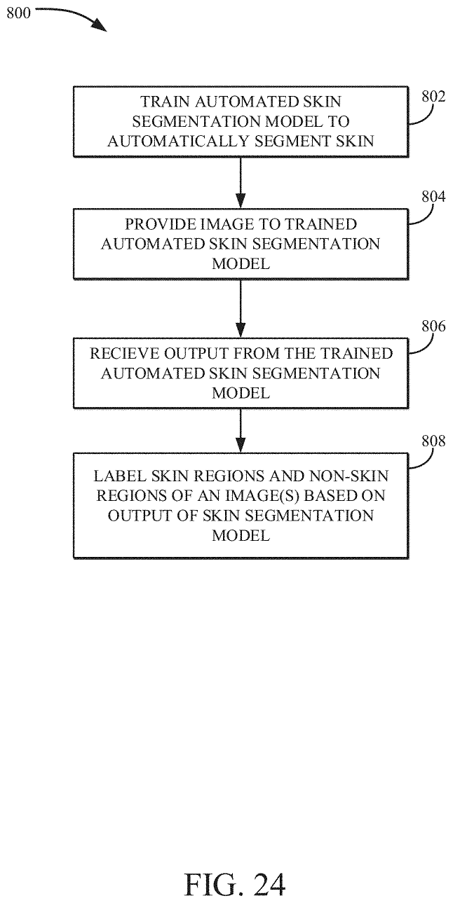

In the images of the data set of 1,400 abdominal images, skin pixels correspond to 66% of the entire pixel data, with a mean of 54.42% per individual image, and a corresponding standard deviation of 15%. As shown FIGS. 27A (non-skin) and 27B (skin), the background (i.e., non-skin) pixels have a relatively consistent distribution across the spectrum, while the skin pixels show a more varied distribution that has some overlay with the non-skin distribution.

In order to form a holistic skin segmentation model, training data from the abdomen as well as other facial and hands datasets can be used, in some embodiments. In some embodiments, at 802, the process 800 can use training images from the Abdominal skin dataset, and images from other skin datasets, such as TDSD, ECU, Schmugge, SFA, HGR, etc. In some embodiments, images included in the training data can be restricted to data sets that are relatively diverse (e.g., in terms of uncontrolled background and lighting conditions). For example, of the five datasets described above, HGR includes the most diverse set of images in terms of uncontrolled background and lighting conditions. Accordingly, in some embodiments, at 802, the process 800 can use training images from the Abdominal skin dataset, and images from the HGR dataset. In some embodiments, the process 800 can divide the training data using an 80-20% training-validation split to generate validation data.

In some embodiments, the process 800 can use any suitable testing data to evaluate the performance of an automated skin segmentation model during training. For example, a test dataset can include images were from various datasets, such as the Abdominal skin dataset, Pratheepan, and ECU. Pratheepan and ECU are established and widely used testing datasets, which were selected to provide results that can be used to compare the performance of an automated skin segmentation model trained in accordance with some embodiments of the disclosed subject matter to existing skin segmentation techniques. In a more particular example, the test dataset can include 200 images from the Abdominal skin dataset that were not included in the training or validation dataset (e.g., 70 from the light lean category, 30 from light obese, 70 from dark lean, and 30 from dark obese). The testing data was selected to attempt to obtain an even evaluation of various segmentation models across different ethnic groups.

In some embodiments, the process 800 can train any suitable type of machine learning model as a skin segmentation model. For example, in some embodiments, the process 800 can train a convolutional network as a skin segmentation model. In general, convolutional networks can be trained to extract relevant features on a highly detailed level, and to compute optimum filters tailored specifically for a particular task. For example, U-Net-based convolutional neural networks are often used for segmenting grayscale images, such as CT and MRI images. An example of a U-Net-based convolutional neural network is described in Ronneberger, et al., “U-net: Convolutional networks for biomedical image segmentation,” International Conference on Medical image computing and computer-assisted intervention, Springer, 2015, pp. 234-241, which is hereby incorporated by reference herein in its entirety.

In some embodiments, the process 800 can train a U-net-based architecture using 128×128 pixel images and three color channels (e.g., R, G, and B channels). For example, such a U-net-based architecture can include a ReLU activation function in all layers, but the output layer can use a sigmoid activation function.

In some embodiments, the process 800 can initialize the weights of the U-net-based model using a sample drawn from a normal distribution centered at zero, with a standard deviation of \sqrt{2/s}, where s is a size of the input tensor. Additionally, the process 800 can fine-tune any suitable parameters for abdominal skin segmentation. For example, the process 800 can use an Adam-based optimizer, which showed superior loss-convergence characteristics as compared to stochastic gradient descent which showed signs of early loss stagnation. As another example, the process 800 can use a learning rate of about 1e-3. As yet another example, the process 800 can use a batch size of 64. In testing, the model loss did not converge with a higher batch size, and the model overfitted to the training data with a smaller batch size. The model converged within 82 epochs using an Adam optimizer, a learning rate of 1e-3, and a batch size of 64.

Additionally or alternatively, in some embodiments, the process 800 can train a Mask-RCNN-based architecture. For example, Mask-RCNN can be described as an extension of Faster-RCNN, whereby a fully convolutional network branch can be added to each region of interest to predict segmentation masks. Mask-RCNN can be well suited to abdominal skin segmentation because of its use of region proposals, which is a different learning algorithm in terms of feature recognition. Additionally, Mask-RCNN makes use of instance segmentation, which can potentially classify the different skin regions as being part of the abdomen, hand, face, etc. In some embodiments, the process 800 can train an architecture similar to the architecture implemented in W. Abdulla, “Mask r-cnn for object detection and instance segmentation on keras and tensorflow,” 2017 (e.g., available via github(dot)com/matterport/mask_RCNN), which is hereby incorporated by reference herein in its entirety, and can use coco weight initialization. In such embodiments, the process 800 can convert the training image datasets into coco format. A smaller resnet50 backbone can be used because it can achieve a faster loss convergence. An anchor ratio can be set to [0.5, 1, 2], and the anchor can be scaled to [8, 16, 32, 64, 128]. Using the coco pre-initialized weights, the network can first be trained for 128 epochs with a learning rate of 1e-4 in order to adapt the weights to the skin dataset(s). The resultant weights can then be used to build a final model, trained using a stochastic gradient descent technique with a learning rate of 1e-3 over 128 epochs. In an example implementation of Mask-RCNN, the selected learning rate converged to an overall loss of 0.52, which is an improvement by 3.6 times over a learning rate of the order of 1e-4.

At 804, the process 800 can provide an image to be segmented to the trained automated skin segmentation model. In some embodiments, the process 800 can format the image to be input into a format that matches the format of the images used to train the automate skin segmentation model. For example, input images can be formatted as 128×128 pixel images to be input to a U-net-based automated skin segmentation model.

At 806, the process 800 can receive an output from the trained automated skin segmentation model indicating which portion or portions of the input image have been identified as corresponding to skin (e.g., abdominal skin), and which portion of portions of the input image have been identified as corresponding to non-skin. In some embodiments, the output can be provided in any suitable format. For example, the output can be provided as a mask that includes an array of pixels in which each pixel of the mask corresponds to a portion of the input image (e.g., a pixel of the input image), and each pixel of the mask has a value indicative of whether the corresponding portion of the image is more like skin or not skin.

At 808, the process 800 can label regions of an image or images as skin and/or non-skin based on the output of the automated skin segmentation model received at 806. In some embodiments, the labeled image can be an original version (or other higher resolution) of the image input to the automated skin segmentation model at 804, prior to formatting for input into the model.

In some embodiments, one or more images that include regions labeled as skin and/or non-skin can be used in any suitable application. For example, in some embodiments, the process 800 can provide labeled images to the computing device 106, the mobile platform 110, and/or the robotic system 114. In such an example, the labeled images can be used to determine portions of a patient that can be scanned using an ultrasound probe (e.g., ultrasound probe 118) and/or to determine portions of the patient that are to be avoided because the regions do not correspond to skin.

FIG. 25A shows an example of a robot arm with an ultrasound probe and a camera positioned to acquire images of a simulated human torso in accordance with some embodiments of the disclosed subject matter, FIG. 25B shows an example of an image of the simulated human torso depicted in FIG. 25A, and FIG. 25C shows a mask corresponding to the image of FIG. 25B generated using techniques described herein for automatically labeling portions of an image corresponding to human skin in accordance with some embodiments of the disclosed subject matter. In some embodiments, abdominal skin detection can facilitate autonomous robotic diagnostics and treatments, such as carrying out autonomous robotic ultrasound routines by localizing the patient with respect to the robot. For example, a robot, such as the robot shown in FIG. 25A, can use a camera to acquire an image of a patient (e.g., an image similar to the image shown in FIG. 25B) using a camera mounted to a robot arm, and mechanisms described herein for automated skin segmentation can be used to automatically label portions of the image corresponding to exposed skin, which the robot can use to determine portions that are appropriate for scanning (e.g., using an ultrasound probe). Isolating skin from wounds and clothes can assist in detecting scannable skin regions, as depicted by the mask shown in FIG. 25C, which can facilitate more autonomous control of ultrasound scans.

FIG. 26 shows examples of images depicting human abdomens that can be used to train an automated skin segmentation model in accordance with some embodiments of the disclosed subject matter. As shown in FIG. 26, the Abdominal skin dataset includes images showing exposed abdominal skin for individuals with diverse characteristics (e.g., diverse skin pigmentation, diverse physiques, diverse tattoo coverage, diverse lighting, diverse body hair coverage, etc.).

FIG. 27A shows an example of an RGB histogram for pixels labeled as being non-skin in a dataset of human abdominal images that can be used to train an automated skin segmentation model in accordance with some embodiments of the disclosed subject matter, and FIG. 27B shows an example of an RGB histogram for pixels labeled as being skin in the dataset of human abdominal images. As shown in FIG. 27A, there is a relatively consistent uniformity across the spectrum in the non-skin portions of the image, with many pixels having R, G, and B values clustered at the extremes. As shown in FIG. 27B, the distribution of R, G, and B values is much smoother in the skin pixels, and more normally distributed. Additionally a comparison of FIGS. 27A and 27B shows overlap between RGB in skin and non-skin pixels, thus encouraging models to account for spatial and contextual relationships to classify the different pixels, rather than relying on only color information.

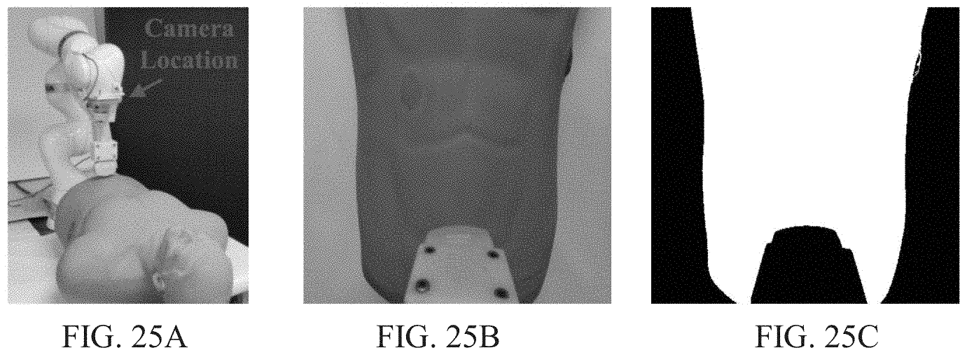

FIG. 28 shows a table of a distribution of images from various datasets used to train and evaluate various automated skin segmentation models implemented in accordance with some embodiments of the disclosed subject matter. The table shown in FIG. 28 provides an overview of the images distributions used for training, validation, and testing of models implemented in accordance with some embodiments of the disclosed subject matter.

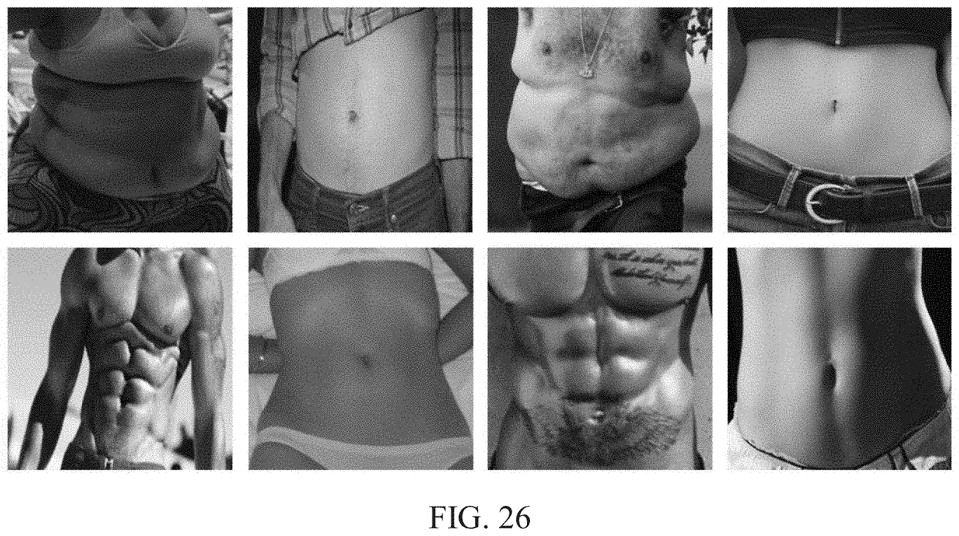

FIG. 29 shows a boxplot depicting the accuracy achieved by various different automated skin segmentation models trained and evaluated using images from the datasets described in connection with FIG. 28. The boxplot shown in FIG. 29 depicts the accuracy of four different models trained using images from the datasets described in connection with FIG. 28, including a U-net-based model (U), a Masked-RCNN-based model (M), a Fully Connected Network model (F), and a naïve Threshold model (T). The networks were trained and tested using four GeForce GTX 1080Ti GPUs (available from Nvidia, Santa Clara, Calif.) with 8 GB of memory each. The total training times for the Fully Connected Network, U-Net, and Mask-RCNN were 6, 5.2 and 13.2 hours, respectively.

The performance of each segmentation model was evaluated based on four image segmentation metrics: accuracy, precision, recall, and F-measure. The formulas for each metric can be represented using the following relationships:

$$Accuracy = \frac{TP + TN}{TP + TN + FP + FN}$$

$$Precision = \frac{TP}{TP + FP}$$

$$Recall = \frac{TP}{TP + FN}$$

$$F \text{-} measure = \frac{2 \times Precision \times Recall}{Precision + Recall}$$

These metrics can provide context for the performance of the networks which are not readily established from accuracy measurements alone, and they can allow comparing the performance of the skin segmentation models described herein to other existing techniques.

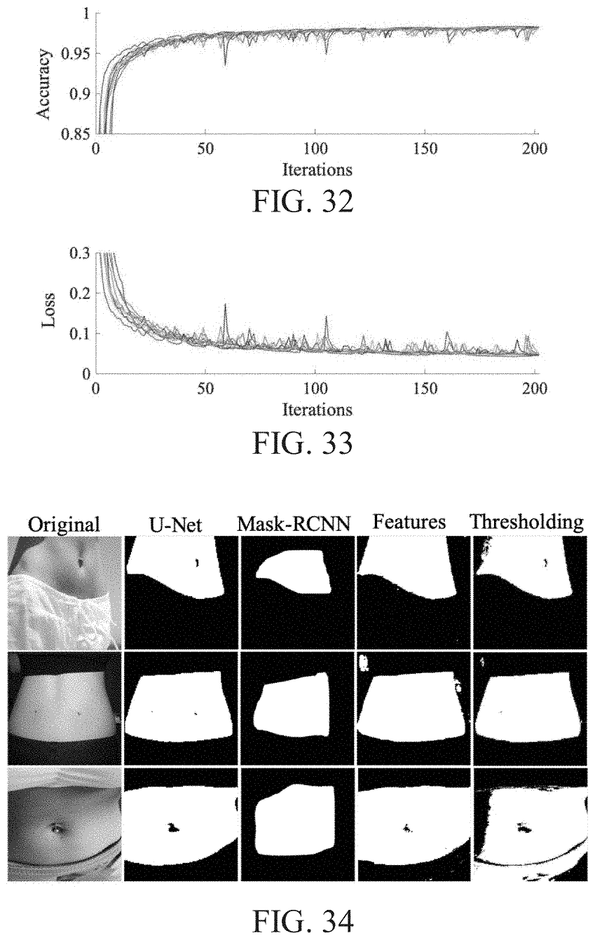

From the boxplot shown in FIG. 29, it can be seen that not only does U-Net exhibit the highest mean accuracy, but it also has the best median results, with 50% of the masks having an accuracy between 93% and 97%. U-Net also resulted in the highest minimum accuracy, with overall least scattered predictions as compared to Mask-RCNN, Fully Connected Network and thresholding. As described below, FIG. 34 shows a visual comparison of segmentation masks generated for the same images by the different techniques evaluated in the boxplot of FIG. 29.

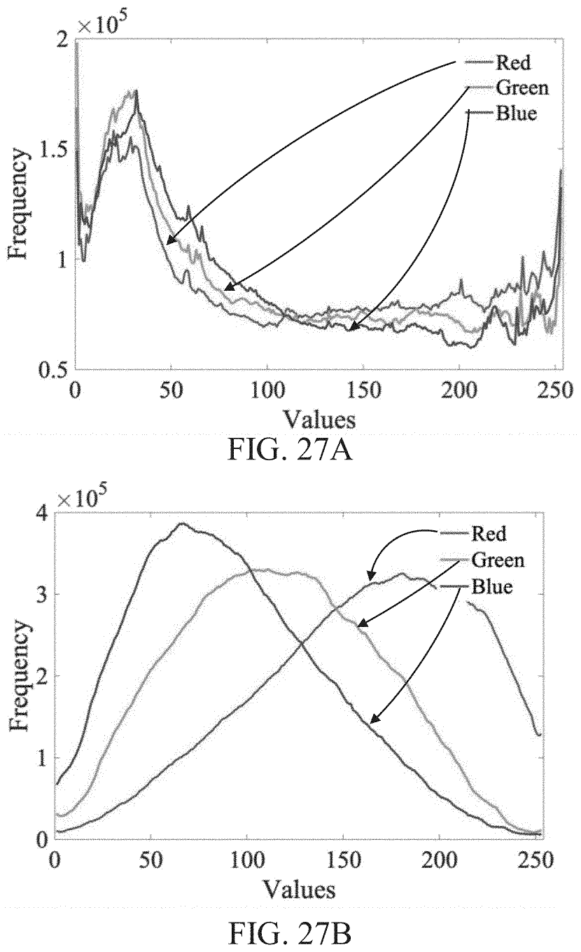

FIG. 30 shows a table of evaluation results for various different automated skin segmentation models trained and evaluated using images from the different combinations of datasets described in connection with FIG. 28, and comparisons with other reported skin segmentation accuracies. Results of thresholding, Fully Connected Network, U-Net and Mask-RCNN over each of the test datasets are shown in FIG. 30. In FIG. 30, the results are based on the accuracy, precision, etc., produced by the model in the first column on a set of images from the dataset identified in the second column.

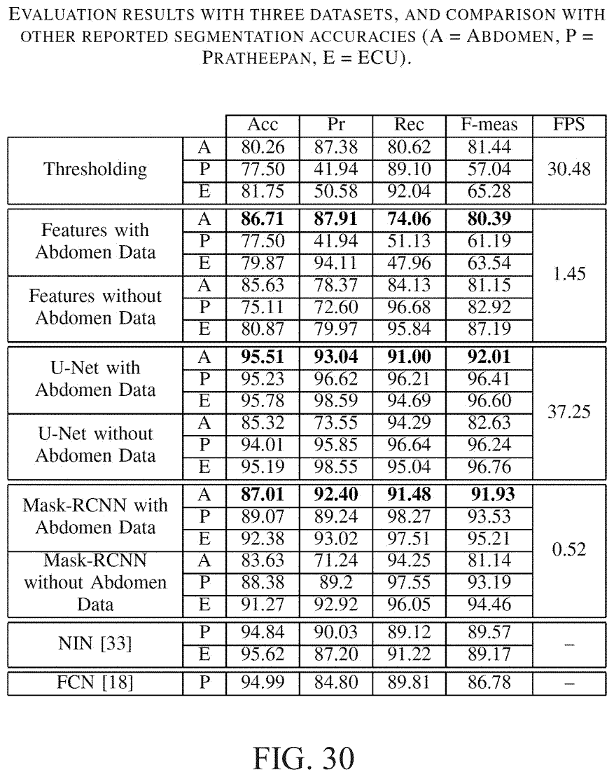

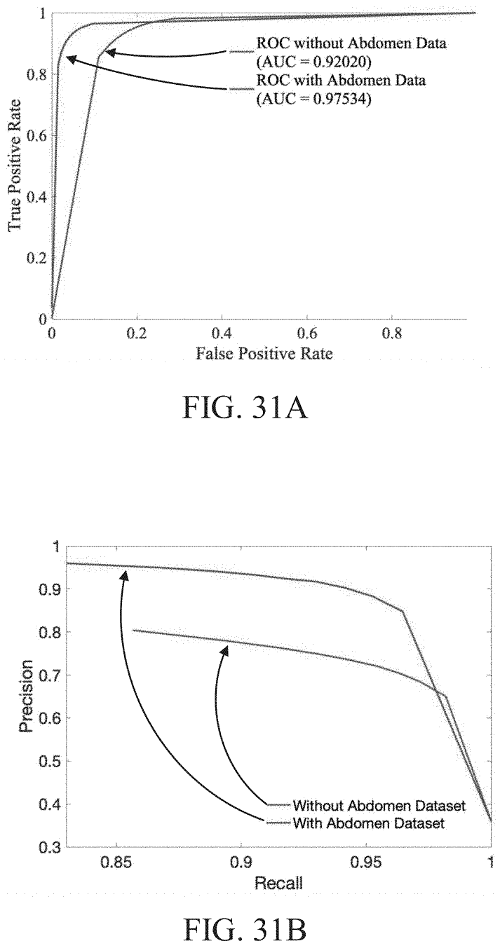

As shown in FIG. 30, U-Net outperformed the other skin segmentation techniques, resulting in the best accuracy of 95.51% for the Abdominal dataset. This accuracy shown in FIG. 30 is the average of 10 different U-Net networks, trained using a 10-fold cross-validation strategy. The cross-validation approach was adopted to ensure the network's robustness, and to rule out the possibility of its high performance being caused by a lucky combination of random numbers. The results of the cross-validation training are shown in FIGS. 32 and 33, where the similarity of the accuracy and loss plots across all 10 folds shows that the Abdominal dataset is balanced and homogeneously diverse. The high precision and recall values with an F-measure of 92.01% also indicate that the trained U-Net network was able to accurately retrieve the majority of the skin pixels.

Mask-RCNN yielded a comparatively lower accuracy of 87.01%, which can be attributed to the poor capabilities of the region proposal algorithm to adapt to the high variability of skin areas in the images. These areas can range from small patches such as fingers, to covering almost the entire image, as is the case with some abdomen images. The variation in the 2D shape of skin regions can also negatively impact the network's performance; the presence of clothing, or any other occlusions caused by non-skin items, results in a different skin shape as perceived by the algorithm.

The fully connected network, which was implicitly designed for finding the optimum decision boundaries for thresholding skin color in RGB, HSV, and YCbCr colorspaces, surpassed the fixed thresholding technique by 6.45%. However, unlike the CNN-based segmentation models, the fully connected network does not account for any spatial relation between the pixels or even textural information, and hence cannot be deemed reliable enough as a stand-alone skin segmentation technique. Due to limitations imposed by the fixed threshold values, neither thresholding nor the fully connected network would be able to produce acceptable segmentation masks with differently colored skin pixels. The maximum accuracy obtained on the Abdomen test set was 86.71% for the Fully Connected Network.

Additionally, to assess the improvement resulting from the addition of the Abdominal dataset into the training set, all three networks were trained both with and without the abdomen images in two separate instances. As shown in FIG. 30, the addition of the Abdominal skin dataset into the training images improved the accuracy performance of U-Net, Mask-RCNN and the Fully Connected Features Network by 10.19%, 3.38%, and 1.08%, respectively, for the Abdominal test dataset. The precision and recall consistently improved for U-Net and Mask-RCNN, as well, demonstrating that the Abdominal skin dataset improved the effectiveness of the networks in correctly retrieving skin pixels.

The thresholding results were generated using a thresholding technique that explicitly delimits boundaries on skin pixel values in pre-determined colorspaces. In general, RGB is not recommended as a stand-alone colorspace, given the high correlation between the R, G, and B values, and their dependency on environmental settings such as lighting. Accordingly, HSV, which has the advantage of being invariance to white light sources, and YCbCr, which separates the luminance (Y) from the chrominance (Cb and Cr), were additionally considered. The decision boundaries were manually optimized for the Abdominal skin dataset, and the final masks used to generate the results shown in FIG. 30 were obtained using the following relationships:

RGB=(R>95)∧(G>40)∧(B>20)∧(R>G)∧(R>B)∧(|R−G|>15)

YCBCR=(Cr>135)∧(Cb>85)∧(Y>80)∧[Cr≤(1.5862Cb+20)]

HSV=(H>0.8)∥(H<0.2)

Final Mask=RGB∥(HSV∧YCbCr)

Note that due to the high dimensional combinatorial aspect of fine-tuning 11 parameters for thresholding (e.g., see the relationships described immediately above in connection with thresholding), the chances of manually determining the optimal values of each parameter are small. The fully connected network (sometimes referred to herein as a fully connected feature network, or features network) results were generated using a fully connected network designed to determine the most suitable decision boundary. The fully connected network included 7 hidden layers with 32, 64, 128, 256, 128, 64, and 32 neurons, respectively. The input layer included 9 neurons, corresponding to the pixel values extracted from the colorspaces as [R, G, B, H, S, V, Y, Cb, Cr], which can be referred to as “features.” The output layer included one neuron for binary pixel classification. The input and hidden layers were followed by a dropout layer each, with a dropout percentage increasing from 10% to 30% towards the end of the network to avoid overfitting. ReLU activation functions were used throughout the network, and the output neuron was activated by a sigmoid. The optimizer used was a momentum stochastic gradient descent (SGD), with a learning rate of 3e-4, a decay of 1e-6, and a momentum of 0.9. The optimizer and corresponding hyperparameters resulted in the best performing features network. The network was trained for 50 epochs.

As shown in FIG. 30, before incorporating the Abdominal dataset into the training images, U-Net resulted in a mean accuracy of 94.01% when tested on the Pratheepan dataset, and 95.19% for 100 images from the ECU dataset. The U-net-based skin segmentation model outperformed all other techniques evaluated. Note that Mask-RCNN performed better on the ECU test dataset than the Pratheepan or Abdominal skin test datasets. This is due to the nature of the images in the three different datasets, whereby in ECU the skin regions have a more consistent shape and size as the dataset is more face oriented. By contrast, in the Pratheepan and Abdominal skin datasets, the skin regions are often cluttered into smaller regions due to clothing occlusions, making it harder for the Mask-RCNN model to propose corresponding skin regions.

The precision and recall for both U-Net and Mask-RCNN over Pratheepan and ECU were higher than the results reported by state-of-the-art networks, such as the image-based Network-in-Network (NIN) configurations described in Kim, et al., “Convolutional neural networks and training strategies for skin detection,” in 2017 IEEE International Conference on Image Processing (ICIP). IEEE, 2017, pp. 3919-3923, and FCN described in Ma, et al., “Human Skin Segmentation Using Fully Convolutional Neural Networks,” in 2018 IEEE 7th Global Conference on Consumer Electronics (GCCE). IEEE, 2018, pp. 168-170, both of which are incorporated herein. This improvement over the existing state of the art networks shows that U-Net and Mask-RCNN networks implemented in accordance with some embodiments of the disclosed subject matter are able to correctly classify almost all of the skin pixels, with a small percentage of non-skin regions incorrectly labeled as skin.

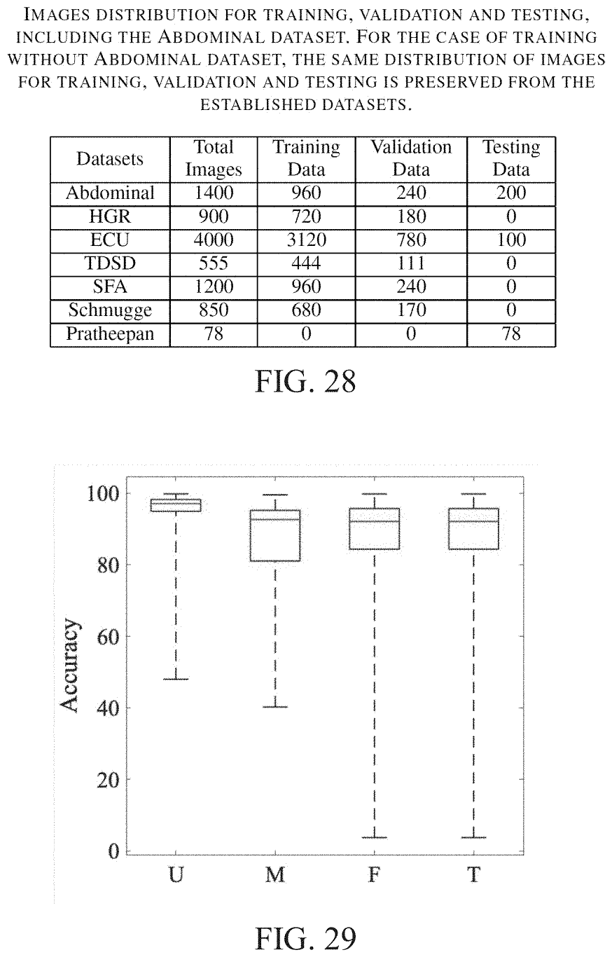

FIG. 31A shows an example of Receiver Operating Characteristic (“ROC”) curves for automated skin segmentation models trained with and without images from the abdominal dataset described in connection with FIG. 28, and FIG. 31B shows an example of Precision-Recall curves for automated skin segmentation models trained with and without images from the abdominal dataset described in connection with FIG. 28. To further analyze the contributions of the Abdominal images, a U-Net-based skin segmentation model was trained with and without the Abdominal skin dataset, and the Receiver Operating Characteristic (“ROC”) and Precision-Recall curves for the models generated with and without are shown in FIGS. 31A and 31B. The two curves show that adding the Abdominal dataset in the training set improved the network's performance, with an increase of 0.045 of the Area Under the Curve (“AUC”) for the ROC curve. This implies that the model trained with Abdominal images is more likely to correctly classify skin pixels and non-skin pixels than its counterpart trained without the Abdominal images.

FIG. 32 shows an example of accuracy achieved during training of various versions of an automated skin segmentation model in a 10-fold cross validations experiment across 200 iterations of training.

FIG. 33 shows an example of a loss value calculated during training of various versions of an automated skin segmentation model in a 10-fold cross validations experiment across 200 iterations of training.

FIG. 34 shows examples of images of human abdomens and masks generated using various techniques for automatically labeling portions of an image corresponding to human skin. As shown in FIG. 34, the U-Net based skin segmentation model produced masks that most faithfully label skin and non-skin in the images.

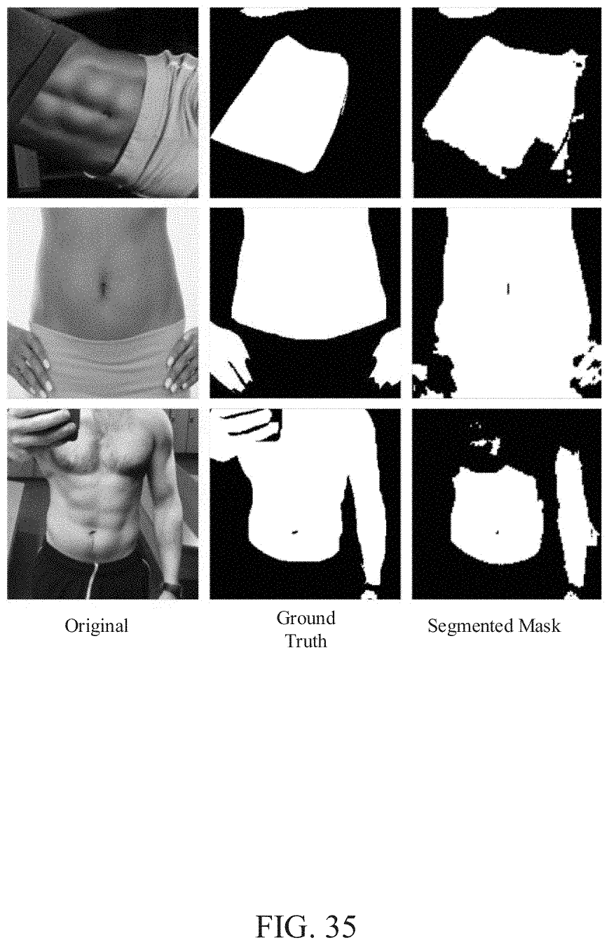

FIG. 35 shows examples of images of human abdomens, manually labeled ground truth masks, and automatically segmented masks generated using an automated skin segmentation model trained in accordance with some embodiments of the disclosed subject matter. Note that although the U-Net-based skin segmentation model was generally the top performer, there were instances of sub-optimal performance. As shown in FIG. 35, the chest region is not segmented uniformly, possibly due to the hair presence, although the U-Net-based model successfully segmented other instances of similar pictures. The other two images depict a common error where orange clothing was mistakenly classified as skin, as the color highly resembles that of skin.

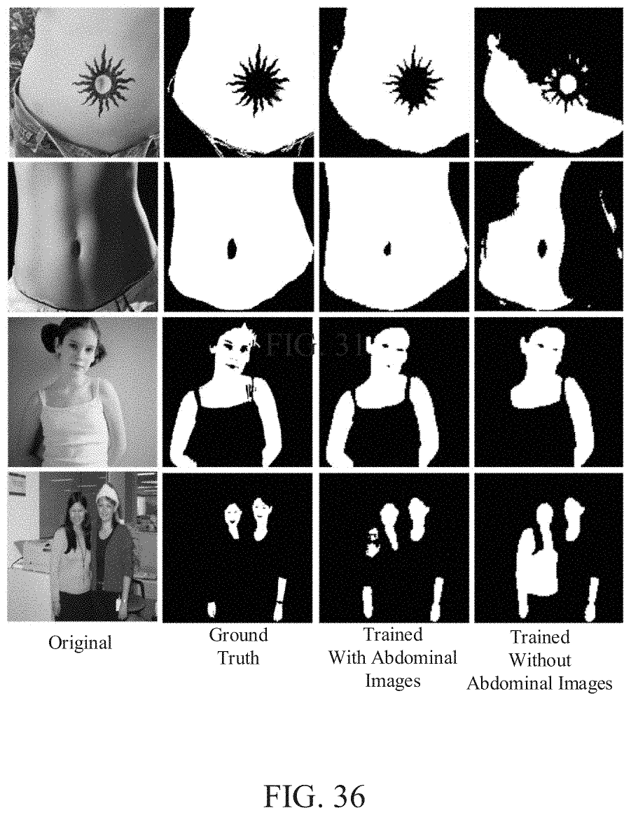

FIG. 36 shows examples of images of human abdomens, manually labeled ground truth masks, and automatically segmented masks generated using an automated skin segmentation model trained in accordance with some embodiments of the disclosed subject matter with and without images from the abdominal dataset described in connection with FIG. 28. FIG. 36 shows the improvement in performance of the U-Net-based skin segmentation model after adding images from the Abdominal skin dataset to the training images for both test images from the Abdominal skin test set and the ECU test set. Not only are the abdomen skin pixels correctly labelled in strong lighting variations, but the hand skin pixels are also correctly identified, and skin colored clothing is correctly classified as non-skin. Since the addition of the Abdominal dataset improved results on both Pratheepan and ECU test images, it can be inferred that the dataset is capable of providing additional information on skin features, rendering skin segmentation algorithms more accurate and holistic.

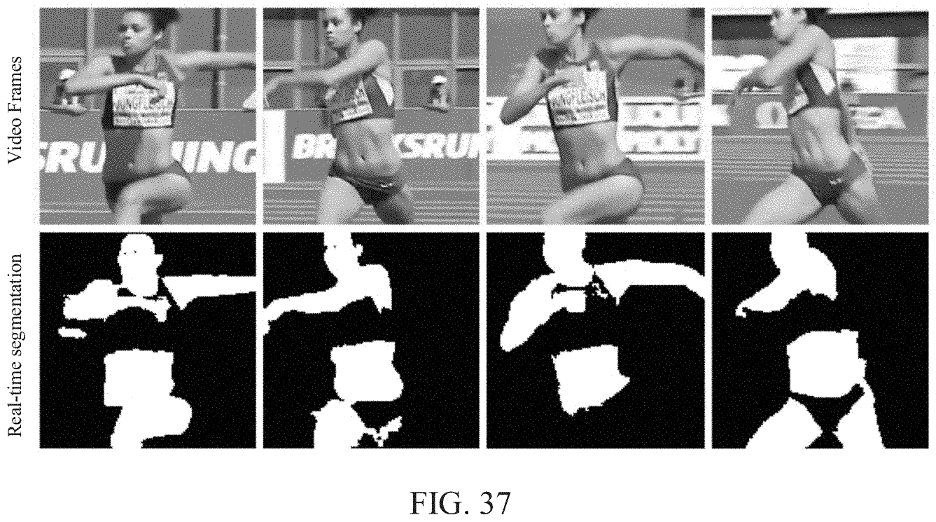

FIG. 37 shows examples of frames of video and corresponding automatically segmented masks generated in real-time using an automated skin segmentation model trained in accordance with some embodiments of the disclosed subject matter. The segmentation speeds for all four techniques described above in connection with FIG. 30 were compared and analyzed by evaluating the frame rate at which images can be evaluated. The networks were tested on CPU memory, meaning that they are easily portable. The frame rate was computed by averaging the time required to segment one frame over the total number of testing images. FIG. 30 shows that U-Net and thresholding segmentation models achieved real-time skin segmentation with speeds of 37.25 frames per second (FPS) and 30.48 FPS, respectively. Mask-RCNN's and the Fully Connected Network's frame rates suffered, however, due to the heavy resnet-50 backbone in Mask-RCNN, and time consuming individual pixel extraction and classification in the Fully Connected Network. A series of frame segmentations by the U-Net-based model are shown in FIG. 37, with a computation time for each frame of 0.0268 seconds.

It should be understood that the above described steps of the processes of FIGS. 5, 6, 8, 11, and 24 can be executed or performed in any order or sequence not limited to the order and sequence shown and described in the figures. Also, some of the above steps of the processes of FIGS. 5, 6, 8, 11, and 24 can be executed or performed substantially simultaneously where appropriate or in parallel to reduce latency and processing times.

In some embodiments, aspects of the present disclosure, including computerized implementations of methods, can be implemented as a system, method, apparatus, or article of manufacture using standard programming or engineering techniques to produce software, which can be firmware, hardware, or any combination thereof to control a processor device, a computer (e.g., a processor device operatively coupled to a memory), or another electronically operated controller to implement aspects detailed herein. Accordingly, for example, embodiments of the invention can be implemented as a set of instructions, tangibly embodied on a non-transitory computer-readable media, such that a processor device can implement the instructions based upon reading the instructions from the computer-readable media. Some embodiments of the invention can include (or utilize) a device such as an automation device, a special purpose or general purpose computer including various computer hardware, software, firmware, and so on, consistent with the discussion below.

The term “article of manufacture” as used herein is intended to encompass a computer program accessible from any computer-readable device, carrier (e.g., non-transitory signals), or media (e.g., non-transitory media). For example, computer-readable media can include but can be not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally, it should be appreciated that a carrier wave can be employed to carry computer-readable electronic data such as those used in transmitting and receiving electronic mail or in accessing a network such as the Internet or a local area network (LAN). Those skilled in the art will recognize many modifications may be made to these configurations without departing from the scope or spirit of the claimed subject matter.

Certain operations of methods according to the invention, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than can be expressly illustrated or described, as appropriate for particular embodiments of the invention. Further, in some embodiments, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” etc. can be intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

As used herein, the term, “controller” and “processor” include any device capable of executing a computer program, or any device that can include logic gates configured to execute the described functionality. For example, this may include a processor, a microcontroller, a field-programmable gate array, a programmable logic controller, etc.

The discussion herein is presented for a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention can be not intended to be limited to embodiments shown, but can be to be accorded the widest scope consistent with the principles and features disclosed herein. The detailed description is to be read with reference to the figures, in which like elements in different figures have like reference numerals. The figures, which can be not necessarily to scale, depict selected embodiments and can be not intended to limit the scope of embodiments of the invention. Skilled artisans will recognize the examples provided herein have many useful alternatives and fall within the scope of embodiments of the invention.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

1. A system for remote trauma assessment, the system comprising:
a robot arm;
an ultrasound probe coupled to the robot arm;
a depth sensor;
a wireless communication system; and
a processor that is programmed to:
cause the depth sensor to acquire depth data indicative of a three dimensional shape of at least a portion of a patient;
generate a 3D model of the patient based on the depth data;
automatically identify, without user input, a plurality of scan positions using the 3D model;
cause the robot arm to move the ultrasound probe to a first scan position of the plurality of scan positions;
receive, from a remote computing device via the wireless communication system, movement information indicative of input to the remote computing device provided via a remotely operated haptic device;
cause the robot arm to move the ultrasound probe from the first scan position to a second position based on the movement information;
cause the ultrasound probe to acquire ultrasound signals at the second position; and
transmit ultrasound data based on the acquired ultrasound signals to the remote computing device.

2. The system of claim 1, further comprising a force sensor coupled to the robot arm, the force sensor configured to sense a force applied to the ultrasound probe and in communication with the processor.

3. The system of claim 2, wherein the processor is further programmed to:
inhibit remote control of the robot arm while the force applied to the ultrasound probe is below a threshold;
determine that the force applied to the ultrasound probe at the first position exceeds the threshold based on a force value received from the force sensor; and
in response to determining that the force applied to the ultrasound probe at the first position exceeds the threshold, accept movement information from the remote computing device.

4. The system of claim 1, further comprising a depth camera comprising the depth sensor, wherein the 3D model is a 3D point cloud, and wherein the processor is further programmed to:
cause the robot arm to move the depth camera to a plurality of positions around a patient;
cause the depth camera to acquire the depth data and corresponding image data at each of the plurality of positions;
generate the 3D point cloud based on the depth data and the image data;
determine a location of the patient's umbilicus using image data depicting the patient;
determine at least one dimension of the patient using the 3D point cloud;
identify the plurality of scan positions based on the location of the patient's umbilicus, the at least one dimension, and a labeled atlas.

5. The system of claim 4, wherein the processor is further programmed to:
provide an image of the patient to a trained machine learning model, wherein the trained machine learning model is a Faster R-CNN that was trained to identify a region of an image corresponding to an umbilicus using labeled training images depicting umbilici;
receive, from the trained machine learning model, an output indicating a location of the patient's umbilicus within the image; and
map the location of the patient's umbilicus within the image to a location on the 3D model.

6. The system of claim 4, wherein the processor is further programmed to:
provide an image of the patient to a trained machine learning model, wherein the trained machine learning model is a Faster R-CNN that was trained to identify a region of an image corresponding to a wound using labeled training images depicting wounds;
receive, from the trained machine learning model, an output indicating a location of a wound within the image;
map the location of the wound within the image to a location on the 3D model; and
cause the robot arm to avoid moving the ultrasound probe within a threshold distance of the wound.

7. The system of claim 6, wherein the processor is further programmed to:
generate an artificial potential field emerging from the location of the wound and having a field strength that decreases with distance from the wound;
determine, based on a position of the ultrasound probe, a force exerted on the ultrasound probe by the artificial potential field;
transmit force information indicative of the force exerted by on the ultrasound probe by the artificial potential field to the remote computing device thereby causing the force exerted on the ultrasound probe by the artificial potential field to be provided as haptic feedback by the haptic device.

8. The system of claim 4, wherein the processor is further programmed to:
provide an image of the patient to a trained machine learning model, wherein the trained machine learning model was trained to identify regions of an image corresponding to objects to be avoided during an ultrasound procedure using labeled training images depicting objects to be avoided during an ultrasound procedure;
receive, from the trained machine learning model, an output indicating one or more locations corresponding to objects to avoid within the image;
map the one or more locations within the image to a location on the 3D model; and
cause the robot arm to avoid moving the ultrasound probe within a threshold distance of the one or more locations.

9. The system of claim 2, wherein the processor is further programmed to:
receive a force value from the force sensor indicative of the force applied to the ultrasound probe; and
transmit force information indicative of the force value to the remote computing device such that the remote computing device displays information indicative of force being applied by the ultrasound probe.

10. The system of claim 1, further comprising a camera, wherein the processor is further programmed to:
receive an image of the patient from the camera;
format the image of the patient for input to an automated skin segmentation model to generate a formatted image;
provide the formatted image to the automated skin segmentation model, wherein the automated skin segmentation model is a U-Net-based model trained using manually segmented a dataset of images that includes a plurality of images that each depict an exposed human abdominal region;
receive, from the automated skin segmentation model, a mask indicating which portions of the image correspond to skin; and
label at least a portion of the 3D model as corresponding to skin based on the mask.

11. A method for remote trauma assessment, comprising:
causing a depth sensor to acquire depth data indicative of a three dimensional shape of at least a portion of a patient;
generating a 3D model of the patient based on the depth data;
automatically identifying, without user input, a plurality of scan positions using the 3D model;
causing the robot arm to move an ultrasound probe mechanically coupled to a distal end of the robot arm to a first scan position of the plurality of scan positions;
receiving, from a remote computing device via a wireless communication system, movement information indicative of input to the remote computing device provided via a remotely operated haptic device;
causing the robot arm to move the ultrasound probe from the first scan position to a second position based on the movement information;
causing the ultrasound probe to acquire ultrasound signals at the second position; and
transmitting ultrasound data based on the acquired ultrasound signals to the remote computing device.

12. The method of claim 11, further comprising determining, using a force sensor coupled to the robot arm, a force applied to the ultrasound probe.

13. The method of claim 12, further comprising:
inhibiting remote control of the robot arm while the force applied to the ultrasound probe is below a threshold;
determining that the force applied to the ultrasound probe at the first position exceeds the threshold based on a force value received from the force sensor; and
in response to determining that the force applied to the ultrasound probe at the first position exceeds the threshold, accepting movement information from the remote computing device.

14. The method of claim 12, wherein the 3D model is a 3D point cloud, the method further comprising:
causing the robot arm to move a depth camera to a plurality of positions around a patient, wherein the depth camera comprises the depth sensor;
causing the depth camera to acquire the depth data and corresponding image data at each of the plurality of positions;
generating the 3D point cloud based on the depth data and the image data;
determining a location of the patient's umbilicus using image data depicting the patient;
determining at least one dimension of the patient using the 3D point cloud;
identifying the plurality of scan positions based on the location of the patient's umbilicus, the at least one dimension, and a labeled atlas.

15. The method of claim 14, further comprising:
providing an image of the patient to a trained machine learning model, wherein the trained machine learning model is a faster R-CNN that was trained to identify a region of an image corresponding to an umbilicus using labeled training images depicting umbilici;
receiving, from the trained machine learning model, an output indicating a location of the patient's umbilicus within the image; and
mapping the location of the patient's umbilicus within the image to a location on the 3D model.

16. The method of claim 12, further comprising:
receiving a force value from the force sensor indicative of the force applied to the ultrasound probe; and
transmitting force information indicative of the force value to the remote computing device such that the remote computing device displays information indicative of force being applied by the ultrasound probe.

17. The method of claim 11, further comprising:
receiving an image of the patient from a camera;
formatting the image of the patient for input to an automated skin segmentation model to generate a formatted image;
providing the formatted image to the automated skin segmentation model, wherein the automated skin segmentation model is a U-Net-based model trained using manually segmented a dataset of images that includes a plurality of images that each depict an exposed human abdominal region;
receiving, from the automated skin segmentation model, a mask indicating which portions of the image correspond to skin; and
labeling at least a portion of the 3D model as corresponding to skin based on the mask.

18. A system for remote trauma assessment, the system comprising:
a haptic device having at least five degrees of freedom;
a user interface;
a display; and
a processor that is programmed to:
cause a graphical user interface comprising a plurality of user interface elements to be presented by the display, the plurality of user interface elements including a switch;
receive, from a remote mobile platform over a communication network, an instruction to enable actuation of the switch;
receive, via the user interface, input indicative of actuation of the switch;
receive, from the haptic device, input indicative of at least one of a position and orientation of the haptic device; and
in response to receiving the input indicative of actuation of the switch, transmit movement information based on the input indicative of at least one of the position and orientation of the haptic device to the mobile platform.

19. The system of claim 18, wherein the haptic device includes an actuatable switch, the actuatable switch having a first position, and a second position, and
wherein the processor is further programmed to:
in response to the actuatable switch being in the first position, cause a robot arm associated with the mobile platform to inhibit translational movements of an ultrasound probe coupled to a distal end of the robot arm along a first axis, a second axis, and a third axis; and
in response to the actuatable switch being in the second position, cause the robot arm to accept translational movement commands that cause the ultrasound probe to translate along at least one of the first axis, the second axis, and the third axis.

20. The system of claim 18, wherein the plurality of user interface elements includes a selectable first user interface element corresponding to a first location, and
wherein the processor is further programmed to:
receive, via the user interface, input indicative of selection of the first user interface element; and
in response to receiving the input indicative of selection of the first user interface element, cause a robot arm associated with the mobile platform to autonomously move an ultrasound probe coupled to a distal end of the robot arm to a first position associated with the first user interface element.

21. The system of claim 20, wherein the processor is further programmed to:
receive force information indicative of a force value generate by a force sensor associated with the mobile device and the robot arm, wherein the force value is indicative of a normal force acting on the ultrasound probe; and
cause the display to present information indicative of the force value based on the information indicative of the force value.

22. The system of claim 21, wherein the processor is further programmed to:
determine that the force value does not exceed a threshold, and
in response to determining that the force value does not exceed a threshold, cause the display to present information indicating that the ultrasound probe is not in contact with a patient to be scanned.

23. The system of claim 20, wherein the processor is further programmed to:
receive, from the mobile platform, image data acquired by a camera associated with the mobile platform, wherein the image data depicts at least a portion of the ultrasound probe;
receive, from the mobile platform, ultrasound data acquired by the ultrasound probe; and
cause the display to simultaneously present an image based on the image data and an ultrasound image based on the ultrasound image.
